(19) United States
(12) Patent Application Publication
(10) Pub. No.: US 2026/0240851 A1
(43) Pub. Date: Aug. 20, 2026

(54) Treatment of Parkinson's Disease in a Patient Using a Glucocerebrosidase Activator

(71) Applicant: BIAL - R&D Investments, S.A., Coronado (S. Romão and S. Mamede) (PT)

(72) Inventors: Nuno Miguel Mendonça da Silva, Coronado (S. Romão and S. Mamede) (PT); Luís Miguel Andrade de Magalhães, Coronado (S. Romão and S. Mamede) (PT); Miguel Mendonça da Fonseca, Coronado (S. Romão and S. Mamede) (PT); Guillermo Castilla Fernandez, Coronado (S. Romão and S. Mamede) (PT); Dana Hilt, Coronado (S. Romão and S. Mamede) (PT); David Keith Simon, Coronado (S. Romão and S. Mamede) (PT); Joerg Holenz, Coronado (S. Romão and S. Mamede) (PT); Amir Atabak Ronaghinia, Coronado (S. Romão and S. Mamede) (PT)

(21) Appl. No.: 19/160,807

(22) PCT Filed: Mar. 11, 2024
(86) PCT No.: PCT/PT2024/050012

§ 371 Date: Aug. 28, 2025

Related U.S. Application Data
(60) Provisional application No. 63/451,416, filed on Mar. 10, 2023.
(60) Provisional application No. 63/452,382, filed on Mar. 15, 2023.
(60) Provisional application No. 63/455,454, filed on Mar. 29, 2023.
(60) Provisional application No. 63/457,298, filed on Apr. 5, 2023.
(60) Provisional application No. 63/613,291, filed on Dec. 21, 2023.

(51) Int. Cl. A61K 31/519; A61K 31/198; A61K 45/06; A61P 25/16

(52) U.S. Cl. CPC A61K 31/519; A61K 31/198; A61K 45/06; A61P 25/16

(57) ABSTRACT

Methods for preventing, limiting or delaying clinical motor progression in a subject with Parkinson's disease with low GCase activity, such as a PD patient with a pathogenic variant in the glucocerebrosidase 1 (GBA1) gene (GBA-PD) is provided, said methods comprising administering a therapeutically effective amount of 5,7-dimethyl-N-((1R,4R)-4-(pentyloxy)cyclohexyl)pyrazolol[1,5-a]pyrimidine-3-carboxamide (Compound A), or a pharmaceutically acceptable salt thereof, to said subject.

### FIELD OF THE INVENTION

The present invention relates to 5,7-dimethyl-N-((1R,4R)-4-(pentyloxy)cyclohexyl)pyrazolol[1,5-a]pyrimidine-3-carboxamide for use in the treatment or prevention of Parkinson's Disease.

### BACKGROUND TO THE INVENTION

Parkinson disease (PD) is a multicentric neurodegenerative disease characterised pathologically by the loss of dopaminergic neurons in the substantia nigra pars compacta and other brain stem nuclei, as well as by the presence of alpha-synuclein (SNCA) aggregates in Lewy bodies and neurites.

GCase which is encoded by the GBA1 gene is the enzyme responsible for the hydrolysis of glucosylceramide (GluCer) to glucose and ceramide in the lysosomes. GCase activity dysfunction has been implicated in Parkinson's Disease. GCase mutations are known to be a major risk factor for the onset of PD. Loss of GCase function contributes to the pathogenesis of PD, even independently of genetic mutation.

Even carrying one mutated allele of GBA1 significantly increases the lifetime risk of developing PD. Approximately 10% to 15% of patients with clinically diagnosed PD have a GBA1 pathogenic variant (GBA-PD). The degree of increased risk of being diagnosed with PD conferred by a heterozygous pathogenic variant depends on the magnitude of reduction of GCase activity and can range from 2.2- to 19.2-fold. This increased risk for PD has been demonstrated for over 100 GBA1 pathogenic variants in large cohorts in the United States (Grabowski, 2008), Israel (Guimarães, 2012), Sweden (Jesús, 2016), Spain (Liu, 2016), the United Kingdom (Mata, 2016), Greece (Moraitou, 2011), China (Neudorfer, 1996), and South America (Neumann, 2009) and in various meta-analyses (Gan-Or, 2015). In Israel, approximately 20% to 30% of patients with PD and an Ashkenazi Jewish background are carriers of GBA1 pathogenic variants. Thus, pathogenic variants in GBA1 are the most common genetic risk factor for PD in a variety of clinical conditions.

As a group, patients with GBA-PD generally have a clinically distinct course when compared to patients with idiopathic PD (i.e., without a GBA1 pathogenic variant). On average, patients with GBA-PD present with an earlier age of onset (Grabowski, 2008; Gan-Or, 2015). They exhibit a relative prominence of postural instability gait disturbance over tremor, and their disease is characterized by frequent falls. Typically, patients with GBA-PD progress more rapidly than those with idiopathic PD (iPD) (Gan-Or, 2008; Ran, 2016), and on average their survival from time of diagnosis is shorter (Rosenbloom, 2011). Patients with GBA-PD also exhibit more frequent cognitive dysfunction than patients with iPD, with more rapid progression to dementia (Rosenbloom, 2013; Pal, 2016; Ran, 2016). GBA-PD is further characterized by greater prevalence of depression, anxiety, hallucinations, and rapid eye movement sleep behavior disorder (Liu, 2016; Mata, 2016). It has recently been reported that, among the many known PD genetic risk factors, only GBA1 increases the rate of disease progression (Tan, 2021).

Despite this, low/reduced GCase activity has also been observed in certain PD patients not carrying a GBA1 pathogenic variant (e.g. wild-type GBA1 PD patients) (Moloney, 2021).

5,7-dimethyl-N-((1R,4R)-4-(pentyloxy)cyclohexyl)pyrazolol[1,5-a]pyrimidine-3-carboxamide (herein also referred to as “Compound A”) is an allosteric activator of the enzyme beta-glucocerebrosidase (GCase). Compound A is therefore a potential treatment for Parkinson's Disease (PD) for patients with a mutation in the GBA1 gene (GBA-PD) and more general, for patients that have low/reduced GCase activity, that could benefit from a treatment option which increases GCase activity.

Compound A has been shown to be safe for administration to healthy humans (J. M den Heijer, 2021). It was generally well tolerated, no treatment-related serious adverse events or deaths occurred, and no subject withdrew from the study due to adverse events so far in the clinical development plan.

Currently available pharmacotherapy for PD is primarily directed at the dopaminergic system and primarily alleviates motor symptomatology, without addressing non-motor symptoms or otherwise influencing/modifying disease progression. Despite the promise shown in various preclinical studies of PD disease modifiers, there are no successful clinical disease modifiers. Similarly, whilst there have been attempts to modify disease progression in patients with GBA-PD, there have been no successful clinical candidates. For example, MOVES-PD was a global Phase 2 study to assess the efficacy and safety of the drug candidate venglustat in Parkinson's patients who have a GBA-1 genetic mutation. Despite promising preclinical studies, when the final data was collected, the results of the study indicated that the drug had no impact on slowing the progression of the condition (Peterschmitt et al., 2021a,b).

This leaves a large unmet need for disease-modifying Parkinson's therapies.

Additionally, there is also need for methods for administering Compound A, to a patient in need thereof, wherein the patient also is being administered other concomitant drugs. The present disclosure fulfills these and other needs, as evident in reference to the following disclosure.

### SUMMARY OF THE INVENTION

In a first aspect of the invention, there is described Compound A, or a pharmaceutically acceptable salt thereof, for use in preventing or limiting clinical motor progression in a subject with Parkinson's disease who has a pathogenic variant in the glucocerebrosidase 1 (GBA1) gene (GBA-PD). According to an embodiment clinical motor progression is assessed using the Movement Disorder Society-Unified Parkinson's Disease Rating Scale (MDS-UPDRS), the Clinical Global Impression-Change (CGI-C) scale, and/or the Patient Global Impression-Change (PGI-C) scale. In certain embodiments, clinical motor progression is also assessed using the modified Hoehn and Yahr Score, the 39-item Parkinson's Disease questionnaire (PDQ-39) score, and/or the EuroQol 5 Dimension 5 Level (EQ-5D-5L) score.

In a further aspect of the invention, there is described a method for preventing or limiting clinical motor progression in a subject with Parkinson's disease who has a pathogenic variant in the glucocerebrosidase 1 (GBA1) gene (GBA-PD), said method comprising administering a therapeutically effective amount of Compound A, or a pharmaceutically acceptable salt thereof, to said subject. According to an embodiment clinical motor progression is assessed using the Movement Disorder Society-Unified Parkinson's Disease Rating Scale (MDS-UPDRS), the Clinical Global Impression-Change (CGI-C) scale, and/or the Patient Global Impression-Change (PGI-C) scale. In certain embodiments, clinical motor progression is also assessed using the modified Hoehn and Yahr Score, the 39-item Parkinson's Disease questionnaire (PDQ-39) score, and/or the EuroQol 5 Dimension 5 Level (EQ-5D-5L) score.

There has been a lot of research into preventing or limiting clinical motor progression in subjects with Parkinson's disease by activating GCase. However, despite successful ex vivo studies demonstrating effective activation of GCase, there has been no demonstration of the prevention or limitation of clinical motor progression in a subject with Parkinson's Disease.

It is also noted that despite the success of disease-modifying interventions in preclinical models, this has not translated into effective clinical success. No commercially available pharmaceutical agents have the ability to stop, prevent or mitigate clinical motor progression in PD. Without wishing to be bound by theory, it is postulated that one reason for the failure to translate preclinical success to clinical success is the multiple different pathophysiologies of PD and the existence of heterogeneity among individual patients. It is therefore very difficult to predict clinical success. Recently failed trials include various NET-PD trials (Tilley and Galpern, 2007), the ADAGIO trial (Rascol et al., 2016), the more recent α-Synuclein immunotherapy trials (ClinicalTrials.gov, 2021) and the Venglustat trial (Peterschmitt et al., 2021a,b).

The present invention derives from the finding that Compound A can be effective at preventing, limiting or delaying motor progression in certain PD subjects, particularly PD patients with decreased, reduced, or low GCase activity, for example, GBA-PD patients. That is, Compound A is capable of treating the underlying pathology of PD for these patients rather than simply treating the symptoms of PD.

The therapy involves subjects with Parkinson's disease who have a pathogenic variant in the glucocerebrosidase 1 (GBA1) gene (GBA-PD). A number of pathological/pathogenic variations in the GBA1 gene are known which affect the activity of the GCase enzyme. Pathological/pathogenic variations in the GBA1 gene include but are not limited to N370S, D409H, H255Q, D140H, G202R, L324P, I260T, L444P, A190T, and R120W. Major/Severe mutations include but are not limited to heterozygous for D409H, H255Q, D140H, G202R, L324P, I260T, L444P, A190T, R120W. Minor/mild mutations include but are not limited to heterozygous for T369M or E326K. Further information on subjects being heterozygous for a pathogenic variant in the GBA1 gene is provided below.

Further pathological/pathogenic variations in the GBA1 gene include but are not limited to those included in the table in Appendix 1. This includes but is not limited to a GBA1 gene containing one of the following nucleotide variations: NM_001005741.3:c.1093G>A, NM_001005741.3:c.1223C>T, NM_001005741.3:c.1226A>G, NM_001005741.3:c.1448T>C, NM_001005741.3:c.1604G>A, NM_001005741.3:c.1342G>C, NM_001005741.3:c.680A>G, NM_001005741.3:c.1504C>T, NM_001005741.3:c.84dup, NM_001005741.3:c.754T>A, NM_001005741.3:c.1297G>T, NM_001005741.3:c.721G>A, NM_001005741.3:c.259C>T, NM_001005741.3:c.928A>G, NM_001005741.3:c.764T>A, NM_001005741.3:c.1246G>A, NM_001005741.3:c.946C>T, NM_001005741.3:c.26_27del, NM_001005741.3:c.604C>T, NM_001005741.3:c.1090G>A, NM_001005741.3:c.1296G>A, NM_001005741.3:c.1192C>T, NM_001005741.3:c.914del, NM_001005741.3:c.256C>T, NM_001005741.3:c.586A>C, NM_001005741.3:c.1312G>A, NM_001005741.3:c.203dup, NM_001005741.3:c.475C>T, NM_001005741.3:c.476G>A, NM_001005741.3:c.887G>A, NM_001005741.3:c.762-1G>C, NM_001005741.3:c.115+1G>A, NM 001005741.3: c.1505+1G>T, NM_001005741.3:c.123_217del, NM_001005741.2:c.1265_1319del, NM_001005741.3:c.715C>T, NM_001005741.3:c.1085C>T, NM_001005741.3:c.413del, NM_001005741.3:c.882T>G, NM_001005741.3:c.1193G>A and NM_001005741.3:c.1_2344del. In some embodiments, this includes but is not limited to a GBA1 gene containing one of the following nucleotide variations: NM_001005741.3:c.1093G>A, NM_001005741.3:c.1223C>T, NM_001005741.3:c.1226A>G, NM_001005741.3:c.1448T>C, NM_001005741.3:c.1604G>A, NM_001005741.3:c.1342G>C, NM_001005741.3:c.680A>G, NM_001005741.3:c.1504C>T, NM_001005741.3:c.84dup, NM_001005741.3:c.754T>A, NM_001005741.3:c.1297G>T, NM_001005741.3:c.721G>A, NM_001005741.3:c.259C>T, NM_001005741.3:c.928A>G, NM_001005741.3:c.764T>A, NM_001005741.3:c.1246G>A, NM_001005741.3:c.946C>T, NM_001005741.3:c.26_27del, NM_001005741.3:c.604C>T, NM_001005741.3:c.1090G>A, NM_001005741.3:c.1296G>A, NM_001005741.3:c.1192C>T, NM_001005741.3:c.914del, NM_001005741.3:c.256C>T, NM_001005741.3:c.586A>C, NM_001005741.3:c.1312G>A, NM_001005741.3:c.203dup, NM_001005741.3:c.475C>T, NM_001005741.3:c.476G>A, NM_001005741.3:c.887G>A, NM_001005741.3:c.762-1G>C, NM_001005741.3:c.115+1G>A, and NM_001005741.3:c.1505+1G>T. Major/Severe mutations include but are not limited to NM_001005741.3:c.1448T>C, NM_001005741.3:c.1342G>C, NM_001005741.3:c.680A>G, NM_001005741.3:c.1504C>T, NM_001005741.3:c.84dup, NM_001005741.3:c.754T>A, NM_001005741.3:c.1297G>T, NM_001005741.3:c.721G>A, NM_001005741.3:c.1246G>A, NM_001005741.3:c.26_27del, NM_001005741.3:c.604C>T, NM_001005741.3:c.1090G>A, NM_001005741.3:c.1296G>A, NM_001005741.3:c.1192C>T, NM_001005741.3:c.914del, NM_001005741.3:c.256C>T, NM_001005741.3:c.586A>C, NM_001005741.3:c.1312G>A, NM_001005741.3:c.203dup, NM_001005741.3:c.475C>T, NM_001005741.3:c.476G>A, NM_001005741.3:c.887G>A, NM_001005741.3:c.762-1G>C, NM_001005741.3:c.115+1G>A, NM_001005741.3:c.1505+1G>T, NM_001005741.3:c.123_217del, NM_001005741.2:c.1265_1319del, NM_001005741.3:c.715C>T, NM_001005741.3:c.1085C>T, NM_001005741.3:c.413del, NM_001005741.3:c.882T>G and NM_001005741.3:c.1_2344del. In some embodiments, Major/Severe mutations include but are not limited to NM_001005741.3:c.1448T>C, NM_001005741.3:c.1342G>C, NM_001005741.3:c.680A>G, NM_001005741.3:c.1504C>T, NM_001005741.3:c.84dup, NM_001005741.3:c.754T>A, NM_001005741.3:c.1297G>T, NM_001005741.3:c.721G>A, NM_001005741.3:c.1246G>A, NM_001005741.3:c.26_27del, NM_001005741.3:c.604C>T, NM_001005741.3:c.1090G>A, NM_001005741.3:c.1296G>A, NM_001005741.3:c.1192C>T, NM_001005741.3:c.914del, NM_001005741.3:c.256C>T, NM_001005741.3:c.586A>C, NM_001005741.3:c.1312G>A, NM_001005741.3:c.203dup, NM_001005741.3:c.475C>T, NM_001005741.3:c.476G>A, NM_001005741.3:c.887G>A, NM_001005741.3:c.762-1G>C, NM_001005741.3:c.115+1G>A, and NM_001005741.3:c.1505+1G>T. Minor/mild mutations include but are not limited to NM_001005741.3:c.1093G>A, NM_001005741.3:c.1223C>T, NM_001005741.3:c.1226A>G, NM_001005741.3:c.1604G>A, NM_001005741.3:c.259C>T, NM_001005741.3:c.928A>G, NM_001005741.3:c.764T>A, NM_001005741.3:c.946C>T and NM_001005741.3:c.1193G>A. In some embodiments, minor/mild mutations include but are not limited to NM_001005741.3:c.1093G>A, NM_001005741.3:c.1223C>T, NM_001005741.3:c.1226A>G, NM_001005741.3:c.1604G>A, NM_001005741.3:c.259C>T, NM_001005741.3:c.928A>G, NM_001005741.3:c.764T>A, and NM_001005741.3:c.946C>T.

In terms of the GCase amino acid sequence, a pathological/pathogenic variation in the GCase amino acid sequence includes but is not limited to those included in the table in Appendix 1. This includes but is not limited to a GBA1 gene encoding a GCase enzyme containing one of the following amino acid variations: p.Glu365Lys, p.Thr408Met, p.Asn409Ser, p.Leu483Pro, p.Arg535His, p.Asp448His, p.Asn227Ser, p.Arg502Cys, p.Leu29Alafs*18, p.Phe252Ile, p.Val433Leu, p.Gly241Arg, p.Arg87Trp, p.Ser310Gly, p.Phe255Tyr, p.Gly416Ser, p.Arg316Cys, p.Glu9Glyfs*8, p.Arg202*, p.Gly364Arg, p.Trp432*, p.Arg398*, p.Pro305Leufs*31, p.Arg86*, p.Lys196Gln, p.Asp438Asn, p.Thr69Aspfs*12, p.Arg159Trp, p.Arg159Gln, p.Arg296Gln, p.Pro42Trpfs*7, p.Pro42TrpfsTer7, p.Leu422ProfsTer4, p.Leu422Profs*4, p.Gln239*, p.Gln239Ter, p.Thr362Ile, p.Pro138Leufs*62, p.Pro138LeufsTer62, p.His294Gln, p.Arg398Gln and p.Met1_*537del. In some embodiments, this includes but is not limited to a GBA1 gene encoding a GCase enzyme containing one of the following amino acid variations: p.Glu365Lys, p.Thr408Met, p.Asn409Ser, p.Leu483Pro, p.Arg535His, p.Asp448His, p.Asn227Ser, p.Arg502Cys, p.Leu29Alafs*18, p.Phe252Ile, p.Val433Leu, p.Gly241Arg, p.Arg87Trp, p.Ser310Gly, p.Phe255Tyr, p.Gly416Ser, p.Arg316Cys, p.Glu9Glyfs*8, p.Arg202*, p.Gly364Arg, p.Trp432*, p.Arg398*, p.Pro305Leufs*31, p.Arg86*, p.Lys196Gln, p.Asp438Asn, p.Thr69Aspfs*12, p.Arg159Trp, p.Arg159Gln, and p.Arg296Gln. Major/Severe mutations include but are not limited to p.Leu483Pro, p.Asp448His, p.Asn227Ser, p.Arg502Cys, p.Leu29Alafs*18, p.Phe252Ile, p.Val433Leu, p.Gly241Arg, p.Gly416Ser, p.Glu9Glyfs*8, p.Arg202*, p.Gly364Arg, p.Trp432*, p.Arg398*, p.Pro305Leufs*31, p.Arg86*, p.Lys196Gln, p.Asp438Asn, p.Thr69Aspfs*12, p.Arg159Trp, p.Arg159Gln, p.Arg296Gln, p.Pro42Trpfs*7, p.Pro42TrpfsTer7, p.Leu422ProfsTer4, p.Leu422Profs*4, p.Gln239*, p.Gln239Ter, p.Thr362Ile, p.Pro138Leufs*62, p.Pro138LeufsTer62, p.His294Gln and p.Met1_*537del. In some embodiments, Major/Severe mutations include but are not limited to p.Leu483Pro, p.Asp448His, p.Asn227Ser, p.Arg502Cys, p.Leu29Alafs*18, p.Phe252Ile, p.Val433Leu, p.Gly241Arg, p.Gly416Ser, p.Glu9Glyfs*8, p.Arg202*, p.Gly364Arg, p.Trp432*, p.Arg398*, p.Pro305Leufs*31, p.Arg86*, p.Lys196Gln, p.Asp438Asn, p.Thr69Aspfs*12, p.Arg159Trp, p.Arg159Gln, and p.Arg296Gln. Minor/mild mutations include but are not limited to p.Glu365Lys, p.Thr408Met, p.Asn409Ser, p.Arg535His, p.Arg87Trp, p.Ser310Gly, p.Phe255Tyr, p.Arg316Cys and p.Arg398Gln. In some embodiments, minor/mild mutations include but are not limited to p.Glu365Lys, p.Thr408Met, p.Asn409Ser, p.Arg535His, p.Arg87Trp, p.Ser310Gly, p.Phe255Tyr, and p.Arg316Cys.

In the aspects above and additional aspects below, Compound A is used or administered to a subject with Parkinson's disease who has a pathogenic variant in the glucocerebrosidase 1 (GBA1) gene (GBA-PD). A subject will have two alleles of the GBA1 gene, both of which will have an impact on the total GCase activity within the cells of the subject. Therefore, in some embodiments, the subject is heterozygous for the GBA1 gene having one allele which contains a pathogenic variant and one allele which is non-pathogenic (such as the wild-type allele for the GBA1 gene). In other embodiments, the subject is heterozygous for the GBA1 gene having one deleted allele (i.e. complete gene deletion) and one allele which is non-pathogenic (such as the wild-type allele for the GBA1 gene). The term ‘heterozygous’ in this disclosure, where the second allele is not defined, should be interpreted as meaning that the second allele is non-pathogenic (such as the wild-type allele). This is in contrast to the term ‘compound heterozygous’ which is used to mean both alleles of the GBA1 gene contain at least one pathogenic variant, said pathogenic variants not being the same between the two alleles. The term ‘homozygous’ means both alleles of the GBA1 gene contain the same pathogenic variant(s).

In some embodiments, the subject is heterozygous for the GBA1 gene having one allele which contains one of the following nucleotide variations: NM_001005741.3:c.1093G>A, NM_001005741.3:c.1223C>T, NM_001005741.3:c.1226A>G, NM_001005741.3:c.1448T>C, NM_001005741.3:c.1604G>A, NM_001005741.3:c.1342G>C, NM_001005741.3:c.680A>G, NM_001005741.3:c.1504C>T, NM_001005741.3:c.84dup, NM_001005741.3:c.754T>A, NM_001005741.3:c.1297G>T, NM_001005741.3:c.721G>A, NM_001005741.3:c.259C>T, NM_001005741.3:c.928A>G, NM_001005741.3:c.764T>A, NM_001005741.3:c.1246G>A, NM_001005741.3:c.946C>T, NM_001005741.3:c.26_27del, NM_001005741.3:c.604C>T, NM_001005741.3:c.1090G>A, NM_001005741.3:c.1296G>A, NM_001005741.3:c.1192C>T, NM_001005741.3:c.914del, NM_001005741.3:c.256C>T, NM_001005741.3:c.586A>C, NM_001005741.3:c.1312G>A, NM_001005741.3:c.203dup, NM_001005741.3:c.475C>T, NM_001005741.3:c.476G>A, NM_001005741.3:c.887G>A, NM_001005741.3:c.762-1G>C, NM_001005741.3:c.115+1G>A, NM_001005741.3:c.1505+1G>T, NM_001005741.3:c.123_217del, NM_001005741.2:c.1265_1319del, NM_001005741.3:c.715C>T, NM_001005741.3:c.1085C>T, NM_001005741.3:c.413del, NM_001005741.3:c.882T>G, NM_001005741.3:c.1193G>A and NM_001005741.3:c.1_2344del (with the other allele being non-pathogenic). In some embodiments, the subject is heterozygous for the GBA1 gene having one allele which contains one of the following nucleotide variations: NM_001005741.3:c.1093G>A, NM_001005741.3:c.1223C>T, NM_001005741.3:c.1226A>G, NM_001005741.3:c.1448T>C, NM_001005741.3:c.1604G>A, NM_001005741.3:c.1342G>C, NM_001005741.3:c.680A>G, NM_001005741.3:c.1504C>T, NM_001005741.3:c.84dup, NM_001005741.3:c.754T>A, NM_001005741.3:c.1297G>T, NM_001005741.3:c.721G>A, NM_001005741.3:c.259C>T, NM_001005741.3:c.928A>G, NM_001005741.3:c.764T>A, NM_001005741.3:c.1246G>A, NM_001005741.3:c.946C>T, NM_001005741.3:c.26_27del, NM_001005741.3:c.604C>T, NM_001005741.3:c.1090G>A, NM_001005741.3:c.1296G>A, NM_001005741.3:c.1192C>T, NM_001005741.3:c.914del, NM_001005741.3:c.256C>T, NM_001005741.3:c.586A>C, NM_001005741.3:c.1312G>A, NM_001005741.3:c.203dup, NM_001005741.3:c.475C>T, NM_001005741.3:c.476G>A, NM_001005741.3:c.887G>A, NM_001005741.3:c.762-1G>C, NM_001005741.3:c.115+1G>A, and NM_001005741.3:c.1505+1G>T (with the other allele being non-pathogenic). In addition, the subject may be homozygous for the GBA1 gene having either the pathogenic variant NM_001005741.3:c.1093G>A or the pathogenic variant NM_001005741.3:c.1223C>T.

In terms of the GCase amino acid sequence, the subject may be heterozygous for the GBA1 gene having one allele which encodes a GCase enzyme containing one of the following amino acid variations: p.Glu365Lys, p.Thr408Met, p.Asn409Ser, p.Leu483Pro, p.Arg535His, p.Asp448His, p.Asn227Ser, p.Arg502Cys, p.Leu29Alafs*18, p.Phe252Ile, p.Val433Leu, p.Gly241Arg, p.Arg87Trp, p.Ser310Gly, p.Phe255Tyr, p.Gly416Ser, p.Arg316Cys, p.Glu9Glyfs*8, p.Arg202*, p.Gly364Arg, p.Trp432*, p.Arg398*, p.Pro305Leufs*31, p.Arg86*, p.Lys196Gln, p.Asp438Asn, p.Thr69Aspfs*12, p.Arg159Trp, p.Arg159Gln, p.Arg296Gln, p.Pro42Trpfs*7, p.Pro42TrpfsTer7, p.Leu422ProfsTer4, p.Leu422Profs*4, p.Gln239*, p.Gln239Ter, p.Thr362Ile, p.Pro138Leufs*62, p.Pro138LeufsTer62, p.His294Gln, p.Arg398Gln and p.Met1_*537del (with the other allele being non-pathogenic). In some embodiments, the subject may be heterozygous for the GBA1 gene having one allele which encodes a GCase enzyme containing one of the following amino acid variations: p.Glu365Lys, p.Thr408Met, p.Asn409Ser, p.Leu483Pro, p.Arg535His, p.Asp448His, p.Asn227Ser, p.Arg502Cys, p.Leu29Alafs*18, p.Phe252Ile, p.Val433Leu, p.Gly241Arg, p.Arg87Trp, p.Ser310Gly, p.Phe255Tyr, p.Gly416Ser, p.Arg316Cys, p.Glu9Glyfs*8, p.Arg202*, p.Gly364Arg, p.Trp432*, p.Arg398*, p.Pro305Leufs*31, p.Arg86*, p.Lys196Gln, p.Asp438Asn, p.Thr69Aspfs*12, p.Arg159Trp, p.Arg159Gln, and p.Arg296Gln (with the other allele being non-pathogenic). In addition, the subject may be homozygous for the GBA1 gene with both alleles encoding a GCase enzyme containing the amino acid variation p.Glu365Lys or p.Thr408Met.

In a further aspect of the invention, there is described Compound A, or a pharmaceutically acceptable salt thereof, for use in preventing and/or delaying clinical motor progression in a subject with Parkinson's disease who has a pathogenic variant in the glucocerebrosidase 1 (GBA1) gene (GBA-PD). According to an embodiment clinical motor progression is assessed using the Movement Disorder Society-Unified Parkinson's Disease Rating Scale (MDS-UPDRS), the Clinical Global Impression-Change (CGI-C) scale, and/or the Patient Global Impression-Change (PGI-C) scale.

In a further aspect of the invention, there is described a method for preventing and/or delaying clinical motor progression in a subject with Parkinson's disease who has a pathogenic variant in the glucocerebrosidase 1 (GBA1) gene (GBA-PD), said method comprising administering a therapeutically effective amount of Compound A, or a pharmaceutically acceptable salt thereof, to said subject. According to an embodiment, clinical motor progression is assessed using the Movement Disorder Society-Unified Parkinson's Disease Rating Scale (MDS-UPDRS), the Clinical Global Impression-Change (CGI-C) scale, and/or the Patient Global Impression-Change (PGI-C) scale.

In another aspect of the invention, there is described Compound A, or a pharmaceutically acceptable salt thereof, for use in treating Parkinson's disease in a subject who has a pathogenic variant in the glucocerebrosidase 1 (GBA1) gene (GBA-PD).

In a related aspect of the invention, there is described a method of treating Parkinson's Disease in a subject who has a pathogenic variant in the glucocerebrosidase (GBA1) gene, said method comprising administering a therapeutically effective amount of Compound A, or a pharmaceutically acceptable salt thereof, to said subject.

In a further aspect of the invention, there is described Compound A, or a pharmaceutically acceptable salt thereof, for use in preventing or delaying cognitive impairment in a subject with Parkinson's disease who has a pathogenic variant in the glucocerebrosidase 1 (GBA1) gene (GBA-PD). According to an embodiment, cognitive impairment is assessed using the Parkinson's Disease Cognitive Rating Scale (PD-CRS).

In a related aspect of the invention, there is described a method for preventing or delaying cognitive impairment in a subject with Parkinson's disease who has a pathogenic variant in the glucocerebrosidase 1 (GBA1) gene (GBA-PD), said method comprising administering a therapeutically effective amount of Compound A, or a pharmaceutically acceptable salt thereof, to said subject. According to an embodiment, cognitive impairment is assessed using Parkinson's Disease Cognitive Rating Scale (PD-CRS).

In another aspect of the present invention, there is provided Compound A, or a pharmaceutically acceptable salt thereof, for use in the treatment or prevention of Parkinson's Disease in a subject who has a pathogenic variant in the glucocerebrosidase 1 (GBA1) gene (GBA-PD) and who does not have a PD associated pathogenic variant in LRRK2.

In a related aspect of the present invention, there is provided a method for treating or preventing Parkinson's Disease in a subject with a pathogenic variant in the glucocerebrosidase 1 (GBA1) gene (GBA-PD) and who does not have a PD associated pathogenic variant in LRRK2, said method comprising administering a therapeutically effective amount of Compound A, or a pharmaceutically acceptable salt thereof, to said subject.

Pathological/pathogenic variants in the LRRK2 gene include but are not limited to G2019S, G2385R, R1628P and A419V. Pathological variants in LRRK2 are associated with inherited sporadic PD. In a preferred embodiment, the subject does not have a PD associated pathogenic variant in the LRRK2 gene selected from N1437H, R1441C, R1441H and G2019S.

A further aspect of the invention provides Compound A, or a pharmaceutically acceptable salt thereof, for use in the treatment or prevention of Parkinson's Disease in a subject who has a pathogenic variant in the glucocerebrosidase 1 (GBA1) gene (GBA-PD) and who does not suffer from Gaucher's disease.

A related aspect of the invention provides a method for treating or preventing Parkinson's Disease in a subject who has a pathogenic variant in the glucocerebrosidase 1 (GBA1) gene (GBA-PD) and who does not suffer from Gaucher's Disease, said method comprising administering a therapeutically effective amount of Compound A, or a pharmaceutically acceptable salt thereof, to said subject.

Gaucher's Disease is a genetic disorder in which glucosylceramide (GluCer) accumulates in cells and certain organs. It is thought to be caused by a hereditary deficiency of GBA1 gene. Homozygous pathogenic variants in the GBA1 gene leading to greatly reduced enzymatic activity can be the cause of GD (Clark, 2007; Cilia, 2016), which is an autosomal recessive peripheral lysosomal storage disorder arising from impaired catabolism of glycosphingolipids in the lysosome. This, in turn, elicits abnormal substrate accumulation and, thus, dysfunction in cell-signalling pathways, calcium homeostasis, and intracellular trafficking. Manifestations of GD include dysfunctions in various tissues, most notably viscera, bone, and bone marrow, and, in rare cases (types 2 and 3), brain (Cilia, 2016). In order for the disease to manifest, patients need to carry a pathogenic mutation on both alleles of the GBA gene, either in a homozygous or compound heterozygous fashion. The exception to this is when a patient is homozygous for the GBA1 mutation NM_001005741.3:c.1093G>A (E326K) or NM_001005741.3:c.1223C>T (T369M), which have been observed in GBA-PD patients but not GD patients. The GCase pathogenic variants that cause GD are those that lead to substantial reduction in enzyme activity (severe pathogenic variants), which can lead to a reduction of about 60%, of about 70%, of about 80%, of about 90% or more of the GCase activity. The identification of patients suffering from Gaucher's Disease is well known to those skilled in the art. For example, it can be diagnosed based on clinical signs and symptoms (e.g., hepatosplenomegaly, cytopenia, skeletal disease), and/or a medical history of marked deficiency of GCase activity compatible with Gaucher's Disease (for example, less than about 40%, less than about 30%, less than about 20% or less than about 10% of the GCase activity in a healthy subject or a representative sample of healthy subjects).

In another aspect, the present invention provides Compound A, or a pharmaceutically acceptable salt thereof, for use in the treatment or prevention of Parkinson's Disease in a subject who has a pathogenic variant in the glucocerebrosidase 1 (GBA1) gene (GBA-PD) and who is not homozygous for a GBA1 pathogenic variant that is known to be associated with Gaucher's Disease or compound heterozygous for two alleles of GBA1 that are known to be associated with Gaucher's Disease.

Pathogenic variants associated with Gaucher's Disease include but are not limited to heterozygous p.L483P and p.S310G mutations, and homozygous mutations of N370S, L444P, 84GG, and IVS2+1.

In some embodiments, pathogenic variants associated with subjects having Gaucher's Disease include but are not limited to subjects who are homozygous for a GBA1 pathogenic variant or are compound heterozygous having two GBA1 pathogenic variants, wherein the variant(s) is one of the following nucleotide variations: NM_001005741.3:c.1226A>G (N370S), NM_001005741.3:c.1342G>C (D409H), H255Q, D140H, G202R, L324P, 1260T, NM_001005741.3:c.1448T>C (L444P), A190T and R120W.

Further, pathogenic variants associated with subjects having Gaucher's Disease include but are not limited to subjects who are homozygous for a GBA1 gene containing one of the following nucleotide variations: NM_001005741.3:c.1226A>G, NM_001005741.3:c.1448T>C, NM_001005741.3:c.1604G>A, NM_001005741.3:c.1342G>C, NM_001005741.3:c.680A>G, NM_001005741.3:c.1504C>T, NM_001005741.3:c.84dup, NM_001005741.3:c.754T>A, NM_001005741.3:c.1297G>T, NM_001005741.3:c.721G>A, NM_001005741.3:c.259C>T, NM_001005741.3:c.928A>G, NM_001005741.3:c.764T>A, NM_001005741.3:c.1246G>A, NM_001005741.3:c.946C>T, NM_001005741.3:c.26_27del, NM_001005741.3:c.604C>T, NM_001005741.3:c.1090G>A, NM_001005741.3:c.1296G>A, NM_001005741.3:c.1192C>T, NM_001005741.3:c.914del, NM_001005741.3:c.256C>T, NM_001005741.3:c.586A>C, NM_001005741.3:c.1312G>A, NM_001005741.3:c.203dup, NM_001005741.3:c.475C>T, NM_001005741.3:c.476G>A, NM_001005741.3:c.887G>A, NM_001005741.3:c.762-1G>C, NM_001005741.3:c.115+1G>A, NM_001005741.3:c.1505+1G>T, NM_001005741.3:c.123_217del, NM_001005741.2:c.1265_1319del, NM_001005741.3:c.715C>T, NM_001005741.3:c.1085C>T, NM_001005741.3:c.413del, NM_001005741.3:c.882T>G, NM_001005741.3:c.1193G>A and NM_001005741.3:c.1_2344del. In some embodiments, pathogenic variants associated with subjects having Gaucher's Disease include but are not limited to subjects who are homozygous for a GBA1 gene containing one of the following nucleotide variations: NM_001005741.3:c.1226A>G, NM_001005741.3:c.1448T>C, NM_001005741.3:c.1604G>A, NM_001005741.3:c.1342G>C, NM_001005741.3:c.680A>G, NM_001005741.3:c.1504C>T, NM_001005741.3:c.84dup, NM_001005741.3:c.754T>A, NM_001005741.3:c.1297G>T, NM_001005741.3:c.721G>A, NM_001005741.3:c.259C>T, NM_001005741.3:c.928A>G, NM_001005741.3:c.764T>A, NM_001005741.3:c.1246G>A, NM_001005741.3:c.946C>T, NM_001005741.3:c.26_27del, NM_001005741.3:c.604C>T, NM_001005741.3:c.1090G>A, NM_001005741.3:c.1296G>A, NM_001005741.3:c.1192C>T, NM_001005741.3:c.914del, NM_001005741.3:c.256C>T, NM_001005741.3:c.586A>C, NM_001005741.3:c.1312G>A, NM_001005741.3:c.203dup, NM_001005741.3:c.475C>T, NM_001005741.3:c.476G>A, NM_001005741.3:c.887G>A, NM_001005741.3:c.762-1G>C, NM_001005741.3:c.115+1G>A, and NM_001005741.3:c.1505+1G>T. In addition, pathogenic variants associated with subjects having Gaucher's Disease include but are not limited to subjects who are compound heterozygous having two GBA1 genes containing one of the following nucleotide variations: NM_001005741.3:c.1226A>G, NM_001005741.3:c.1448T>C, NM_001005741.3:c.1604G>A, NM_001005741.3:c.1342G>C, NM_001005741.3:c.680A>G, NM_001005741.3:c.1504C>T, NM_001005741.3:c.84dup, NM_001005741.3:c.754T>A, NM_001005741.3:c.1297G>T, NM_001005741.3:c.721G>A, NM_001005741.3:c.259C>T, NM_001005741.3:c.928A>G, NM_001005741.3:c.764T>A, NM_001005741.3:c.1246G>A, NM_001005741.3:c.946C>T, NM_001005741.3:c.26_27del, NM_001005741.3:c.604C>T, NM_001005741.3:c.1090G>A, NM_001005741.3:c.1296G>A, NM_001005741.3:c.1192C>T, NM_001005741.3:c.914del, NM_001005741.3:c.256C>T, NM_001005741.3:c.586A>C, NM_001005741.3:c.1312G>A, NM_001005741.3:c.203dup, NM_001005741.3:c.475C>T, NM_001005741.3:c.476G>A, NM_001005741.3:c.887G>A, NM_001005741.3:c.762-1G>C, NM_001005741.3:c.115+1G>A, NM_001005741.3:c.1505+1G>T, NM_001005741.3:c.123_217del, NM_001005741.2:c.1265_1319del, NM_001005741.3:c.715C>T, NM_001005741.3:c.1085C>T, NM_001005741.3:c.413del, NM_001005741.3:c.882T>G, NM_001005741.3:c.1193G>A and NM_001005741.3:c.1_2344del. In some embodiments, pathogenic variants associated with subjects having Gaucher's Disease include but are not limited to subjects who are compound heterozygous having two GBA1 genes containing one of the following nucleotide variations: NM_001005741.3:c.1226A>G, NM_001005741.3:c.1448T>C, NM_001005741.3:c.1604G>A, NM_001005741.3:c.1342G>C, NM_001005741.3:c.680A>G, NM_001005741.3:c.1504C>T, NM_001005741.3:c.84dup, NM_001005741.3:c.754T>A, NM_001005741.3:c.1297G>T, NM_001005741.3:c.721G>A, NM_001005741.3:c.259C>T, NM_001005741.3:c.928A>G, NM_001005741.3:c.764T>A, NM_001005741.3:c.1246G>A, NM_001005741.3:c.946C>T, NM_001005741.3:c.26_27del, NM_001005741.3:c.604C>T, NM_001005741.3:c.1090G>A, NM_001005741.3:c.1296G>A, NM_001005741.3:c.1192C>T, NM_001005741.3:c.914del, NM_001005741.3:c.256C>T, NM_001005741.3:c.586A>C, NM_001005741.3:c.1312G>A, NM_001005741.3:c.203dup, NM_001005741.3:c.475C>T, NM_001005741.3:c.476G>A, NM_001005741.3:c.887G>A, NM_001005741.3:c.762-1G>C, NM_001005741.3:c.115+1G>A, and NM_001005741.3:c.1505+1G>T.

In a related aspect, the invention provides a method for treating or preventing Parkinson's Disease in a subject who has a pathogenic variant in the glucocerebrosidase 1 (GBA1) gene (GBA-PD) and who is not homozygous for a GBA1 pathogenic variant that is known to be associated with Gaucher's Disease or compound heterozygous for two alleles of GBA1 that are known to be associated with Gaucher's Disease, said method comprising administering a therapeutically acceptable amount of Compound A, or a pharmaceutically acceptable salt thereof, to said subject.

Examples of compound heterozygous mutations for Gaucher's Disease include p.L483P and p.S310G. Further examples of GBA1 pathogenic variants associated with Gaucher's Disease are described above.

Another aspect of the invention describes Compound A for use in preventing or limiting clinical motor progression in a subject with Parkinson's disease who has a pathogenic variant in the glucocerebrosidase 1 (GBA1) gene (GBA-PD) and who does not have a PD-associated pathogenic variant in LRRK2. According to an embodiment, clinical motor progression is assessed using the Movement Disorder Society-Unified Parkinson's Disease Rating Scale (MDS-UPDRS), the Clinical Global Impression-Change (CGI-C) scale, and/or the Patient Global Impression-Change (PGI-C) scale.

A related aspect of the invention describes a method of preventing or limiting clinical motor progression in a subject with Parkinson's disease who has a pathogenic variant in the glucocerebrosidase 1 (GBA1) gene (GBA-PD) and who does not have a PD-associated pathogenic variant in LRRK2, said method comprising administering a therapeutically acceptable amount of Compound A, or a pharmaceutically acceptable salt thereof, to said subject. According to an embodiment, clinical motor progression is assessed using the Movement Disorder Society-Unified Parkinson's Disease Rating Scale (MDS-UPDRS), the Clinical Global Impression-Change (CGI-C) scale, and/or the Patient Global Impression-Change (PGI-C) scale.

A further aspect of the invention describes Compound A for use in preventing or limiting clinical motor progression in a subject with Parkinson's disease who has a pathogenic variant in the glucocerebrosidase 1 (GBA1) gene (GBA-PD) and who does not suffer from Gaucher's disease. According to an embodiment, clinical motor progression is assessed using the Movement Disorder Society-Unified Parkinson's Disease Rating Scale (MDS-UPDRS), the Clinical Global Impression-Change (CGI-C) scale, and/or the Patient Global Impression-Change (PGI-C) scale.

A related aspect of the invention describes a method for preventing or limiting clinical motor progression in a subject with Parkinson's disease who has a pathogenic variant in the glucocerebrosidase 1 (GBA1) gene (GBA-PD) and who does not suffer from Gaucher's disease, said method comprising administering a therapeutically acceptable amount of Compound A, or a pharmaceutically acceptable salt thereof, to said subject. According to an embodiment, clinical motor progression is assessed using the Movement Disorder Society-Unified Parkinson's Disease Rating Scale (MDS-UPDRS), the Clinical Global Impression-Change (CGI-C) scale, and/or the Patient Global Impression-Change (PGI-C) scale.

Yet another aspect of the invention describes Compound A for use in preventing or limiting clinical motor progression in a subject with Parkinson's disease who has a pathogenic variant in the glucocerebrosidase 1 (GBA1) gene (GBA-PD), who does not have a PD-associated pathogenic variant in LRRK2, and who does not suffer from Gaucher's disease. According to an embodiment, clinical motor progression is assessed using the Movement Disorder Society-Unified Parkinson's Disease Rating Scale (MDS-UPDRS), the Clinical Global Impression-Change (CGI-C) scale, and/or the Patient Global Impression-Change (PGI-C) scale.

A related aspect of the invention describes a method for preventing or limiting clinical motor progression in a subject with Parkinson's disease who has a pathogenic variant in the glucocerebrosidase 1 (GBA1) gene (GBA-PD), who does not have a PD-associated pathogenic variant in LRRK2, and who does not suffer from Gaucher's disease, said method comprising administering a therapeutically acceptable amount of Compound A, or a pharmaceutically acceptable salt thereof, to said subject. According to an embodiment, clinical motor progression is assessed using the Movement Disorder Society-Unified Parkinson's Disease Rating Scale (MDS-UPDRS), the Clinical Global Impression-Change (CGI-C) scale, and/or the Patient Global Impression-Change (PGI-C) scale.

A further aspect of the invention describes Compound A for use in preventing or limiting clinical motor progression in a subject with Parkinson's disease who has a pathogenic variant in the glucocerebrosidase 1 (GBA1) gene (GBA-PD), who does not have a PD associated pathogenic variant in LRRK2, and who is not homozygous for a GBA1 pathogenic variant that is known to be associated with Gaucher's Disease or compound heterozygous for two alleles of GBA1 that are known to be associated with Gaucher's Disease. According to an embodiment, clinical motor progression is assessed using the Movement Disorder Society-Unified Parkinson's Disease Rating Scale (MDS-UPDRS), the Clinical Global Impression-Change (CGI-C) scale, and/or the Patient Global Impression-Change (PGI-C) scale.

A further aspect of the invention describes a method for preventing or limiting clinical motor progression in a subject with Parkinson's disease who has a pathogenic variant in the glucocerebrosidase 1 (GBA1) gene (GBA-PD), who does not have a PD associated pathogenic variant in LRRK2, and who is not homozygous for a GBA1 pathogenic variant that is known to be associated with Gaucher's Disease or compound heterozygous for two alleles of GBA1 that are known to be associated with Gaucher's Disease, said method comprising administering a therapeutically acceptable amount of Compound A, or a pharmaceutically acceptable salt thereof, to said subject. According to an embodiment, clinical motor progression is assessed using the Movement Disorder Society-Unified Parkinson's Disease Rating Scale (MDS-UPDRS), the Clinical Global Impression-Change (CGI-C) scale, and/or the Patient Global Impression-Change (PGI-C) scale.

Another aspect of the invention describes Compound A for use in preventing or treating Parkinson's disease in a subject who has a pathogenic variant in the glucocerebrosidase 1 (GBA1) gene (GBA-PD), who does not have a PD-associated pathogenic variant in LRRK2 and who does not have Gaucher's Disease.

Another aspect of the invention describes a method for preventing or treating Parkinson's disease in a subject who has a pathogenic variant in the glucocerebrosidase 1 (GBA1) gene (GBA-PD), who does not have a PD-associated pathogenic variant in LRRK2 and who does not have Gaucher's Disease, said method comprising administering a therapeutically acceptable amount of Compound A, or a pharmaceutically acceptable salt thereof, to said subject.

Yet another aspect of the invention describes Compound A for use in preventing or treating Parkinson's disease in a subject who has a pathogenic variant in the glucocerebrosidase 1 (GBA1) gene (GBA-PD), who does not have a PD-associated pathogenic variant in LRRK2, who does not have Gaucher's Disease, and who is not homozygous for a GBA1 pathogenic variant that is known to be associated with Gaucher's Disease or compound heterozygous for two alleles of GBA1 that are known to be associated with Gaucher's Disease.

Yet another aspect of the invention describes a method for preventing or treating Parkinson's disease in a subject who has a pathogenic variant in the glucocerebrosidase 1 (GBA1) gene (GBA-PD), who does not have a PD-associated pathogenic variant in LRRK2, who does not have Gaucher's Disease, and who is not homozygous for a GBA1 pathogenic variant that is known to be associated with Gaucher's Disease or compound heterozygous for two alleles of GBA1 that are known to be associated with Gaucher's Disease, said method comprising administering a therapeutically acceptable amount of Compound A, or a pharmaceutically acceptable salt thereof, to said subject.

A further aspect of the invention describes Compound A for use in preventing or treating Parkinson's disease in a subject who has a pathogenic variant in the glucocerebrosidase 1 (GBA1) gene (GBA-PD), who does not have Gaucher's Disease, and who is not homozygous for a GBA1 pathogenic variant that is known to be associated with Gaucher's Disease or compound heterozygous for two alleles of GBA1 that are known to be associated with Gaucher's Disease.

A further aspect of the invention describes a method for preventing or treating Parkinson's disease in a subject who has a pathogenic variant in the glucocerebrosidase 1 (GBA1) gene (GBA-PD), who does not have Gaucher's Disease, and who is not homozygous for a GBA1 pathogenic variant that is known to be associated with Gaucher's Disease or compound heterozygous for two alleles of GBA1 that are known to be associated with Gaucher's Disease, said method comprising administering a therapeutically acceptable amount of Compound A, or a pharmaceutically acceptable salt thereof, to said subject.

Another aspect of the invention describes Compound A for use in preventing or limiting clinical motor progression in a subject with Parkinson's disease who has a pathogenic variant in the glucocerebrosidase 1 (GBA1) gene (GBA-PD), who does not have Gaucher's Disease, and who is not homozygous for a GBA1 pathogenic variant that is known to be associated with Gaucher's Disease or compound heterozygous for two alleles of GBA1 that are known to be associated with Gaucher's Disease. According to an embodiment, clinical motor progression is assessed using the Movement Disorder Society-Unified Parkinson's Disease Rating Scale (MDS-UPDRS), the Clinical Global Impression-Change (CGI-C) scale, and/or the Patient Global Impression-Change (PGI-C) scale.

Another aspect of the invention describes a method for preventing or limiting clinical motor progression in a subject with Parkinson's disease who has a pathogenic variant in the glucocerebrosidase 1 (GBA1) gene (GBA-PD), who does not have Gaucher's Disease, and who is not homozygous for a GBA1 pathogenic variant that is known to be associated with Gaucher's Disease or compound heterozygous for two alleles of GBA1 that are known to be associated with Gaucher's Disease, said method comprising administering a therapeutically acceptable amount of Compound A, or a pharmaceutically acceptable salt thereof, to said subject. According to an embodiment, clinical motor progression is assessed using the Movement Disorder Society-Unified Parkinson's Disease Rating Scale (MDS-UPDRS), the Clinical Global Impression-Change (CGI-C) scale, and/or the Patient Global Impression-Change (PGI-C) scale.

Another aspect of the invention provides Compound A, or a pharmaceutically acceptable salt thereof, for use in the treatment of Parkinson's disease in a subject having decreased, reduced, or low GCase activity.

Another aspect of the invention provides a method of treating Parkinson's disease in a subject in need of such treatment and having decreased, reduced or low GCase activity, said method comprising administering a therapeutically effective amount of Compound A, or a pharmaceutically acceptable salt thereof, to said subject.

A further aspect of the invention provides Compound A, or a pharmaceutically acceptable salt thereof, for use in preventing or limiting clinical motor progression in a subject having Parkinson's disease and having decreased, reduced, or low GCase activity.

Another aspect of the invention provides a method of preventing or limiting clinical motor progression in a subject having Parkinson's disease and having decreased, reduced or low GCase activity, said method comprising administering a therapeutically effective amount of Compound A, or a pharmaceutically acceptable salt thereof, to said subject.

An aspect of the invention provides Compound A, or a pharmaceutically acceptable salt thereof, for use in preventing or delaying cognitive impairment in a subject having Parkinson's disease and having decreased, reduced, or low GCase activity. According to an embodiment, cognitive impairment is assessed using the Parkinson's Disease Cognitive Rating Scale (PD-CRS).

A related aspect of the invention provides a method of preventing or delaying cognitive impairment in a subject having Parkinson's disease and having decreased, reduced, or low GCase activity, said method comprising administering a therapeutically effective amount of Compound A, or a pharmaceutically acceptable salt thereof, to said subject. According to an embodiment, cognitive impairment is assessed using the Parkinson's Disease Cognitive Rating Scale (PD-CRS).

According to another aspect of the invention, there is described Compound A, or a pharmaceutically acceptable salt thereof, for use in preventing motor or non-motor complications, limiting clinical motor progression and/or reducing clinical motor disability in a subject having Parkinson's disease and having decreased, reduced, or low GCase activity.

In a related aspect of the invention, there is described a method for preventing motor or non-motor complications, limiting clinical motor progression and/or reducing clinical motor disability in a subject having Parkinson's disease and having decreased, reduced, or low GCase activity, said method said method comprising administering a therapeutically effective amount of Compound A, or a pharmaceutically acceptable salt thereof, to said subject.

Motor complications may be assessed using any method known to the skilled person. For example, using the MDS-UPDRS part II, III or IV. Preferably, MDS-UPDRS part IV is used to assess motor complications. Non-motor complications may be assessed using any method known to the skilled person. For example, non-motor complications may be assessed using the MDS-UPDRS part I. ‘Non-motor complications’ may also be referred to as ‘non-motor symptoms’ which can be assessed using the MDS-UPDRS part I.

In some embodiments, decreased, reduced, or low GCase activity means that the GCase activity of said patient is lower than that measured in a healthy subject or a representative sample of healthy subjects. For example, the decreased, reduced or low GCase activity may be between about 10% and about 50%, or between about 20% and about 50% below the GCase activity of a healthy subject or a representative sample of healthy subjects. In another embodiment the decreased, reduced or low GCase activity may be less than about 20%, less than about 30%, less than about 40%, less than about 50% below the GCase activity in a healthy subject or a representative sample of healthy subjects. In further embodiments, the decreased, reduced or low GCase activity may be about 10% to about 20%, about 20% to about 30%, about 30% to about 40%, or about 40% to about 50% below the GCase activity in a healthy subject or a representative sample of healthy subjects.

In some embodiments, decreased, reduced or low GCase activity means that the GCase activity is equal or comparable to the activity measured in a GBA-PD patient or a representative sample of GBA-PD patients. For example, decreased, reduced or low GCase activity may be between about 80% and about 120%, may be between about 90% and about 110%, or between about 95% and about 105% of the GCase activity seen in a GBA-PD patient or a representative sample of GBA-PD patients.

Alternatively, in some embodiments, decreased, reduced, or low GCase activity means that the GCase activity of said subject is lower than that measured in the same patient after the start of treatment, for example, once a steady state of Compound A has been reached. In some embodiments, this may be after 4, 26, 52 or 78 weeks of the start of administration of Compound A. In some embodiments, the GCase activity of the subject measured after the start of treatment is at a minimum in a steady state more than about 20% higher, more than about 30% higher, more than about 40% higher, or more than about 50% higher than the GCase activity of the subject measured before the start of treatment.

In some embodiments, the GCase activity of the subject measured after the start of treatment is at a minimum in a steady state between about 20% and about 380% higher, between about 50% and about 125% higher, or between about 69.7% and about 123% higher, than the GCase activity of the subject measured before the start of treatment, between about 150% and about 310% higher, or between about 163% and about 306% higher than that measured before the start of treatment, or between between about 250% and about 380% higher, or between about 255% and about 376% higher than that measured before the start of treatment.

In some embodiments, the GCase activity of the subject measured after the start of treatment is at a minimum in a steady state between about 50% and about 125% higher, or between about 69.7% and about 123% higher than that measured before the start of treatment, when the subject is administered 10 mg Compound A per day.

In some embodiments, the GCase activity of the subject measured after the start of treatment is at a minimum in a steady state between about about 150% and about 310% higher, or between about 163% and about 306% higher than that measured before the start of treatment, when the subject is administered 30 mg Compound A per day.

In some embodiments, the GCase activity of the subject measured after the start of treatment is at a minimum in a steady state between about 250% and about 380% higher, or between about 255% and about 376% higher than that measured before the start of treatment, when the subject is administered 60 mg Compound A per day.

Methods to determine GCase activity are known to the skilled person. Any suitable method may be used to measure the GCase activity of a subject. A summary of possible methods is described in Ysselstein, 2021. Possible methods for measuring GCase activity are summarised below in Table 1. Each method may yield slightly different results. It is therefore preferable that the same measurement methods are used when comparing GCase activities of subjects potentially eligible for treatment, GBA-PD and healthy subjects.

TABLE 1
Summary of methods that may be used to measure GCase activity.

| Assay | Substrate examples | Measures | Best applications | Disadvantages |
|---|---|---|---|---|
| Recombinant | 4-MUG, ResGlu, | GCase | Analyzing direct effects of | Does not |
| protein in | BODIPY | activity of | different | account for |
| vitro activity | glucosylceramide | recombinant | environments/compounds | variation in |
| | | protein | on GCase enzyme | endogenous |
| | | | kinetics | lysosomal |
| | | | | factors that can |
| | | | | affect activity |
| Cell lysate in | 4-MUG, ResGlu | Total GCase | Analyzing total GCase | Is not able to |
| vitro activity | BODIPY | protein that | protein, the effect of | correct for |
| | glucosylceramide | includes | GCase mutations and | difference in |
| | | lysosomal | covalent modification on | GCase levels, |
| | | and | GCase activity | which affect |
| | | nonlysosomal | | measured |
| | | GCase | | activity |
| Patient | 4-MUG, ResGlu | Total GCase | Activity measurement in | Function of |
| biofluid in | | protein | serum and CSF | GCase in serum |
| vitro activity | | | | and CSF and |
| | | | | correlation with |
| | | | | tissue activity is |
| | | | | unknown |
| Inhibody | MDW333, | Lysosomal | Quantifying lysosomal | Quantifies |
| | MDW941 | GCase | GCase protein, analyzing | levels of active |
| | | protein | GCase protein by | protein not |
| | | | microscopy | enzyme activity |
| In situ | PFB-FDGlu | In situ | Analyzing lysosomal | Measurement |
| GCase | | lysosomal | GCase activity while | will be affected |
| activity-cell | | GCase | accounting for | by differences |
| culture | | activity | endogenous factors | in substrate |
| | | | | uptake |
| In situ | PFB-FDGlu | In situ | Analyzing lysosomal | Measurement |
| GCase | | lysosomal | GCase activity while | will be affected |
| activity- | | GCase | accounting for | by differences |
| PBMC | | activity | endogenous factors | in substrate |
| | | | | uptake |
| Dry blood | C12 | Total GCase | Analyzing total GCase | Requires |
| spot assay | glucosylceramide | protein that | protein, the effect of | specialized |
| | | includes | GCase mutations, | sample |
| | | lysosomal | covalent modification on | preparation and |
| | | and | GCase activity | equipment; |
| | | nonlysosomal | | does not |
| | | GCase | | account for |
| | | | | variation in |
| | | | | endogenous |
| | | | | lysosomal |
| | | | | factors |

Another aspect of the invention provides Compound A, or a pharmaceutically acceptable salt thereof, for use in the treatment of Parkinson's disease in a subject having altered lysosomal activity.

Another aspect of the invention provides a method of treating Parkinson's disease in a subject in need of such treatment and having altered lysosomal activity, said method comprising administering a therapeutically effective amount of Compound A, or a pharmaceutically acceptable salt thereof, to said subject.

A further aspect of the invention provides Compound A, or a pharmaceutically acceptable salt thereof, for use in preventing or limiting clinical motor progression in a subject having Parkinson's disease and having altered lysosomal activity.

Another aspect of the invention provides a method of preventing or limiting clinical motor progression in a subject having Parkinson's disease and having altered lysosomal activity, said method comprising administering a therapeutically effective amount of Compound A, or a pharmaceutically acceptable salt thereof, to said subject.

An aspect of the invention provides Compound A, or a pharmaceutically acceptable salt thereof, for use in preventing or delaying cognitive impairment in a subject having Parkinson's disease and having altered lysosomal activity. According to an embodiment, cognitive impairment is assessed using the Parkinson's Disease Cognitive Rating Scale (PD-CRS).

A related aspect of the invention provides a method of preventing or delaying cognitive impairment in a subject having Parkinson's disease and having altered lysosomal activity, said method comprising administering a therapeutically effective amount of Compound A, or a pharmaceutically acceptable salt thereof, to said subject. According to an embodiment, cognitive impairment is assessed using the Parkinson's Disease Cognitive Rating Scale (PD-CRS).

According to another aspect of the invention, there is described Compound A, or a pharmaceutically acceptable salt thereof, for use in preventing motor or non-motor complications, limiting clinical motor progression and/or reducing clinical motor disability in a subject having Parkinson's disease and having altered lysosomal activity.

In a related aspect of the invention, there is described a method for preventing motor or non-motor complications, limiting clinical motor progression and/or reducing clinical motor disability in a subject having Parkinson's disease and having altered lysosomal activity, said method said method comprising administering a therapeutically effective amount of Compound A, or a pharmaceutically acceptable salt thereof, to said subject.

Methods to determine lysosomal activity are known to the skilled person. Any suitable method may be used to measure the lysosomal activity of a subject. In some embodiments, modulated levels of GCase substrates glucosylceramide (GluCer) and/or glucosylsphingosine (GluSph) can be used to determine lysosomal activity, particularly altered lysosomal activity. For example, GluSph has been described as a clinically relevant marker of GBA-PD (Leyns et al. 2023; Surface et al. 2022); and it has been shown in GBA-PD patients that GluSph levels are increased compared to healthy controls (Pires et al. 2023).

According to an embodiment of the above aspects of the invention, the Parkinson's disease patient has increased or high levels of GluSph before start of treatment with Compound A, or a pharmaceutically acceptable salt thereof.

Increased or high levels of GluSph means that the GluSph levels of said patient are higher than those measured in a healthy subject or a representative sample of healthy subjects. Alternatively, increased or high levels of GluSph means that the GluSph levels of said patient before start of treatment (e.g. at baseline) are higher than those measured in the same patient following prolonged administration of Compound A, or a pharmaceutically acceptable salt thereof (e.g. following 12, 26, 39, 52, 65, 78 weeks or longer of once-daily administration of a therapeutically effective amount of Compound A, or a pharmaceutically acceptable salt thereof).

According to an embodiment of the above aspects of the invention, administration of a therapeutically effective amount of Compound A, or a pharmaceutically acceptable salt thereof reduces the levels of GluSph in a patient suffering from Parkinson's disease, for example GBA-PD.

Reducing the levels of GluSph means that the GluSph levels of a patient are lower following prolonged administration of Compound A, or a pharmaceutically acceptable salt thereof (e.g. following 12, 26, 39, 52, 65, 78 weeks or longer of once-daily administration of a therapeutically effective amount of Compound A, or a pharmaceutically acceptable salt thereof), than those measured in the same patient before administration of Compound A or a pharmaceutically acceptable salt thereof (e.g. at baseline).

Moreover, baseline levels of GluSph and/or GluCer could be used as baseline predictors of treatment response to prolonged administration of Compound A, or a pharmaceutically acceptable salt thereof (e.g. following 12, 26, 39, 52, 65, 78 weeks or longer, of once-daily administration of a therapeutically effective amount of Compound A, or a pharmaceutically acceptable salt thereof), for example, patient response may be dependent on the levels of GluSph and/or GluCer at baseline.

Methods to measure both GluCer and GluSph are described in Heijer et al. (2021) which is incorporated herein in its entirety. Briefly: GluCer and GluSph can be measured in K₂EDTA plasma using a LC-MS/MS method. The carbon chain of a ceramide group like in GluCer can be of varying length and saturation. Concentrations were measured of GluCer C16:0, C18:0, C22:0, C24:0 and C24:1. For GluCer the assay range was 1.00-2500 pmol. For GluSph the assay range was 0.0500-10.0 pmol.

Possible methods for measuring lysosomal activity may yield slightly different results. It is therefore preferable that the same measurement methods are used when comparing lysosomal activities of subjects potentially eligible for treatment, GBA-PD and/or healthy subjects.

Another aspect of the invention provides Compound A for use in preventing or reducing the risk of Parkinson's disease in a subject determined as being at risk of Parkinson's disease, for example a subject not diagnosed as having Parkinson's disease and determined as carrying a GBA1 pathogenic variant for PD.

A related aspect of the invention provides a method for preventing or reducing the risk of Parkinson's disease in a subject determined as being at risk of Parkinson's disease, for example a subject not diagnosed as having Parkinson's disease and determined as carrying a GBA1 pathogenic variant for PD, said method comprising administering a therapeutically effective amount of Compound A, or a pharmaceutically acceptable salt thereof, to said subject.

Another aspect of the invention describes Compound A, or a pharmaceutically acceptable salt thereof, for use in for preventing or delaying cognitive impairment in a subject with Parkinson's disease who has a pathogenic variant in the glucocerebrosidase 1 (GBA1) gene (GBA-PD) and who does not have a PD-associated pathogenic variant in LRRK2. According to an embodiment, cognitive impairment is assessed using Parkinson's Disease Cognitive Rating Scale (PD-CRS).

A related aspect of the invention describes a method of preventing or delaying cognitive impairment in a subject with Parkinson's disease who has a pathogenic variant in the glucocerebrosidase 1 (GBA1) gene (GBA-PD) and who does not have a PD-associated pathogenic variant in LRRK2, said method comprising administering a therapeutically acceptable amount of Compound A, or a pharmaceutically acceptable salt thereof, to said subject. According to an embodiment, cognitive impairment is assessed using Parkinson's Disease Cognitive Rating Scale (PD-CRS).

A further aspect of the invention describes Compound A, or a pharmaceutically acceptable salt thereof, for use in preventing or delaying cognitive impairment in a subject with Parkinson's disease who has a pathogenic variant in the glucocerebrosidase 1 (GBA1) gene (GBA-PD) and who does not suffer from Gaucher's disease. According to an embodiment, cognitive impairment is assessed using Parkinson's Disease Cognitive Rating Scale (PD-CRS).

A related aspect of the invention describes a method for preventing or delaying cognitive impairment in a subject with Parkinson's disease who has a pathogenic variant in the glucocerebrosidase 1 (GBA1) gene (GBA-PD) and who does not suffer from Gaucher's disease, said method comprising administering a therapeutically acceptable amount of Compound A, or a pharmaceutically acceptable salt thereof, to said subject. According to an embodiment, cognitive impairment is assessed using Parkinson's Disease Cognitive Rating Scale (PD-CRS).

Yet another aspect of the invention describes Compound A, or a pharmaceutically acceptable salt thereof, for use in preventing or delaying cognitive impairment in a subject with Parkinson's disease who has a pathogenic variant in the glucocerebrosidase 1 (GBA1) gene (GBA-PD), who does not have a PD-associated pathogenic variant in LRRK2, and who does not suffer from Gaucher's disease. According to an embodiment, cognitive impairment is assessed using Parkinson's Disease Cognitive Rating Scale (PD-CRS).

A related aspect of the invention describes a method for preventing or delaying cognitive impairment in a subject with Parkinson's disease who has a pathogenic variant in the glucocerebrosidase 1 (GBA1) gene (GBA-PD), who does not have a PD-associated pathogenic variant in LRRK2, and who does not suffer from Gaucher's disease, said method comprising administering a therapeutically acceptable amount of Compound A, or a pharmaceutically acceptable salt thereof, to said subject. According to an embodiment, cognitive impairment is assessed using Parkinson's Disease Cognitive Rating Scale (PD-CRS).

A further aspect of the invention describes Compound A, or a pharmaceutically acceptable salt thereof, for use in preventing or delaying cognitive impairment in a subject with Parkinson's disease who has a pathogenic variant in the glucocerebrosidase 1 (GBA1) gene (GBA-PD), who does not have a PD associated pathogenic variant in LRRK2, and who is not homozygous for a GBA1 pathogenic variant that is known to be associated with Gaucher's Disease or compound heterozygous for two alleles of GBA1 that are known to be associated with Gaucher's Disease. According to an embodiment, cognitive impairment is assessed using Parkinson's Disease Cognitive Rating Scale (PD-CRS).

A further aspect of the invention describes a method for preventing or delaying cognitive impairment in a subject with Parkinson's disease who has a pathogenic variant in the glucocerebrosidase 1 (GBA1) gene (GBA-PD), who does not have a PD associated pathogenic variant in LRRK2, and who is not homozygous for a GBA1 pathogenic variant that is known to be associated with Gaucher's Disease or compound heterozygous for two alleles of GBA1 that are known to be associated with Gaucher's Disease, said method comprising administering a therapeutically acceptable amount of Compound A, or a pharmaceutically acceptable salt thereof, to said subject. According to an embodiment, cognitive impairment is assessed using Parkinson's Disease Cognitive Rating Scale (PD-CRS).

According to another aspect of the invention, there is described Compound A, or a pharmaceutically acceptable salt thereof, for use in preventing motor or non-motor complications, limiting clinical motor progression and/or improving clinical motor disability in a subject with Parkinson's disease who has a pathogenic variant in the glucocerebrosidase 1 (GBA1) gene (GBA-PD).

A related aspect of the invention provides a method for treating motor or non-motor complications, limiting clinical motor progression and/or reducing clinical motor disability in a subject with Parkinson's disease who has a pathogenic variant in glucocerebrosidase 1 (GBA1) gene (GBA-PD), said method comprising administering a therapeutically effective amount of Compound A, or a pharmaceutically acceptable salt thereof, to said subject.

Another aspect of the invention provides Compound A for use in treating motor or non-motor complications, limiting clinical motor progression and/or reducing clinical motor disability in a subject with Parkinson's disease who has a pathogenic variant in glucocerebrosidase 1 (GBA1) gene (GBA-PD).

Another aspect of the invention describes Compound A, or a pharmaceutically acceptable salt thereof, for use in treating motor or non-motor complications, limiting clinical motor progression and/or reducing clinical motor disability in a subject with Parkinson's disease who has a pathogenic variant in the glucocerebrosidase 1 (GBA1) gene (GBA-PD) and who does not have a PD-associated pathogenic variant in LRRK2.

A related aspect of the invention describes a method of treating motor or non-motor complications, limiting clinical motor progression and/or reducing clinical motor disability in a subject with Parkinson's disease who has a pathogenic variant in the glucocerebrosidase 1 (GBA1) gene (GBA-PD) and who does not have a PD-associated pathogenic variant in LRRK2, said method comprising administering a therapeutically acceptable amount of Compound A, or a pharmaceutically acceptable salt thereof, to said subject.

A further aspect of the invention describes Compound A, or a pharmaceutically acceptable salt thereof, for use in treating motor or non-motor complications, limiting clinical motor progression and/or reducing clinical motor disability in a subject with Parkinson's disease who has a pathogenic variant in the glucocerebrosidase 1 (GBA1) gene (GBA-PD) and who does not suffer from Gaucher's disease.

A related aspect of the invention describes a method for treating motor or non-motor complications, limiting clinical motor progression and/or reducing clinical motor disability in a subject with Parkinson's disease who has a pathogenic variant in the glucocerebrosidase 1 (GBA1) gene (GBA-PD) and who does not suffer from Gaucher's disease, said method comprising administering a therapeutically acceptable amount of Compound A, or a pharmaceutically acceptable salt thereof, to said subject.

Yet another aspect of the invention describes Compound A, or a pharmaceutically acceptable salt thereof, for use in treating motor or non-motor complications, limiting clinical motor progression and/or reducing clinical motor disability in a subject with Parkinson's disease who has a pathogenic variant in the glucocerebrosidase 1 (GBA1) gene (GBA-PD), who does not have a PD-associated pathogenic variant in LRRK2, and who does not suffer from Gaucher's disease.

A related aspect of the invention describes a method for treating motor or non-motor complications, limiting clinical motor progression and/or reducing clinical motor disability in a subject with Parkinson's disease who has a pathogenic variant in the glucocerebrosidase 1 (GBA1) gene (GBA-PD), who does not have a PD-associated pathogenic variant in LRRK2, and who does not suffer from Gaucher's disease, said method comprising administering a therapeutically acceptable amount of Compound A, or a pharmaceutically acceptable salt thereof, to said subject.

A further aspect of the invention describes Compound A, or a pharmaceutically acceptable salt thereof, for use in treating motor or non-motor complications, limiting clinical motor progression and/or reducing clinical motor disability in a subject with Parkinson's disease who has a pathogenic variant in the glucocerebrosidase 1 (GBA1) gene (GBA-PD), who does not have a PD associated pathogenic variant in LRRK2, and who is not homozygous for a GBA1 pathogenic variant that is known to be associated with Gaucher's Disease or compound heterozygous for two alleles of GBA1 that are known to be associated with Gaucher's Disease.

A further aspect of the invention describes a method for treating motor or non-motor complications, limiting clinical motor progression and/or reducing clinical motor disability in a subject with Parkinson's disease who has a pathogenic variant in the glucocerebrosidase 1 (GBA1) gene (GBA-PD), who does not have a PD associated pathogenic variant in LRRK2, and who is not homozygous for a GBA1 pathogenic variant that is known to be associated with Gaucher's Disease or compound heterozygous for two alleles of GBA1 that are known to be associated with Gaucher's Disease, said method comprising administering a therapeutically acceptable amount of Compound A, or a pharmaceutically acceptable salt thereof, to said subject.

Motor complications may be assessed using any method known to the skilled person. For example, using the MDS-UPDRS part II, III or IV. Preferably, MDS-UPDRS part IV is used to assess motor complications. Non-motor complications may be assessed using any method known to the skilled person. For example, non-motor complications may be assessed using the MDS-UPDRS part I. ‘Non-motor complications’ may also be referred to as ‘non-motor symptoms’ which can be assessed using the MDS-UPDRS part I.

In another aspect of the invention, there is described Compound A, or a pharmaceutically acceptable salt thereof, for use in preventing or limiting quality of life deterioration, and/or improving quality of life, in a subject with Parkinson's disease who has a pathogenic variant in the glucocerebrosidase 1 (GBA1) gene (GBA-PD).

A related aspect of the invention provides a method for preventing or limiting quality of life deterioration, and/or improving quality of life, in a subject with Parkinson's disease who has a pathogenic variant in the glucocerebrosidase 1 (GBA1) gene (GBA-PD), said method comprising administering a therapeutically effective amount of Compound A, or a pharmaceutically acceptable salt thereof, to said subject.

Another aspect of the invention describes Compound A, or a pharmaceutically acceptable salt thereof, for use in preventing or limiting quality of life deterioration, and/or improving quality of life, in a subject with Parkinson's disease who has a pathogenic variant in the glucocerebrosidase 1 (GBA1) gene (GBA-PD) and who does not have a PD-associated pathogenic variant in LRRK2.

A related aspect of the invention describes a method of preventing or limiting quality of life deterioration, and/or improving quality of life, in a subject with Parkinson's disease who has a pathogenic variant in the glucocerebrosidase 1 (GBA1) gene (GBA-PD) and who does not have a PD-associated pathogenic variant in LRRK2, said method comprising administering a therapeutically acceptable amount of Compound A, or a pharmaceutically acceptable salt thereof, to said subject.

A further aspect of the invention describes Compound A, or a pharmaceutically acceptable salt thereof, for use in preventing or limiting quality of life deterioration, and/or improving quality of life, in a subject with Parkinson's disease who has a pathogenic variant in the glucocerebrosidase 1 (GBA1) gene (GBA-PD) and who does not suffer from Gaucher's disease.

A related aspect of the invention describes a method for preventing or limiting quality of life deterioration, and/or improving quality of life, in a subject with Parkinson's disease who has a pathogenic variant in the glucocerebrosidase 1 (GBA1) gene (GBA-PD) and who does not suffer from Gaucher's disease, said method comprising administering a therapeutically acceptable amount of Compound A, or a pharmaceutically acceptable salt thereof, to said subject.

Yet another aspect of the invention describes Compound A, or a pharmaceutically acceptable salt thereof, for use in preventing or limiting quality of life deterioration, and/or improving quality of life, in a subject with Parkinson's disease who has a pathogenic variant in the glucocerebrosidase 1 (GBA1) gene (GBA-PD), who does not have a PD-associated pathogenic variant in LRRK2, and who does not suffer from Gaucher's disease.

A related aspect of the invention describes a method for preventing or limiting quality of life deterioration, and/or improving quality of life, in a subject with Parkinson's disease who has a pathogenic variant in the glucocerebrosidase 1 (GBA1) gene (GBA-PD), who does not have a PD-associated pathogenic variant in LRRK2, and who does not suffer from Gaucher's disease, said method comprising administering a therapeutically acceptable amount of Compound A, or a pharmaceutically acceptable salt thereof, to said subject.

A further aspect of the invention describes Compound A, or a pharmaceutically acceptable salt thereof, for use in preventing or limiting quality of life deterioration, and/or improving quality of life, in a subject with Parkinson's disease who has a pathogenic variant in the glucocerebrosidase 1 (GBA1) gene (GBA-PD), who does not have a PD associated pathogenic variant in LRRK2, and who is not homozygous for a GBA1 pathogenic variant that is known to be associated with Gaucher's Disease or compound heterozygous for two alleles of GBA1 that are known to be associated with Gaucher's Disease.

A further aspect of the invention describes a method for preventing or limiting quality of life deterioration, and/or improving quality of life, in a subject with Parkinson's disease who has a pathogenic variant in the glucocerebrosidase 1 (GBA1) gene (GBA-PD), who does not have a PD associated pathogenic variant in LRRK2, and who is not homozygous for a GBA1 pathogenic variant that is known to be associated with Gaucher's Disease or compound heterozygous for two alleles of GBA1 that are known to be associated with Gaucher's Disease, said method comprising administering a therapeutically acceptable amount of Compound A, or a pharmaceutically acceptable salt thereof, to said subject.

Quality of life may be assessed using any method known to the skilled person. For example, using the EuroQol 5 Dimension 5 Level (EQ-5D-5L) score and/or the Parkinson's Disease Questionnaire (PDQ-39).

The PDQ-39 is the most thoroughly validated and extensively used self-report measure for the assessment of health-related quality of life in patients with PD. The EQ-5D-5L is a patient-reported outcome that measures health in 5 dimensions.

In an embodiment, the effect on quality of life of said use or treatment as described in any of the above aspects is assessed using the 39-item Parkinson's Disease questionnaire (PDQ-39) score, and/or the EuroQol 5 Dimension 5 Level (EQ-5D-5L) score.

In an embodiment, said use or treatment as described in any of the aspects above results in increased time for a 2-point increase in the MDS-UPDRS Part II score and no improvement (i.e., score of zero or higher) in the Part III score compared to a subject treated with a placebo. In another embodiment, said use or treatment as described in any of the aspects above results in an increase in time from baseline to clinically meaningful progression on motor aspects of experiences of daily living, as assessed by 2-point increase in the MDS UPDRS Part II score and no improvement (i.e., difference from baseline of zero or higher) in the Part III score compared to a subject treated with a placebo.

In an embodiment, said use or treatment as described in any of the aspects described above results in increased time for a 5-point increase in the MDS-UPDRS Part III total score compared to a subject treated with a placebo.

The MDS-UPDRS score ranges from 0-132 with 32 and below being mild and 59 and above being severe. An increase of greater than or equal to 5 points in the MDS-UPDRS Part III is considered a measurable degradation in motor function.

In an embodiment, said use or treatment as described in any of the aspects above results in increased time for a 3-point increase in the MDS-UPDRS Part III total score compared to a subject treated with a placebo considered as a potential minimum measure of degradation in motor function.

In an embodiment, said use or treatment as described in any of the aspects above results in increased time for a 2-point increase in the MDS-UPDRS Part II score and confirmed by >5-point increase in the MDS-UPDRS Part III score compared to a subject treated with a placebo.

In an embodiment, said use or treatment as described in any of the aspects above results in increased time for any worsening on at least one measure selected from the CGI-C, PGI-C, CGI-S, or PGI-S compared to a subject treated with a placebo.

In an embodiment, said use or treatment as described above results in increased time, compared to a subject treated with a placebo, until:
i) first levodopa-equivalent daily dosage increase (LEDD);
ii) any worsening on the Clinical Global Impression-Change (CGI-C) scale;
iii) any worsening on the Patient Global Impression-Change (PGI-C) scale;
iv) any worsening on the Clinical Global Impression-Severity (CGI-S) scale; or
v) any worsening on the Patient Global Impression-Severity (PGI-S) scale.

In an embodiment, said use or treatment as described above results in a decreased change from baseline for one or more of the below scales compared to a subject treated with placebo:
i) MDS-UPDRS Total (Part I-IV) score;
ii) MDS-UPDRS Part I score;
iii) MDS-UPDRS Part II score;
iv) MDS-UPDRS Part III score;
v) MDS-UPDRS Part IV score;
vi) MDS-UPDRS Part II+Part III score;
vii) Bradykinesia as measured by the MDS-UPDRS Part III Global Spontaneity of Movement;
viii) Modified Hoehn and Yahr score; and/or
ix) PD-CRS score

In an embodiment, said use or treatment as described above results in an improved gait speed compared to a subject treated with a placebo. In some embodiments, said use or treatment as described above results in preventing, limiting or delaying a decline in gait speed.

Motor symptoms such as bradykinesia, postural instability, rest tremor, rigidity, and slowness of movement are usually present in PD. These symptoms promote alteration in gait parameters. Self-selected walking speed (SSWS) is reduced in people with PD. Notably, individuals with PD walk with higher cadence, shorter stride length and have a higher time in double limb support phase.

Improved gait speed is therefore indicative of an improvement in clinical motor function.

Gait speed may be measured using any method known to the skilled person. For example, pedometers, or “Smart” watches.

In an embodiment, said use or treatment as described above results in improved cerebral blood flow as measured using arterial spin labelling and/or MRI free-water imaging.

Abnormalities in the regulation of the cardiovascular system due to autonomic nervous system (ANS) dysfunction may lead to a sudden decline in blood pressure (BP) upon standing, sitting or performing activities/exercises in patients with Parkinson's Disease. Cerebral blood flow, or perfusion, is a measure of the rate of delivery of arterial blood to a capillary bed in tissue and indicative of cardiovascular health.

In an embodiment, said use or treatment as described in any of the above aspects results in reduced neurofilament light chain concentrations.

Neurofilament light chain (NfL) is a neuronal cytoplasmic protein highly expressed in large calibre myelinated axons. Its levels increase in cerebrospinal fluid (CSF) and blood proportionally to the degree of axonal damage in neurodegenerative diseases and Parkinson's disease. New immunoassays able to detect biomarkers at ultralow levels have allowed for the measurement of NfL in blood, thus making it possible to easily and repeatedly measure NfL for monitoring the course of Parkinson's Disease.

In an embodiment, said use or treatment as described in any of the above aspects comprises administering to the subject a dose of about 10 mg of Compound A per day.

In an embodiment, said use or treatment as described in any of the above aspects comprises administering to the subject a dose of about 30 mg of Compound A per day.

In an embodiment, said use or treatment as described in any of the above aspects comprises administering to the subject a dose of about 60 mg of Compound A per day.

In an embodiment, said use or treatment as described in any of the above aspects results in a minimum activation of GCase activity of the subject at steady state between about 50% and about 125%, or between about 69.7% and about 123%, when the subject is administered 10 mg Compound A per day.

In an embodiment, said use or treatment as described in any of the above aspects results in a minimum activation of GCase activity of the subject at steady state between about 150% and about 310%, or between about 163% and about 306%, when the subject is administered 30 mg Compound A per day.

In an embodiment, said use or treatment as described in any of the above aspects results in a minimum activation of GCase activity of the subject at steady state between about 250% and about 380%, or between about 255% and about 376%, when the subject is administered 60 mg Compound A per day.

In an embodiment, said use or treatment as described in any of the above aspects results in a minimum activation of GCase activity of the subject at steady state of more than about 20%, more than about 30%, more than about 40%, or more than about 50%.

In an embodiment, said use or treatment as described in any of the above aspects results in a minimum activation of GCase activity of the subject at steady state of between about 20% and about 380%, between about 50% and about 125%, between about 69.7% and about 123%, between about 150% and about 310%, between about 163% and about 306%, between between about 250% and about 380%, or between about 255% and about 376%.

The GCase activity may be measured by any of the methods discussed above. Suitable methods include measurement of GCase Plasmatic Activity or GCase whole blood activity.

In any of the aspects above, Compound A, or a pharmaceutically acceptable salt thereof, can be used or administered to a subject for a prolonged period of time. A prolonged period of time can be for 12 or more weeks, 26 or more weeks, 39 or more weeks, 52 or more weeks, 65 or more weeks, or 78 or more weeks.

Preferably, in any of the aspects or embodiments described herein, Compound A is administered once-daily. Preferably, Compound A is administered orally.

In an embodiment, said use or treatment as described in any of the above aspects comprises administering to a subject who is a carrier of at least one major/severe GBA1 mutation, for example, a GBA1 mutation heterozygous for N370S, D409H, H255Q, D140H, G202R, L324P, I260T, L444P, A190T or R120W, or homozygous for T369M or E326K.

In an embodiment, said use or treatment as described in any of the above aspects comprises administering to a subject who is a carrier of at least one minor/mild GBA1 mutation, for example, a GBA1 mutation heterozygous for T369M or E326K.

Further, said use or treatment as described in any of the above aspects may comprise administering to a subject who is a carrier of at least one pathological/pathogenic GBA1 mutation, for example, selected from one of the following nucleotide variations: NM_001005741.3:c.1093G>A, NM_001005741.3:c.1223C>T, NM_001005741.3:c.1226A>G, NM_001005741.3:c.1448T>C, NM_001005741.3:c.1604G>A, NM_001005741.3:c.1342G>C, NM_001005741.3:c.680A>G, NM_001005741.3:c.1504C>T, NM_001005741.3:c.84dup, NM_001005741.3:c.754T>A, NM_001005741.3:c.1297G>T, NM_001005741.3:c.721G>A, NM_001005741.3:c.259C>T, NM_001005741.3:c.928A>G, NM_001005741.3:c.764T>A, NM_001005741.3:c.1246G>A, NM_001005741.3:c.946C>T, NM_001005741.3:c.26_27del, NM_001005741.3:c.604C>T, NM_001005741.3:c.1090G>A, NM_001005741.3:c.1296G>A, NM_001005741.3:c.1192C>T, NM_001005741.3:c.914del, NM_001005741.3:c.256C>T, NM_001005741.3:c.586A>C, NM_001005741.3:c.1312G>A, NM_001005741.3:c.203dup, NM_001005741.3:c.475C>T, NM_001005741.3:c.476G>A, NM_001005741.3:c.887G>A, NM_001005741.3:c.762-1G>C, NM_001005741.3:c.115+1G>A, NM_001005741.3:c.1505+1G>T, NM_001005741.3:c.123_217del, NM_001005741.2:c.1265_1319del, NM_001005741.3:c.715C>T, NM_001005741.3:c.1085C>T, NM_001005741.3:c.413del, NM_001005741.3:c.882T>G, NM_001005741.3:c.1193G>A and NM_001005741.3:c.1_2344del. In some embodiments, the at least one pathological/pathogenic GBA1 mutation may be selected from one of the following nucleotide variations: NM_001005741.3:c.1093G>A, NM_001005741.3:c.1223C>T, NM_001005741.3:c.1226A>G, NM_001005741.3:c.1448T>C, NM_001005741.3:c.1604G>A, NM_001005741.3:c.1342G>C, NM_001005741.3:c.680A>G, NM_001005741.3:c.1504C>T, NM_001005741.3:c.84dup, NM_001005741.3:c.754T>A, NM_001005741.3:c.1297G>T, NM_001005741.3:c.721G>A, NM_001005741.3:c.259C>T, NM_001005741.3:c.928A>G, NM_001005741.3:c.764T>A, NM_001005741.3:c.1246G>A, NM_001005741.3:c.946C>T, NM_001005741.3:c.26_27del, NM_001005741.3:c.604C>T, NM_001005741.3:c.1090G>A, NM_001005741.3:c.1296G>A, NM_001005741.3:c.1192C>T, NM_001005741.3:c.914del, NM_001005741.3:c.256C>T, NM_001005741.3:c.586A>C, NM_001005741.3:c.1312G>A, NM_001005741.3:c.203dup, NM_001005741.3:c.475C>T, NM_001005741.3:c.476G>A, NM_001005741.3:c.887G>A, NM_001005741.3:c.762-1G>C, NM_001005741.3:c.115+1G>A, and NM_001005741.3:c.1505+1G>T.

Alternatively, said use or treatment as described in any of the above aspects may comprise administering to a subject who is a carrier of at least one pathological/pathogenic GBA1 mutation in the amino acid sequence of the GCase enzyme selected from p.Glu365Lys, p.Thr408Met, p.Asn409Ser, p.Leu483Pro, p.Arg535His, p.Asp448His, p.Asn227Ser, p.Arg502Cys, p.Leu29Alafs*18, p.Phe252Ile, p.Val433Leu, p.Gly241Arg, p.Arg87Trp, p.Ser310Gly, p.Phe255Tyr, p.Gly416Ser, p.Arg316Cys, p.Glu9Glyfs*8, p.Arg202*, p.Gly364Arg, p.Trp432*, p.Arg398*, p.Pro305Leufs*31, p.Arg86*, p.Lys196Gln, p.Asp438Asn, p.Thr69Aspfs*12, p.Arg159Trp, p.Arg159Gln, p.Arg296Gln, p.Pro42Trpfs*7, p.Pro42TrpfsTer7, p.Leu422ProfsTer4, p.Leu422Profs*4, p.Gln239*, p.Gln239Ter, p.Thr362Ile, p.Pro138Leufs*62, p.Pro138LeufsTer62, p.His294Gln, p.Arg398Gln and p.Met1_*537del. In some embodiments, the at least one pathological/pathogenic GBA1 mutation in the amino acid sequence of the GCase enzyme may be selected from p.Glu365Lys, p.Thr408Met, p.Asn409Ser, p.Leu483Pro, p.Arg535His, p.Asp448His, p.Asn227Ser, p.Arg502Cys, p.Leu29Alafs*18, p.Phe252Ile, p.Val433Leu, p.Gly241Arg, p.Arg87Trp, p.Ser310Gly, p.Phe255Tyr, p.Gly416Ser, p.Arg316Cys, p.Glu9Glyfs*8, p.Arg202*, p.Gly364Arg, p.Trp432*, p.Arg398*, p.Pro305Leufs*31, p.Arg86*, p.Lys196Gln, p.Asp438Asn, p.Thr69Aspfs*12, p.Arg159Trp, p.Arg159Gln, and p.Arg296Gln.

Further information on the subject groups to which Compound A can be administered, for example, with regard to the hetero/homozygous nature of the pathological/pathogenic GBA1 variants, can be found above.

In some embodiments, said use or treatment as described in any of the above aspects comprises administering to a subject who has a clinical diagnosis of PD for at least 1 year and for no longer than 7 years as confirmed by using the MDS Criteria for Parkinson's Disease.

In some embodiments, said use or treatment as described in any of the above aspects comprises administering to a subject who has a modified Hoehn and Yahr score of 52.5.

In some embodiments, said use or treatment as described in any of the above aspects comprises administering to a subject who has a score of 22 on the MoCA scale.

In some embodiments, said use or treatment as described in any of the above aspects comprises administering to a subject who does not have moderate (or severe) motor complications as assessed by a score ≥3 in any of the subitems of the MDS-UPDRS Part IV.

In some embodiments, the subject has no, slight or mild motor complications as assessed by a score <3, such as a score of 0, 1 or 2, in any of the subitems of the MDS-UPDRS Part IV.

In some embodiments, said use or treatment as described in any of the above aspects comprises administering to a subject who does not have clinically significant psychosis.

Compound A may be administered in combination with a therapeutically effective amount of one or more of the following concomitant drugs: Dopaminergic agents (for example, Levodopa, Levodopa/DOPA decarboxylase inhibitor (DDCI) preparations, such as Levodopa/Carbidopa, Levodopa/Benserazide and Levodopa/Carbidopa/entacapone. Said levodopa or levodopa preparations can be provided as immediate-release, controlled-release or extended-release formulations), Dopamine receptor agonists (for example, Pramipexole, Ropinirole, Rotigotine), Monoamine oxidase B inhibitors (for example, Rasagiline, Safinamide, Selegiline), Catechol-O-methyltransferase inhibitors (for example, Entacapone, Tolcapone, Opicapone), N methyl-D-Aspartate Receptor antagonists (for example, Amantadine), Adenosine receptor antagonists (for example, Istradefylline), Anticholinergic agents (for example Benztropine, Biperiden, Trihexyphenidyl), and neuroprotective agents (for example, Ambroxol).

When using such a combination, the administration time of the Compound A and the concomitant drug is not restricted, and Compound A or a pharmaceutical composition thereof, or the concomitant drug or a pharmaceutical composition thereof can be administered to an administration subject simultaneously, or may be administered at different times. The dosage of the concomitant drug may be determined according to the dose clinically used, and can be appropriately selected depending on an administration subject, administration route, disease, combination and the like.

In some embodiments of said use or treatment as described in any of the above aspects, the Compound A is administered in combination therapy with levodopa or a levodopa/DDCI preparation.

It has been found that Compound A can be administered simultaneously with levodopa or a levodopa/DDCI preparation; however, a potentially clinically meaningful DDI in PD patients cannot be excluded.

As such, when used in combination, the administration regimen of compound A and levodopa (or a levodopa/DDCI preparation) may differ: each may be administered simultaneously (at the same time) or at different times, for example one before or after the other. A skilled person can readily establish when it may be necessary to administer compound A and levodopa (or a levodopa/DDCI preparation) simultaneously or at different times. For example, should the simultaneous administration of compound A and levodopa (or a levodopa/DDCI preparation) produce undesired effects (for example, dyskinesias and/or dopaminergic side effects such as nausea, vomiting, hallucinations, dizziness or hypotension) due to an undesirable increase in systemic exposure to levodopa, compound A and levodopa (or a levodopa/DDCI preparation) should preferably be administered at different times.

In some embodiments, said use or treatment as described in any of the above aspects comprises administering Compound A, or a pharmaceutically acceptable salt thereof, simultaneously with a daily dose of levodopa or a levodopa/DDCI preparation. For example, Compound A is administered at the same time as a daily administration of levodopa or a levodopa/DDCI preparation, or sequentially within less than 30 minutes, preferably less than 20 minutes, more preferably less than 10 minutes between the administration of compound A and a daily administration of levodopa or of a levodopa/DDCI preparation.

In some embodiments, said use or treatment as described in any of the above aspects comprises administering Compound A, or a pharmaceutically acceptable salt thereof, before or after a daily dose of levodopa or a levodopa/DDCI preparation. In some embodiments, Compound A is administered at least 30-50 minutes, preferably at least one hour, before or after a daily administration of levodopa or of a levodopa/DDCI preparation. In other embodiments compound A is administered from 30 to 150 minutes before or after a daily administration of levodopa (or a levodopa/DDCI preparation).

When compound A is administered after a daily administration of levodopa (or levodopa/DDCI preparation), preferably the subsequent administration of levodopa (or levodopa/DDCI preparation) is administered at least 30 minutes, preferably at least 50 minutes, more preferably at least one hour, after the administration of compound A.

When compound A is administered before a daily administration of levodopa (or levodopa/DDCI preparation), preferably the previous administration of levodopa (or levodopa/DDCI preparation) is administered at least 30 minutes, preferably at least 50 minutes, more preferably at least one hour, before the administration of compound A.

In some embodiments, said use or treatment as described in any of the above aspects comprises administering Compound A, or a pharmaceutically acceptable salt thereof, in the morning, afternoon, evening, prior to sleep, before bedtime or at bedtime.

Still in other embodiments, said use or treatment as described in any of the above aspects comprises administering Compound A, or a pharmaceutically acceptable salt thereof, in the morning, afternoon, evening, before or after a daily dose of levodopa or levodopa/DDCI preparation.

In other embodiments, said use or treatment as described in any of the above aspects comprises administering Compound A, or a pharmaceutically acceptable salt thereof, prior to sleep, before bedtime or at bedtime.

Still in other embodiments, said use or treatment as described in any of the above aspects comprises administering Compound A, or a pharmaceutically acceptable salt thereof, prior to sleep, before bedtime or at bedtime, before or after the last daily dose of levodopa or levodopa/DDCI preparation has been given to the patient and before the following day's dosage of levodopa or levodopa/DDCI preparation is administered.

The term ‘prior to sleep’ means that compound A is administered shortly before the patient goes to sleep, for example less than 90 minutes prior to sleep, particularly less than one hour prior to sleep, less than 30 minutes prior to sleep or immediately prior to sleep.

The term ‘before bedtime’ (i.e. before going to bed) means particularly less than 90 minutes before going to bed, particularly less than 60 minutes before going to bed or less than 30 minutes before going to bed. The term ‘at bedtime’ means less than 5 minutes before bedtime, for example on going to bed.

In other words, compound A is taken by the patient before the patient goes to bed (i.e. before bedtime or at bedtime), e.g. less than 90 minutes before bedtime, particularly less than 60 minutes before bedtime, less than 30 minutes before bedtime or less than 5 minutes before bedtime.

As will be clear in the context of the invention, the term ‘prior to sleep’ or ‘before bedtime’ does not mean any time in the day prior to sleep or going to bed, and in particular does not include, for example, 12 hours before sleep or going to bed. Rather this term means the drug is taken in the period close to the patient going to sleep and probably as part of the patient's bedtime routine.

The administration mode of the concomitant drug is not particularly limited, and Compound A and the concomitant drug only need to be combined as a result of administration. Examples of such administration mode include the following:
(1) administration of a single preparation obtained by simultaneously processing the compound of the present invention and the concomitant drug, (2) simultaneous administration of two kinds of preparations of the compound of the present invention and the concomitant drug, which have been separately produced, by the same administration route, (3) administration of two kinds of preparations of the compound of the present invention and the concomitant drug, which have been separately produced, by the same administration route in a staggered manner, (4) simultaneous administration of two kinds of preparations of the compound of the present invention and the concomitant drug, which have been separately produced, by different administration routes, (5) administration of two kinds of preparations of the compound of the present invention and the concomitant drug, which have been separately produced, by different administration routes in a staggered manner (e.g., administration in the order of the compound of the present invention and the concomitant drug, or in the reverse order) and the like.

The dose of the concomitant drug can be appropriately determined based on the dose employed in clinical situations. The mixing ratio of the compound of the present invention and a concomitant drug can be appropriately determined depending on the administration subject, administration route, target disease, symptom, combination and the like.

Compound A can be used as it is or in the form of a pharmaceutical composition (also referred to as a medicament) by mixing with a pharmacologically acceptable carrier etc. As pharmacologically acceptable carriers, various organic or inorganic carrier substances conventionally used as preparation materials can be used. These are incorporated as excipient, lubricant, binder and disintegrant for solid preparations; or solvent, solubilizing agent, suspending agent, isotonicity agent, buffer and soothing agent for liquid preparations; and the like; and preparation additives such as preservative, antioxidant, colorant, sweetening agent and the like can be added as necessary.

Examples of the dosage form of the above-mentioned pharmaceutical composition include oral preparations such as tablet (including sugar-coated tablet, film-coated tablet, sublingual tablet, orally disintegrating tablet, buccal tablet), capsule (including soft capsule, microcapsule), pill, granule, powder, troche, syrup, liquid, emulsion, suspension, aerosol, films (e.g., orally disintegrable films, oral mucosa-adhesive film) and the like; and parenteral agents such as injection (e.g., subcutaneous injection, intravenous injection, intramuscular injection, intraperitoneal injection, drip infusion), external preparation (e.g., transdermal absorption type preparation, ointment, lotion, adhesive preparation), suppository (e.g., rectal suppository, vaginal suppository), pellet, nasal preparation, pulmonary preparation (inhalant), eye drop and the like. The compound and medicament of the present invention can be respectively safely administered orally or parenterally (e.g., intrarectal, intravenous, intraarterial, intramuscular, subcutaneous, intraorgan, intranasal, intradermal, instillation, intracerebral, intravaginal, and intraperitoneal).

These preparations may be a release control preparation (e.g., sustained-release microcapsule), such as an immediate-release preparation, a sustained-release preparation and the like.

The pharmaceutical composition can be produced according to a method conventionally used in the field of pharmaceutical formulation.

The various aspects and embodiments of the invention as described above relate to uses and methods involving Compound A or a pharmaceutically acceptable salt thereof. The skilled person will appreciate that these aspects and embodiments can also be formulated as the use of Compound A or a pharmaceutically acceptable salt thereof in the manufacture of a medicament for use in the described uses, methods and treatments.

### BRIEF DESCRIPTION OF THE DRAWINGS

FIG. 1—GluCer and LacCer Isomer Percent Change From Predose Over Time (All Doses Combined)

FIG. 2—GluCer and LacCer Percent Change from Predose Over Time in Subjects With GBA-PD with Gaucher Mutation Compared to Non-Gaucher Mutation

FIG. 3—Superimposition of amount of Compound A in human brain at three dose ranges of 10 mg, 30 mg and 60 mg on in vitro CGase activity in human brain homogenates by Compound A (ACurve)

### DETAILED DESCRIPTION

The practice of the present invention employs, unless otherwise indicated, conventional techniques of formulation chemistry, organic chemistry, pharmacology, cell biology, and biochemistry.

Various aspects of the invention are set forth below in sections; however, aspects of the invention described in one particular section are not to be limited to any particular section.

### Definitions

To facilitate an understanding of the present invention, a number of terms and phrases are defined below.

The terms “a” and “an” as used herein mean “one or more” and include the plural unless the context is inappropriate.

As used herein, the term “solid dosage form” refers to the combination of an active agent with at least one carrier or excipient, inert or active, making the composition especially suitable for diagnostic or therapeutic use in vivo or ex vivo.

As used herein “treating” or “treatment” of a disease includes: (1) inhibiting the disease, i.e. arresting or reducing the development of the disease or its clinical symptoms; and/or (2) relieving the disease, i.e. causing regression of the disease or its clinical symptoms.

As used herein “treating” or “treatment” of Parkinson's disease includes:
(1) Preventing, limiting or delaying clinical motor progression;
(2) Preventing, limiting or delaying decline in motor impairment;
(3) Preventing or delaying the onset and/or development of Parkinson's disease;
(4) Preventing, limiting or delaying a decline in cognitive impairment;
(5) Preventing, limiting or delaying a decline in quality of life;
(6) Improving gait speed;
(7) Preventing, limiting or delaying decline in gait speed;
(8) Preventing, limiting or delaying non-motor symptoms progression;
(9) Postponement of late motor complications;
(10) Limiting or delaying of disability caused by Parkinson's disease;
(11) Delaying or slowing of disease progression; prevention;
(12) Having an effect on or change the disease course; and/or
(13) Preventing, limiting and/or delaying rate of disease progression;
in a subject suffering from Parkinson's disease (when compared to a subject administered a placebo or not administered compound A, i.e., not treated).

As used herein the term “clinical motor progression” refers to, but not limited to, worsening of cardinal motor features as rigidity, bradykinesia and tremor.

The term “suffering” or “suffers” as it relates to the term “treatment” refers to a patient or individual who has been diagnosed with or is predisposed to the disease. A patient may also be referred to as being “at risk of” Parkinson's Disease because of a history of disease in their family lineage or because of the presence of genetic mutations associated with the disease.

5,7-dimethyl-N-((1S*,4S)-4-(pentyloxy)cyclohexyl)pyrazolo[1,5-a]pyrimidine-3-carboxamide (Compound A, also known as LTI-291 or BIA 28-6156) has the following structure:

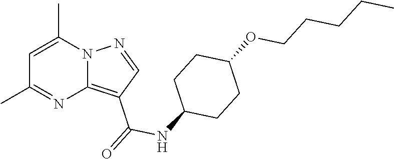 [SMILES: CCCCCO[C@H]1CC[C@H](NC(=O)c2cnn3c(C)cc(C)nc23)CC1]

Compound A is present in four polymorphic forms: Form A, Form B (thermodynamically most stable form at room temperature), Form C (metastable form) and Form D (hydrated form), which can be prepared using the methods described in WO2019/126776.

PD can be assessed using the following scores and measures. The MDS-UPDRS (Goetz, 2008), the Parkinson's Disease Cognitive Rating Scale (PD-CRS) (Pagonabarraga, 2008), the modified Hoehn and Yahr scale (Goetz, 2004), and the 39-Item Parkinson's Disease Questionnaire (PQD-39) (Peto, 1998), are scales that have been specifically developed to follow the longitudinal course of PD and response to treatment. These scales are reliably measurable; meaningful to clinicians, patients, and caregivers; and address the “core” symptoms of PD. The EQ-5D-5L (EuroQol 5 Dimension 5 Level) scale (Herdman, 2011) is a widely used patient-rated survey instrument for measuring economic preferences for health states, is applicable to a wide variety of health conditions and treatments, and provides a simple descriptive profile and a single index value for health status.

The Modified Hoehn and Yahr score is used to assess the staging of the functional disability associated with Parkinson's disease. It helps in describing the progression of the disease through various stages, thus allowing the measurement of the severity of a case. The clinical signs are set out below:

| Stage | Modified Hoehn and Yahr Scale |
|---|---|
| 1 | Unilateral involvement only |
| 1.5 | Unilateral and axial involvement |
| 2 | Bilateral involvement without impairment of balance |
| 2.5 | Mild bilateral disease with recovery on pull test |
| 3 | Mild to moderate bilateral disease; some postural instability; |
| | physically independent |
| 4 | Severe disability; still able to walk or stand unassisted |
| 5 | Wheelchair bound or bedridden unless aided |

The MDS-UPDRS (Goetz, 2008) is a multidimensional scale that assesses the motor and nonmotor impacts of PD across 4 parts. The scale is completed using a combination of physician and patient assessments and a collection of information from the patient or caregiver:
Part I, nonmotor aspects of experiences of daily living, comprises 13 items, 6 of which are rated by the physician (Part IA) and 7 of which are rated by the patient (Part IB).
Part II, motor aspects of experiences of daily living, comprises 13 items that are rated by the patient and/or caregiver. The 13 items in Part II and the 7 items in Part IB constitute the patient questionnaire portion of the MDS-UPDRS.
Part III, motor examination, comprises 18 items that are assessed by the investigator (resulting in 33 scores by location and lateralization).
Part IV, motor complications, comprises 6 items (2 items for dyskinesia, 3 items for fluctuation and 1 for “OFF” dystonia) and requires the physician to use historical and objective information to assess dyskinesia and motor fluctuations.

Each item is rated on a scale from 0 to 4 on which 0=normal, 1=slight, 2=mild, 3=moderate, and 4=severe.

The PD-CRS (Pagonabarraga, 2008) is a short PD-specific questionnaire that is designed to cover the full spectrum of cognitive defects that are associated with PD. It includes items to assess fronto-subcortical defects and items to assess cortical dysfunction. Tasks are included to assess immediate free recall verbal memory (score, 0-12), confrontation naming (score, 0-20), sustained attention (score, 0-10), working memory (score, 0-10), unprompted drawing of a clock (score, 0-10), copy drawing of a clock (score, 0-10), delayed free recall verbal memory (score, 0-12), alternating verbal fluency (score, 0-20), and action verbal fluency (score, 0-30). The score for each task is based on the number of correct responses. The subcortical (range, 0-114) and cortical (range, 0-20) PD-CRS scores are obtained by adding the raw scores of the items within each group. The total score on the PD-CRS are calculated by adding the subcortical and cortical PD-CRS scores.

The CGI-C is a 7-point scale that requires the clinician to assess how much the patient's illness has improved or worsened relative to the baseline state at the beginning of the intervention (Guy, 1976). The raters select one response based on the following question, “Compared to your patient's condition at the beginning of treatment, how much has your patient changed?” Scores are as follows: 1=very much improved; 2=much improved; 3=minimally improved; 4=no change; 5=minimally worse; 6=much worse; and 7=very much worse.

The CGI-S is a 7-point scale that requires the clinician to rate the severity of the patient's illness at the time of assessment, relative to the clinician's past experience with patients who have the same diagnosis (Guy, 1976). Clinicians ask: “Considering your total clinical experience with this particular population, how ill is the patient at this time?” Possible ratings are: 1) Normal, not at all ill, 2) Borderline ill, 3) Mildly ill, 4) Moderately ill, 5) Markedly ill, 6) Severely ill, and 7) Among the most extremely ill patients.

The PGI-S is the patient-reported counterpoint to the CGI-S (Guy, 1976). The PGI-S is a 1-item questionnaire that is designed to assess the patient's impression of disease severity. The PGI-S item asks the respondents to best describe how their symptoms are now on the following 4-point scale: 1=normal, 2=mild, 3=moderate, or 4=severe.

The PGI-C is the patient-reported outcome counterpoint to the CGI-C. The qualitative assessment of meaningful change is determined by the patient's perception of their condition in response to the question, “Compared to your condition at the beginning of treatment, how much has your condition changed?” Scores are as follows: 1=very much improved; 2=much improved; 3=minimally improved; 4=no change; 5=minimally worse; 6=much worse; and 7=very much worse.

The PDQ-39 is the most thoroughly validated and extensively used self-report measure for the assessment of health-related quality of life in patients with PD. The questionnaire measures 39 items, which assess 8 domains of health: mobility (10 items), activities of daily living (6 items), emotional well-being (6 items), stigma (4 items), social support (3 items), cognitions (4 items), communication (3 items), and bodily discomfort (3 items) (Peto, 1998). Each item is scored on the following scale: 0=never, 1=occasionally, 2=sometimes, 3=often, and 4=always. Items in each subscale and the total scale can be summarized into an index and transformed linearly to a scale from 0 (perfect health as assessed by the measure) to 100 (worst health as assessed by the measure).

The EQ-5D-5L (Herdman, 2011) is a patient-reported outcome that measures health in 5 dimensions. It is a widely used survey instrument for measuring economic preferences for health states, is applicable to a wide variety of health conditions and treatments, and provides a simple descriptive profile and a single index value for health status. The EQ-5D-5L consists of a descriptive system and a visual analog scale (VAS).

The descriptive system comprises 5 dimensions: mobility, self-care, usual activities, pain/discomfort, and anxiety/depression. Each dimension has 5 levels: no problems, slight problems, moderate problems, severe problems, and extreme problems. Patients are asked to indicate their health state by ticking the box next to the most appropriate statement in each of the 5 dimensions. This decision results in a 1-digit number that expresses the level that was selected for that dimension. The digits for the 5 dimensions are combined into a 5-digit number that describes the patient's health state.

The EQ-5D-5L VAS records the patient's self-rated health on a vertical VAS on which the endpoints are labeled “the best health quality that you can imagine” and “the worst health quality that you can imagine.” The VAS can be used as a quantitative measure of health outcome that reflects the patient's own judgment.

The Montreal Cognitive Assessment (MoCA) is a widely used screening assessment for detecting cognitive impairment. It was validated in the setting of mild cognitive impairment (MCI), and has subsequently been adopted in numerous other clinical settings. This test consists of 30 points and includes an assessment of short-term memory, executive function, attention, and focus. MoCA scores range between 0 and 30.

It should be understood that elements and/or features of the various aspects, methods, uses, etc. described herein can be combined in a variety of ways without departing from the spirit and scope of the present invention, whether explicit or implicit herein. For example, where reference is made to particular populations of subjects (optionally defined by genetic characteristics), testing criteria or treatment options, such features are applicable to and can be used in various embodiments of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein, where appropriate.

### EXAMPLES

The following examples are provided to illustrate various embodiments of the invention, and are not to be considered limiting. “LTI-291” and “BIA-28” are used in the study numbers to indicate studies with Compound A.

### Example 1—Clinical Studies in Humans

Compound A has been evaluated in two Phase 1 studies to assess the safety, tolerability, PK, and pharmacodynamic effects of single and multiple doses of Compound A in healthy volunteers and in two Phase 1b studies to assess the safety, tolerability, PK, and pharmacodynamic effects of Compound A in subjects with Parkinson's disease with a mutation in the GBA1 gene (GBA-PD). In addition, Compound A has been evaluated in a Phase 1 mass balance recovery study, a Phase 1 thorough QTc study, and a Phase 1 drug-drug interaction study. The design features of these studies are summarized in Table 2 below.

TABLE 2
Overview of Clinical Studies

| | | | | Total Number of |
|---|---|---|---|---|
| | | | | Subjects and |
| | | | | Age Range |
| Study | | | | (Number Who |
| Number | | | | Received |
| (Status) | Study Design | Key Objectives | Treatments/Doses | Compound A) |
| LTI-291-001 | Phase 1, randomized, phase 1, | Safety, tolerability, | Placebo, 3, 10, 30, 90 mg | 40 subjects aged |
| | double-blind, placebo-controlled, | pharmacokinetics (PK), | Compound A, single oral | 18 to 64 years |
| | single, oral, dose-escalation | and pharmacodynamics of | dose in fasted state. | (32 subjects) |
| | study in healthy subjects. | single oral doses of | 10 mg in fed state | |
| | Crossover, food effect | Compound A in healthy | | |
| | Pharmacodynamic assessments: | subjects. | | |
| | GluCer changes in plasma and | Effect of food on | | |
| | PBMCs; CNS tests | Compound A PK | | |
| LTI-291-002 | Phase 1, randomized | Safety, tolerability, PK, | Placebo, 3, 10, 30, 60 mg | 39 subjects aged |
| | double-blind, placebo-controlled, | and pharmacodynamics of | Compound A, multiple oral | 52 to 75 years |
| | multiple, oral, dose-escalation | multiple oral doses of | doses (once daily for 14 | (31 subjects) |
| | study in healthy subjects. | Compound A in healthy | days) | |
| | Pharmacodynamic assessments: | middle-aged to elderly | | |
| | GluCer, GluSph, and LacCer in | subjects. | | |
| | plasma, PBMCs, and CSF; CNS | | | |
| | tests | | | |
| | Compound A concentrations in | | | |
| | plasma and CSF | | | |
| LTI-291-003 | Phase 1b, randomized, | Safety, tolerability, PK, and | Placebo, 10, 30, 60 mg | 40 subjects aged |
| | double-blind, | pharmacodynamics of | Compound A, | 40 to 80 years |
| | placebo-controlled, | multiple oral doses of | multiple oral doses (once | (30 subjects) |
| | parallel, multiple oral dose | Compound A in subjects | daily for 28 days) | |
| | study in patients. | with Parkinson's disease | | |
| | Pharmacodynamic | with a mutation in the | | |
| | assessments: GluCer | GBA1 gene (GBA-PD). | | |
| | changes in plasma and | | | |
| | PBMCs; CNS tests | | | |
| LTI-291-004 | Phase 1b, randomized, | Safety, tolerability, PK, and | Placebo, 10, 60 mg | 14 subjects aged |
| | double-blind, | pharmacodynamics by | Compound A, | 51 to 81 years |
| | placebo-controlled, | fMRI and FDG-PET of | multiple oral doses (once | (12 subjects) |
| | parallel, multiple oral dose | multiple oral doses of | daily for 28 days) | |
| | study in patients | Compound A in subjects | | |
| | Pharmacodynamic | with GBA-PD. | | |
| | assessments: GluCer, | | | |
| | GluSph, and LacCer in | | | |
| | plasma and PBMCs | | | |
| BIA 28-6156-105 | Phase 1, randomized, | QTcF, safety, tolerability, | Placebo, 60 mg Compound A, | 37 subjects aged |
| | double-blind, placebo- and | and PK of single oral doses | 150 mg Compound A, and | 20 to 55 years |
| | positive-controlled, 4-way | of Compound A 60 mg | 400 mg moxifloxacin single | (36 subjects) |
| | crossover, thorough QTc | (therapeutic dose) and 150 | oral doses | |
| | study in healthy male and | mg (supratherapeutic | | |
| | female subjects under fed | dose) | | |
| | conditions | | | |
| BIA 28-6156-106 | Phase 1, single-dose, | Absorption, distribution, | 14C-Compound A 60 mg, | 6 subjects aged |
| | single-period, open label, | metabolism, and | containing not more than 3.7 | 32 to 55 years |
| | mass balance recovery, | elimination (ADME) in | MBq 14C | (6 subjects) |
| | metabolite profile, and | humans; PK; safety; | Actual mean dose was 60.12 | |
| | metabolite identification of | metabolite profiling and | mg and 14C-Compound A | |
| | 14C-Compound A in | structural identification | exposure of 2.91 MBq | |
| | healthy male subjects | | | |
| BIA 28-6156-107 | Phase 1, non-randomized, | PK of Compound A with | 60 mg Compound A on Days | 12 subjects aged |
| | open-label, crossover, | and without | 2 and 25; multiple dose | 24 to 69 years |
| | drug-drug interaction | carbamazepine; Safety and | carbamazepine ER (titrated | (12 subjects) |
| | study of the effects of | tolerability of Compound A | to 300 mg BID) on Days 9 | |
| | carbamazepine on single | | through 31 | |
| | dose Compound A in | | | |
| | heathy male and female | | | |
| | subjects | | | |
| BIA 28-6156-107 | Phase 1, non-randomized, | PK of carbidopa, levodopa, | carbidopa-levodopa IR on | 12 subjects aged |
| | open-label, crossover, DDI | and 3-OMD with and | Days 1 and 8; 60 mg | 24 to 69 years |
| | study of the effects of | without BIA 28-6156; | Compound A on Day 8 | (12 subjects) |
| | Compound A on single | Safety and tolerability of | | |
| | dose carbidopa-levodopa | Compound A | | |
| | in healthy male and female | | | |
| | subjects | | | |
| BIA 28-6156-109 | Phase 1, non- | To investigate the effects | Cohort 1 (n = 12): | 24 subjects aged |
| | randomized, open- | of multiple doses of | On Day 1: a single dose of 100 mg/25 | 18 to 75 years |
| | label, 2-cohort, | Compound A on the | mg levodopa/carbidopa (Sinemet ®) | |
| | cross-over, drug- | single-dose | immediate release (IR) was administered | |
| | drug interaction | pharmacokinetics (PK) of | under fed conditions. | |
| | (DDI) study to | Sinemet ® (100 mg/25 | From Day 2 to Day 6: a single dose of 60 | |
| | evaluate the | mg levodopa/carbidopa) | mg Compound A was administered under | |
| | effects of multiple | in healthy male and | fed conditions. | |
| | doses of | female subjects. | On Day 7: a single dose of 60 mg | |
| | Compound A | To investigate the effects | Compound A and a single dose of 100 | |
| | on the single-dose | of multiple doses of | mg/25 mg levodopa/carbidopa (Sinemet ®) | |
| | PK and safety of | Compound A on the | IR were administered under fed conditions. | |
| | levodopa- | single-dose PK of | Cohort 2 (n = 12): | |
| | carbidopa | Madopar ® (100 mg/25 | On Day 1: a single dose of 100 mg/25 | |
| | and of levodopa- | mg | mg levodopa/benserazide (Madopar ®) IR | |
| | benserazide, | levodopa/benserazide) | was administered under fed conditions. | |
| | respectively, in | in healthy male and | From Day 2 to Day 6: a single dose of 60 | |
| | healthy male and | female subjects. | mg of Compound A was administered | |
| | female subjects | | under fed conditions. | |
| | | | On Day 7: a single dose of 60 mg of | |
| | | | Compound A and a single dose of 100 | |
| | | | mg/25 mg levodopa/benserazide | |
| | | | (Madopar ®) IR was administered under fed | |
| | | | conditions. | |
| BIA 28-6156-110 | Phase 1, non- | To investigate the effects | The following treatments were | A total of 32 |
| | randomized, open- | of Compound A on the | administered: | healthy male |
| | label, 2-cohort, | single-dose | Cohort 1 (n = 16) | and female |
| | cross-over, drug- | pharmacokinetics | Day 1: a single dose of 10 mg | subjects were |
| | drug interaction | (PK) of rosuvastatin in | rosuvastatin was administered under fed | enrolled. Female |
| | (DDI) study to | healthy male and female | conditions | subjects were of |
| | evaluate the | subjects. To investigate | Day 2: no drug was administered | non-childbearing |
| | effects of | the effects of Compound | Day 3 to Day 8: a single daily dose of 60 | potential or |
| | Compound A on | A on the single-dose PK | mg Compound A was administered under | postmenopausal. |
| | the single-dose PK | of metformin. To assess | fed conditions | |
| | and the safety and | the safety and tolerability | Day 9: a single dose of 60 Compound A | |
| | tolerability of | of co-administration of | and a single dose of 10 mg of rosuvastatin | |
| | rosuvastatin and | Compound A with | were administered | |
| | metformin in | Rosuvastatin. To assess | under fed conditions | |
| | healthy male and | the safety and tolerability | Cohort 2 (n = 16) | |
| | female subjects. | of co-administration of | Day 1: a single dose of 500 mg | |
| | | Compound A with | metformin was administered under fed | |
| | | Metformin. | conditions | |
| | | | Day 2 to Day 7: a single daily dose of 60 | |
| | | | mg Compound A was administered under | |
| | | | fed conditions | |
| | | | Day 8: a single dose of 60 mg Compound | |
| | | | A and a single dose of 500 mg of | |
| | | | metformin were administered | |
| | | | under fed conditions | |

Abbreviations: CNS = central nervous system; CSF = cerebrospinal fluid; FDG-PET = fluorodeoxyglucose-positron emission tomography; fMRI = functional magnetic resonance imaging; GBA1 = human gene for glucosidase, beta, acid, aka glucocerebrosidase (GCase); GBA-PD = Parkinson's disease with a mutation in the GBA1 gene; GluCer = glucosylceramide; GluSph = glucosylsphingosine; LacCer = lactosyl-ceramide; PBMC = peripheral blood mononuclear cell; PK = pharmacokinetics.

In the Phase 1b studies, 40 subjects (20 men and 20 women) with GBA-PD, aged 40 to 80 years, received multiple doses of 10, 30, or 60 mg of Compound A or placebo once daily for up to 28 days (Study LTI-291-003) and 14 subjects (13 men and 1 woman) received multiple doses of 10 or 60 mg of Compound A or placebo once daily for up to 28 days (Study LTI-291-004).

The PK of Compound A was similar between healthy subjects and subjects with GBA-PD. Geometric mean CSF concentrations increased with increasing dose, and the CSF-to-plasma concentration ratios were similar between dose levels after the 28th daily dose. Mean values for the ratios corresponded with the approximate 1:1 distribution of unbound Compound A in plasma to CSF, indicating that Compound A is found in CSF and can cross the blood brain barrier to reach GCase in brain tissue.

### LTI-291-001—Single, Ascending-Dose Study

This study was a first-in-human Phase 1, randomized, double-blind, placebo-controlled, single-ascending dose study to evaluate the safety and tolerability, PK, and PD of escalating single oral doses of Compound A (3, 10, 30, and 90 mg) in 4 cohorts of healthy subjects. A total of 32 subjects received a single oral dose of Compound A (8 per dose group) and 8 subjects received placebo. The effect of food on the PK of Compound A was evaluated by administering a second single oral dose of 10 mg Compound A to the subjects in Cohort 2 on a separate occasion, after subjects had completed a high-fat breakfast. Blood samples for Compound A PK were collected up to 48 hours after each dose.

The pharmacodynamic evaluations consisted of a battery of CNS (NeuroCart®) assessments, including Saccadic Eye Movements, Smooth Pursuit Eye Movements, Adaptive Tracking, Body Sway, Visual Verbal Learning Test, and EEG. GluCer was measured in plasma and PBMCs as a biomarker of pharmacological effect.

### Pharmacokinetic Results

The median Tₘₐₓ occurred within 1 to 3 hours (median) and ranged between 1.0 and 8.2 after a single dose under fasted conditions. No relationship was observed between Tₘₐₓ and dose level. Geometric mean t_{1/2} of Compound A was similar among fasted and fed subjects, ranging from 21.2 to 23.4 hours across groups. Across the entire dose range, total exposure (Cₘₐₓ and AUC_{0-inf}) appeared to increase in a dose proportional manner, with similar values for dose normalized AUC and Cₘₐₓ across dose levels. Dosing 10 mg in the fed state led to a later Tₘₐₓ (median of 8.0 hours versus 1.0 hour under fasted conditions), a lower Cₘₐₓ, but similar t_{1/2} and AUC compared to dosing in the fasted state.

A summary of the PK parameters of LTI-291 following single oral administration to healthy participants in the fed and fasted state is presented in Table 3 below:

TABLE 3
Study LTI-291-001: Summary of Plasma Compound A Pharmacokinetic Parameters
in Health Subjects Following Single Oral Doses of Compound A

| Compound A Dose | | | | |
|---|---|---|---|---|
| 3 mg Fasted | 10 mg Fasted | 10 mg Fed | 30 mg Fasted | 90 mg Fasted |

| | | | |
|---|---|---|---|
| N = 8 | N = 8 (Crossover) | N = 8 | N = 8 |

Geometric mean (% CV geometric mean)

| | | | | | | | | | | |
|---|---|---|---|---|---|---|---|---|---|---|
| Cₘₐₓ (ng/mL) | 109 | (12.0) | 328 | (39.9) | 234 | (29.8) | 961 | (30.3) | 2640 | (31.3) |
| t_{1/2} (h) | 21.2 | (54.1) | 22.2 | (39.9) | 23.4 | (47.4) | 23.2 | (34.3) | 23.3 | (23.7) |
| AUC₀₋ₗₐₛₜ | 1560 | (24.4) | 5690 | (16.4) | 5860 | (19.2) | 18400 | (20.8) | 47800 | (17.6) |
| (ng · h/mL) | | | | | | | | | | |
| AUC_{0-inf} | 2260 | (30.3) | 7560 | (27.8) | 8530 | (30.8) | 24600 | (12.3) | 63400 | (15.5) |
| (ng · h/mL) | | | | | | | | | | |
| CL (L/h) | 1.33 | (30.3) | 1.32 | (27.8) | 1.17 | (30.8) | 1.22 | (12.3) | 1.42 | (15.5) |
| V_{z}/F (L) | 40.5 | (34.2) | 42.4 | (24.9) | 39.6 | (27.2) | 40.8 | (40.5) | 47.7 | (26.0) |
| Cₘₐₓ/dose | 36.4 | (12.0) | 32.8 | (39.9) | 23.4 | (29.8) | 32.0 | (30.3) | 29.4 | (31.3) |
| (ng/mL / mg) | | | | | | | | | | |
| AUC_{0-inf}/dose | 754 | (30.3) | 756 | (27.8) | 853 | (30.8) | 821 | (12.3) | 704 | (15.5) |
| (ng · h/mL / mg) | | | | | | | | | | |

Median (minimum, maximum)

| | | | | | |
|---|---|---|---|---|---|
| Tₘₐₓ (h) | 1.48 | 1.01 | 8.00 | 3.02 | 2.00 |
| | (1.00, 2.00) | (1.00, 4.02) | (4.00, 12.0) | (1.00, 4.00) | (1.00, 8.17) |

Abbreviations: AUC_{0-inf} = area under the plasma concentration versus time curve (AUC) extrapolated to infinity; AUC₀₋ₗₐₛₜ = AUC from time zero to the time of the last measurable concentration; CL/F = apparent total body clearance; Cₘₐₓ = maximum observed plasma drug concentration; t_{1/2} = terminal elimination half-life; Tₘₐₓ = time of maximum observed plasma drug concentration; V_{z}/F = apparent volume of distribution during the terminal phase.

The ratio (90% CI) of geometric means for the fed to fasting treatment comparison was 71.3% (54.2 to 93.6%) for Cₘₐₓ. The ratios for AUC₀₋ₗₐₛₜ and AUC_{0-inf} were 103% and 111%, and 90% CIs were entirely contained within 80.0 to 125.0%.

### Pharmacodynamic Results

There were no dose-dependent effects of Compound A on any of the CNS (Neurocart) tests. No significant overall treatment effects were observed on the GluCer variables tested in plasma. Differences between treatment with Compound A and placebo were observed in some GluCer variables measured in PBMCs, indicating increased glycolipid flux with Compound A treatment.

### Safety Results

No deaths, other SAEs, severe AEs, or discontinuations due to AEs were reported. The most common AEs following Compound A were fatigue (3 subjects, 7.5%), headache (5 subjects, 12.5%) and somnolence (4 subjects, 10%), whereas the most common AE following placebo was somnolence (2 subjects, 25%). Three subjects (7.5%) across the Compound A treatment groups reported at least one AE in the GI system organ class, whereas none of the subjects in the placebo group reported any GI AEs. Most AEs were mild in intensity, with only 1 moderate AE (unrelated to Compound A) reported.

There were no clinically significant safety observations from vital sign measurements, laboratory safety assessments, or ECG parameters.

### Multiple-, Ascending-Dose Study

This study was a phase 1, randomized, double-blind, placebo-controlled, multiple ascending dose study of Compound A in 4 cohorts of middle-aged to elderly healthy participants (aged 52 to 75 years). The objectives of the study were to investigate the safety, tolerability, and PK and explore the pharmacodynamics of multiple escalating doses of Compound A that were shown to be well tolerated in the single ascending dose study (LTI-291-001). A total of 39 healthy subjects were enrolled in 4 sequential cohorts. Subjects in each cohort received once-daily oral doses of 3 mg (n=7), 10 mg (n=8), 30 mg (n=8), or 60 mg (n=8) Compound A or placebo (n=8) for 14 days. Pharmacokinetic parameters were determined for each dose at Day 1, Day 7, and Day 14. Cerebrospinal fluid samples for Compound A concentrations were measured before the first dose and 4 hours after the last dose for each dose level. GluCer, glucosyl sphingosine (GluSph), and lactosyl-ceramide (LacCer) were measured in plasma, PBMCs, and CSF as biomarkers of pharmacological effects. Pharmacodynamic measurements consisted of battery of CNS (NeuroCart®) assessments including Saccadic Eye Movements, Smooth Pursuit Eye Movements, Adaptive Tracking, Body Sway, Visual Verbal Learning Test, and pharmaco-EEG.

### Pharmacokinetic Results

Following 1, 7, and 14 consecutive daily oral doses of 3, 10, 30, and 60 mg Compound A, peak plasma concentrations occurred within 1 to 4 hours (median) and ranged between 1.0 to 8.12 hours after dosing. No relationship was observed between the values for Tₘₐₓ and dose level or dose number. Values for mean t_{1/2} ranged from 20.7 to 27.9 hours after the 1st dose, 25.7 to 35.8 hours after the 7th dose, and 32.1 to 75.0 hours after the 14th dose. No trend was observed for t_{1/2} to change as a function of increasing dose. The accumulation results suggested that steady-state conditions were achieved by the 7th daily dose, with values for the accumulation index ranging from 2.11 to 2.72 after the 7th daily dose and 2.49 to 2.78 after the 14th daily dose, excepting the 30 mg dose level (accumulation index=5.22). No trend was observed for the accumulation index to change as a function of increasing dose or dose number. Across the dose range of 3 to 60 mg, total exposure (Cₘₐₓ and AUC₀₋₂₄) appeared to increase in a dose proportional manner after each of the 1st, 7th, and 14th daily oral doses, with similar values for dose normalized AUC and Cₘₐₓ across dose levels.

Geometric mean CSF concentrations increased with increasing dose. The CSF-to-plasma concentration ratios were similar between dose levels after the 14th daily dose, with mean values ranging between 0.0122 to 0.0128. These values correspond with an approximate 1:1 distribution of unbound Compound A in plasma to CSF and indicate Compound A brain penetration at all doses.

A summary of the PK parameters of LTI-291 following single oral administration to healthy participants in the fed and fasted states is presented in Table 4.

TABLE 4
Study LTI-291-002: Compound A Plasma Pharmacokinetic Parameters in Healthy Subjects Following
the First, Seventh, and Fourteenth Daily Oral Dose of Compound A

| | | | | | | | | | | | | |
|---|---|---|---|---|---|---|---|---|---|---|---|---|
| Compound A | 3 mg | 10 mg | 30 mg | 60 mg | 3 mg | 10 mg | 30 mg | 60 mg | 3 mg | 10 mg | 30 mg | 60 mg |
| Dose Level | (n = 7) | (n = 8) | (n = 8) | (n = 8) | (n = 7) | (n = 8) | (n = 8) | (n = 8) | (n = 7) | (n = 8) | (n = 8) | (n = 8) |
| Dose | 1 | 1 | 1 | 1 | 7 | 7 | 7 | 7 | 14 | 14 | 14 | 14 |
| Number | | | | | | | | | | | | |

Geometric Mean (% CV geometric mean)

| | | | | | | | | | | | | |
|---|---|---|---|---|---|---|---|---|---|---|---|---|
| Cₘₐₓ | 89.1 | 307 | 869 | 1950 | 142 | 505 | 1600 | 3190 | 153 | 493 | 1500 | 3070 |
| (ng/mL) | (20.0) | (25.5) | (41.1) | (39.3) | (22.9) | (23.2) | (11.9) | (26.4) | (31.3) | (27.3) | (13.2) | (29.7) |
| t½ | 27.9 | 20.7 | 26.0 | 25.1 | 31.3 | 28.7 | 25.7 | 35.8 | 36.5 | 32.1 | 75.0 | 32.9 |
| (h) | (78.5) | (14.8) | (35.6) | (30.7) | (43.0) | (31.1) | (22.3) | (41.6) | (49.6) | (25.3) | (97.6) | (20.3) |
| AUC₀₋ₗₐₛₜ | 1040 | 3380 | 10400 | 19200 | 2420 | 7820 | 26300 | 50300 | 4510 | 11900 | 45000 | 82000 |
| (ng · h/mL) | (19.4) | (16.9) | (12.8) | (19.4) | (32.3) | (22.5) | (17.5) | (20.6) | (41.7) | (23.0) | (29.6) | (26.7) |
| AUC₀₋₂₄ | 1050 | 3410 | 10600 | 19600 | 1920 | 7730 | 26500 | 52100 | 2770 | 7510 | 26200 | 50900 |
| (ng · h/mL) | (19.5) | (16.8) | (13.6) | (20.9) | (43.1) | (23.6) | (19.0) | (20.5) | (34.7) | (20.9) | (18.6) | (24.6) |
| AUC_{0-inf} | 2450 | 6130 | 22600 | 39300 | 4720 | 17800 | 56800 | 139000 | 7560 | 18600 | 139000 | 130000 |
| (ng · h/mL) | (66.9) | (19.1) | (21.0) | (31.2) | (84.2) | (44.8) | (32.9) | (50.5) | (78.4) | (32.9) | (102) | (35.3) |
| Cₘₐₓ/dose | 29.7 | 30.7 | 29.0 | 32.6 | 47.2 | 50.5 | 53.3 | 53.2 | 51.0 | 49.3 | 50.0 | 51.2 |
| (ng/mL/mg) | (20.0) | (25.5) | (41.1) | (39.3) | (22.9) | (23.2) | (11.9) | (26.4) | (31.3) | (27.3) | (13.2) | (29.7) |
| AUC_{0-inf}/dose | 818 | 613 | 753 | 655 | 1570 | 1780 | 1890 | 2310 | 2520 | 1860 | 4620 | 2160 |
| (ng · h/mL/ | (66.9) | (19.1) | (21.0) | (31.2) | (84.2) | (44.8) | (32.9) | (50.5) | (78.4) | (32.9) | (102) | (35.3) |
| mg) | | | | | | | | | | | | |
| C_{avg} (ng/mL/ | | | | | 80.1 | 322 | 1100 | 2170 | 115 | 313 | 1090 | 2120 |
| mg) | | | | | (43.1) | (23.6) | (19.0) | (20.5) | (34.7) | (20.9) | (18.6) | (24.6) |
| Accumulation | | | | | 2.45 | 2.29 | 2.11 | 2.72 | 2.78 | 2.49 | 5.22 | 2.53 |
| Index | | | | | (31.9) | (23.1) | (14.8) | (33.3) | (38.6) | (19.3) | (77.7) | (15.6) |
| Fluctuation | | | | | | | | | 52.9 | 76.0 | 58.1 | 67.6 |
| (%) | | | | | | | | | (37.4) | (58.6) | (35.5) | (32.0) |

Median (minimum, maximum)

Tₘₐₓ (h)

Abbreviations: AUC_{0-inf} = Area under the plasma concentration versus time curve (AUC) extrapolated to infinity; AUC₀₋ₗₐₛₜ = AUC from time zero to the time of the last measurable concentration; AUC₀₋₂₄ = AUC from time zero to 24 hours; C_{avg} = average plasma drug concentration over the dosing interval; Cₘₐₓ = maximum observed plasma drug concentration; t_{1/2} = terminal elimination half-life; Tₘₐₓ = time of maximum observed plasma drug concentration.

### Pharmacodynamic Results

There were no dose-dependent effects of Compound A on any of the CNS (Neurocart) tests. A significant increase in saccadic inaccuracy was observed in the 10 and 30 mg Compound A groups compared to placebo. No changes in saccadic peak velocity or reaction time were observed. A significant decrease in EEG Alpha-power Pz-Oz in the eyes closed condition (uV) was observed at the 3 mg dose relative to placebo.

Sporadic, statistically significant increases were observed in some GluCer and LacCer variables in plasma and PBMCs, and sporadic, statistically significant decreases were observed in some GluCer and LacCer variables in CSF. Overall, a consistent pharmacodynamic effect of Compound A on glycolipids over 14 days of dosing was not observed in healthy subjects.

### Safety Results

No deaths, other SAEs, severe AEs, or discontinuations due to AEs were reported. All AEs were mild or moderate and self-limited, and no specific pattern of AEs associated with Compound A administration was apparent. The most common AEs following multiple oral doses of Compound A once daily for 14 days were headache (12 subjects, 39%), somnolence (5 subjects, 16%), myalgia (5 subjects, 16%), and back pain (4 subjects, 13%), whereas the most common AEs following placebo were back pain (2 subjects, 25%) and dizziness (2 subjects, 25%).

Somnolence was reported more frequently in the 60 mg dose group (50% of subjects) than in the 3 mg (14%), 10 mg (0%), or 30 mg (0%) dose groups or in the placebo group (13%). In 4 of the 5 subjects who reported somnolence during the study (including the placebo subject), the AE resolved within a couple of hours of onset. For 1 subject, the AE occurred intermittently over a period of 24 days. Somnolence was considered possibly related to Compound A administration, although no effects were seen on NeuroCart measurements.

Myalgia was reported more frequently in the 60 mg dose group (38% of subjects) than in the 3 mg (14%), 10 mg (0%), or 30 mg (13%) dose groups or in the placebo group (13%). The AE started between Day 1 and Day 4 in all subjects and lasted 3 to 10 days in 5 of 6 subjects and resolved within 8 hours in 1 subject (3 mg group). No clinically significant creatine kinase abnormalities were found in the 60 mg group, thereby excluding rhabdomyolysis. There were no clinically significant safety observations from vital sign measurements, laboratory safety assessments (including creatine kinase), or ECG parameters.

### Compound A—Multiple Dose Study in Subjects With PD and a Mutation in the GBA1 Gene (LTI-291-003)

This study was a Phase 1b, randomized, double-blind, placebo-controlled, multiple oral dose study of Compound A in 40 subjects (aged 40 to 80 years) with GBA-PD. Approximately 50% of the enrolled subjects had major (loss of 30%-50% total activity) mutations in GCase and 50% had polymorphism mutation (loss of 10%-20% of total activity) mutations in GCase. The objectives of this study were to investigate the safety, tolerability, and PK of multiple once-daily oral administration of Compound A and to explore the accompanying pharmacodynamic changes in subjects with GBA-PD with major and polymorphism mutations. Subjects received once-daily oral doses of 10 mg (n=10), 30 mg (n=10), or 60 mg (2×30 mg capsules) (n=10) Compound A or placebo (n=10) for 28 consecutive days.

Blood samples for Compound A concentrations were collected up to 6 hours after the first and last doses. CSF samples for Compound A concentrations were taken before the first dose and 4 hours after the last dose for each dose level.

GluCer, GluSph, and LacCer were measured in plasma, PBMCs, and CSF as biomarkers of pharmacological effects. The pharmacodynamic evaluations also consisted of a battery of NeuroCart assessments (Saccadic Eye Movements, Smooth Pursuit Eye Movements, Adaptive Tracking, Body Sway, and Visual Verbal Learning Test); functional outcome measures (MDS-UPDRS Part III, and the MMSE); and pharmaco-EEG.

### Pharmacokinetic Results

Peak plasma concentrations occurred at 2.0 hours (median) after the 1st and 28th daily dose of Compound A and ranged between 2.0 to 6.0 hours across dosing days and dose levels. Because of the limited sampling scheme, t_{1/2} was not estimable. Accumulation of Compound A over the study period was assessed by taking the ratios of the Cₘₐₓ, AUC₀₋ₗₐₛₜ, and AUC₀₋₆ following the 28th dose to the corresponding parameters following the 1st dose. When the data were grouped by dose level, the median ratios for the 3 parameters were between 1.93 and 2.47 and were consistent with results observed in healthy subjects. Across the dose range of 10 to 60 mg, total exposure (Cₘₐₓ and AUC₀₋ₗₐₛₜ) appeared to increase in a dose proportional manner after the 1st and 28th dose, with similar values for dose-normalized AUC and Cₘₐₓ observed across dose levels.

TABLE 5
Study LTI-291-003: Summary of Compound A Plasma
Pharmacokinetic Parameters in Subjects With Parkinson's Disease
With a Mutation in the GBA1 Gene

| Compound A | 10 mg | 10 mg | 30 mg | 30 mg | 60 mg | 60 mg |
|---|---|---|---|---|---|---|
| Dose Level | N = 10 | | N = 10 | | N = 10 | |
| Dose | 1 | 28 | 1 | 28 | 1 | 28 |
| Number | | | | | | |

Geometric mean (% CV geometric mean)

| | | | | | | |
|---|---|---|---|---|---|---|
| Cₘₐₓ (ng/mL) | 254 (34.6) | 520 | 561 (106) | 1440 | 1530 | 2790 |
| | | (39.1) | | (14.5) | (40.4) | (14.2) |
| Cₘᵢₙ (ng/mL) | | 246 | | 788 | | 1370 |
| | | (45.5) | | (21.2) | | (34.7) |
| AUC₀₋ₗₐₛₜ | 1090 | 2520 | 2300 | 7230 | 6100 | 14100 |
| (ng · h/mL) | (32.8) | (38.3) | (101) | (15.1) | (43.9) | (15.7) |
| AUC₀₋₆ | 1080 | 2520 | 2300 | 7230 | 6080 | 14100 |
| (ng · h/mL) | (33.1) | (38.3) | (101) | (15.1) | (43.7) | (15.7) |
| Cₘₐₓ/dose | 25.4 | 52.0 | 18.7 | 47.9 | 25.5 | 46.6 |
| (ng/mL/mg) | (34.6) | (39.1) | (106) | (14.5) | (40.4) | (14.2) |
| AUC₀₋ₗₐₛₜ/dose | 109 (32.8) | 252 | 76.8 | 241 | 102 | 234 (15.7) |
| (ng · h/mL | | (38.3) | (101) | (15.1) | (43.9) | |
| mg) | | | | | | |

Median (minimum, maximum)

| | | | | | | |
|---|---|---|---|---|---|---|
| Tₘₐₓ (h) | 2.00 | 2.00 | 2.00 | 2.00 | 2.00 | 2.00 |
| | (2.00, | (2.00, | (2.00, | (2.00, | (2.00, | (2.00, |
| | 4.05) | 6.00) | 6.02) | 4.00) | 6.00) | 6.00) |
| Tₘᵢₙ (h) | | 26.3 | | 26.1 | | 26.0 |
| | | (23.7, | | (22.5, | | (2.00, |
| | | 29.0) | | 27.8) | | 28.2) |

Abbreviations: AUC₀₋ₗₐₛₜ = area under the plasma concentration versus time curve (AUC) from time zero to the time of the last measurable concentration; AUC₀₋₆ = AUC from time zero to 6 hours; Cₘₐₓ = maximum observed plasma drug concentration; Cₘᵢₙ = minimum observed plasma drug concentration; Tₘₐₓ = time of maximum observed plasma drug concentration; Tₘᵢₙ = time of minimum observed plasma drug concentration.

Geometric mean CSF concentrations increased with increasing dose, and the CSF-to-plasma concentration ratios were similar between dose levels after the 28th daily dose. Mean (SD) values for the ratios were 0.0113 (0.00233) for the 10 mg dose, 0.0122 (0.0036) for the 30 mg dose, and 0.0115 (0.00305) for the 60 mg dose. These results are similar to those observed in the 14-day multiple dose study in healthy subjects (Study LTI-291-002) and correspond with the approximate 1:1 distribution of unbound Compound A in plasma to CSF determined in the preclinical studies.

### Pharmacodynamic Results

There were no dose-dependent effects of Compound A on any of the NeuroCart tests, MMSE, MDS-UPDRS, or EEG.

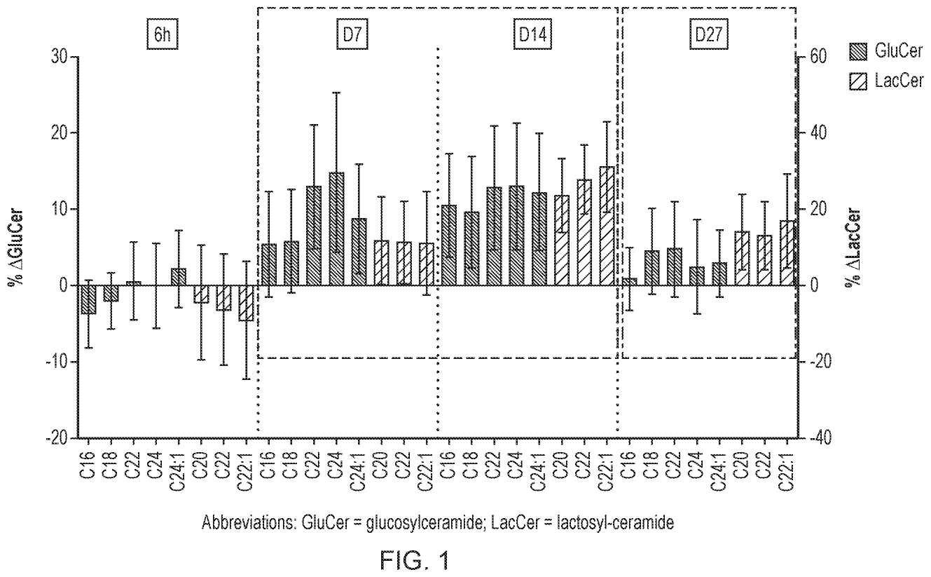

Significant, time-dependent changes in intracellular PBMC glycolipid levels (GluCer and LacCer) were observed in all dose groups (10, 30, and 60 mg/day) of treated subjects with GBA-PD, indicating a change in flux of glycolipids due to Compound A (FIG. 1). This was manifested by a statistically significant increase in GluCer and LacCer in PBMCs at 7 and 14 days of treatment, with a smaller increase observed at 28 days of treatment.

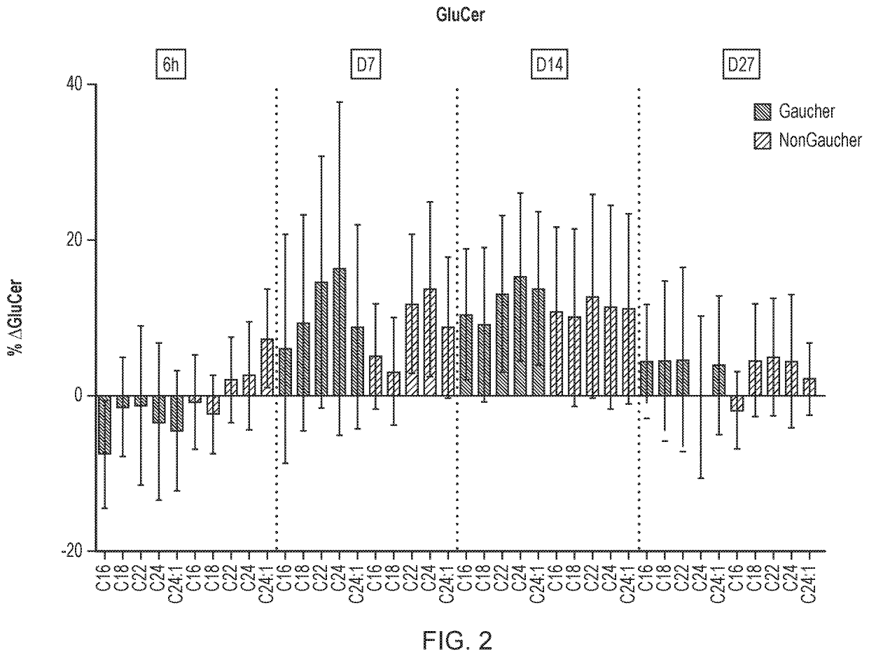

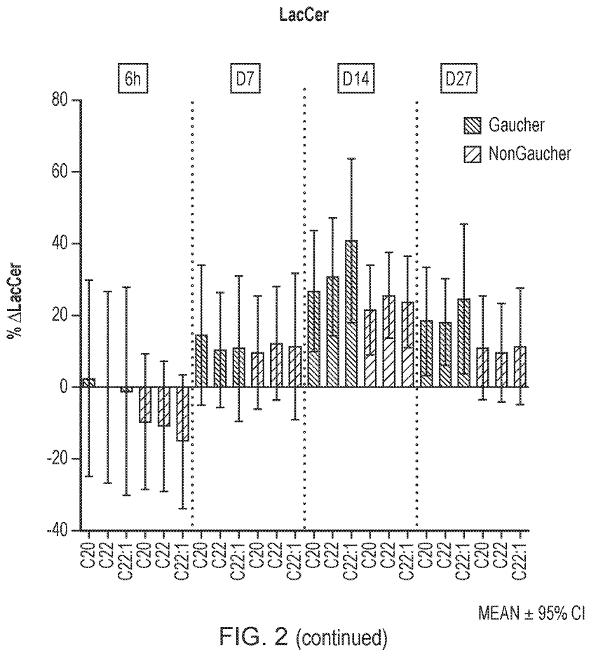

There was no obvious dose-response or exposure response relationship to these effects, although the responder analysis showed the largest changes occurred in the 60 mg GluCer PBMC group (a steep dose-response was not expected, since the exposure at all 3 doses was in excess of what is required to double GCase activity in vitro). The severity of the GBA1 mutation (i.e., extent of loss-of-function and relative risk of PD) was a statistically significant co-variate in the ANCOVA analyses, indicating the effects were generally greater in the subjects with Parkinson's disease with “Gaucher” or major (loss of 30%-50% total activity) mutations versus polymorphism (loss of 10%-20% of total activity) GBA1 mutations (FIG. 2).

This relationship is consistent with the expectation that the GSL pathway in patients with low GCase activity changes more profoundly than the GSL pathway in patients with moderate GCase activity. No GSL pathway changes were observed in healthy elderly subjects. Overall, these changes indicate that Compound A increases GCase activity, increasing GSL ‘flux’ through GCase and changing the levels of key elements of the pathway (GluCer and LacCer).

Intracellular GluSph in PBMCs did not show a clear response to Compound A compared to placebo, and there were no obvious changes in plasma or CSF levels of glycolipids.

### Safety Results

No deaths, other SAEs, severe AEs, or discontinuations due to AEs were reported. The most common AEs following multiple oral doses of Compound A in subjects with GBA-PD were fatigue (5 patients, 16.7%), back pain (4 patients, 13.3%), headache (4 subjects, 13.3%), and worsening of Parkinson's disease symptoms (4 subjects, 13.3%). In the placebo group, the most frequently reported AEs were headache (3 subjects, 30.0%) and fatigue (2 subjects, 20.0%). Worsening of Parkinson's disease symptoms was reported by one subject (10%) in the placebo group. Considering the natural variation in Parkinson's disease symptom severity and the progressive disease course, the worsening of Parkinson's disease symptoms was considered unlikely related to Compound A.

Most AEs were mild in intensity, with only 3 moderate AEs (all unrelated to Compound A) reported; no severe AEs were reported. There were no clinically significant safety observations from vital sign measurements, laboratory safety assessments, or ECG parameters.

### Multiple-Dose Imaging Study in Subjects With Parkinson's Disease and a Mutation in the GBA1 Gene (GBA-PD) (LTI-291-004)

This study was a Phase 1B, randomized, double-blind, placebo-controlled, multiple oral dose study of Compound A in 14 subjects (aged 51 to 81 years) with GBA-PD. The objectives of this study were to investigate the safety, tolerability, and PK of multiple once-daily oral administration of Compound A and to explore the accompanying pharmacodynamic changes. Subjects received once-daily oral doses of 10 mg (n=6) or 60 mg (2×30 mg capsules) (n=6) Compound A or placebo (n=2) for 28 consecutive days. PK samples were measured once on Day 7 and Day 14 and 4 times after the last dose on Day 28. Plasma concentrations were summarized at each time point, and no PK parameters were estimated. The PD evaluations included fMRI measures; FDG-PET to determine the metabolic rate of glucose in specific regions of the brain; and functional outcome measures (MDS-UPDRS Part III and the MMSE). GluCer, GluSph, and LacCer and other sphingolipids were measured in PBMCs and plasma as biomarkers of pharmacological effect.

### Pharmacokinetic Results

Mean plasma concentrations were higher after the 60 mg dose than after the 10 mg dose at all time points (Day 7, Day 14, and on Day 28 before dosing and 1.5, 3, and 6 hours after the dose on Day 27).

### Pharmacodynamic Results

The fMRI and FDG-PET results were generally supportive of a pharmacodynamic effect of Compound A in subjects with GBA-PD. Compound A improved the default mode network (DMN), a large-scale network of different regions of the brain, assessed by fMRI. Differences between groups, small sample size, were not statistically significant.

Significant changes in PBMC glycolipid levels that were similar to those observed in Study LTI-291-003 were observed. The 60 mg dose group tended to show larger effects than the 10 mg dose group. Some changes in imaging measures (fMRI analyses) suggested a physiological effect of Compound A in subjects with GBA-PD. Plasma LacCer tended to decrease upon Compound A treatment, although no effect was observed in plasma GluCer or GluSph. Overall, results indicated that administration of Compound A during 28 days over placebo transiently increased the abundance of glycosphingolipid pathway intermediates measured in PBMC, which may indicate an increased GCase activity.

MDS-UPDRS III and MMSE scores on Day 28 were unchanged from (Day −1) in all subjects.

### Safety Results

No deaths, other SAEs, or severe AEs were reported. Most AEs were transient and resolved without sequelae. One subject in the 10 mg group withdrew from the study on Day 25 because of moderate back pain, which was considered by the investigator as unrelated to the study drug. No other moderate AEs were reported. Only muscle spasms (2 events, 1 subject in each dose group) and pollakiuria (2 events, 2 subjects in the 60 mg dose group) were reported in more than one subject. All other AEs were reported in only 1 subject. None of the AEs were considered related to the study treatment.

There were no clinically significant safety observations from vital sign measurements, laboratory safety assessments, physical examinations, or ECG parameters.

### Summary of Pharmacokinetics and Pharmacodynamics of Phase 1 Studies in GBA-PD Patients

In 2 Phase 1b, placebo-controlled, multiple-dose studies in subjects with GBA-PD (LTI-291-003 and LTI-291-004), the PK of Compound A were similar between healthy subjects and subjects with GBA-PD. Geometric mean CSF concentrations increased with increasing dose, and the CSF-to-plasma unbound drug concentration ratios were similar between dose levels after the 28th daily dose. There was an equivalent amount of unbound Compound A in plasma compared to CSF, indicating that Compound A is permeable into CSF and can cross the blood-brain barrier to reach GCase in brain tissue. Significant changes in PBMC glycolipid levels were observed in all dose groups of treated GBA-PD subjects (10, 30, and 60 mg/day), indicating a change in the flux of glycolipids due to Compound A treatment. The effects were generally greater in the subjects with major versus polymorphism GBA1 mutations. The changes were maximal through 2 weeks of treatment, and by 4 weeks, the magnitude of the effects was decreasing, possibly indicating that a new equilibrium in sphingolipid flux had been established. Some changes in imaging measures—such as functional magnetic resonance imaging (fMRI) and fluorodeoxyglucose positron emission tomography (FDG-PET)-suggested a normalizing physiological effect of Compound A in some subjects with GBA-PD. Overall, these data supported the hypothesis that Compound A engaged the GCase target and elicited changes in key glycosphingolipids secondary to increased flux through the system.

### Compound A—Thorough OTc Study (BIA 28-6156-105)

This was a Phase 1, randomized, double-blinded, placebo-controlled, single-dose, 4-period crossover study to evaluate the effect of Compound A on cardiac repolarization in healthy male and female subjects (aged 20 to 55 years). A total of 37 subjects (20 men and 17 women) were randomized to 1 of 12 treatment sequences and received a single oral dose 60 mg Compound A (n=34), 150 mg Compound A (n=36), placebo (as the placebo-control; n=35), and 400 mg moxifloxacin (as the positive control; n=34) under fed conditions on 4 occasions in a 4-way crossover manner. Objectives included evaluating the effect of single therapeutic (60 mg) and single supratherapeutic (150 mg) oral doses of Compound A on QT interval corrected for heart rate (HR [QTc]) based on the Fridericia correction (QTcF), evaluating the PK, safety, and tolerability of Compound A, correlating any observed effect on QTcF to plasma concentrations of Compound A, and confirming the effect of moxifloxacin on QTcF in healthy subjects for comparison to Compound A. Blood samples for PK measurements were taken through 72 hours after dosing during each period and continuous holter monitoring was performed for 24 hours after each dose.

### Pharmacokinetic Results

Median plasma Compound A Tₘₐₓ and geometric mean plasma Compound A t_{1/2} were similar between the 60 mg and 150 mg doses of Compound A, and exposure was higher following 150 mg Compound A compared to 60 mg Compound A (Table 6). Median plasma moxifloxacin Tₘₐₓ was 3.0 hours and geometric mean moxifloxacin t_{1/2} was 13.1 hours.

TABLE 6
Geometric Mean (Range) of Compound A and Moxifloxacin
Plasma Pharmacokinetic Parameters (PK Set)

| | 60 mg | | 150 mg | | 400 mg | |
|---|---|---|---|---|---|---|
| | Compound A | | Compound A | | Moxifloxacin | |
| Parameter | (N = 34) | | (N = 36) | | (N = 33) | |
| Cₘₐₓ (ng/mL) | 1617 | (958-3430) | 3598 | (2000-7150) | 1899 | (1130-2940) |
| Tₘₐₓ (h)a | 3.01 | (0.50-5.02) | 4.00 | (1.00-8.02) | 3.00 | (0.50-5.02) |
| AUC₀₋ₗₐₛₜ (ng · h/mL) | 41,005 | (22,081-72,948) | 101,853 | (56,352-194,899) | 27,508 | (17,033-36,661) |
| AUC_{0-inf} (ng · h/mL) | 52,073 | (23,438-106,049) | 131,488 | (59,681-266,812) | 28,212 | (17,157-38,479) |
| t_{1/2} (h) | 29.6 | (12.7-57.2) | 30.5 | (10.5-64.3) | 13.1 | (9.11-16.5) |

Abbreviations: AUC₀₋ₗₐₛₜ = area under the plasma concentration versus time curve (AUC) from time zero to the time of the last measurable concentration; AUC_{inf} = AUC from time zero extrapolated to infinity; Cₘₐₓ = maximum observed plasma drug concentration; Tₘₐₓ = time of maximum observed plasma drug concentration; t_{1/2} = terminal elimination half-life.

### Continuous Cardiac Monitoring and Safety Results

At the studied doses of 60 and 150 mg, BIA-28 had no clinically relevant effects on cardiac ECG parameters. The results of the concentration-QTc analysis suggested no effect on ΔΔQTcF exceeding 10 ms within the full observed range of plasma concentrations of Compound A up to approximately 7150 ng/mL. Moxifloxacin, the positive control, demonstrated a statistically significant effect on ΔΔQTcF above 5 msec as demonstrated by the concentration-QTc analysis, which demonstrated the sensitivity of the study to detect a positive effect on cardiac conduction parameters.

A total of 90 TEAEs were reported in 24 of 37 (64.9%) subjects during the study, and 88 of these TEAEs were transient and had resolved without sequelae by the follow-up visit. The two ongoing TEAEs were both mild in severity and considered unrelated to Compound A or moxifloxacin. No deaths or other SAEs were reported.

A total of 4 of 90 TEAEs, which were reported in 2 subjects, were considered related to Compound A 150 mg (headache and nausea reported by 1 subject, and increased ALT and increased GGT reported by another subject). Most TEAEs were mild in severity, 1 TEAE was of Grade 2 (moderate) severity (dental pulpitis considered unrelated to study drug), and 1 TEAE was of Grade 3 (severe) severity (hypersensitivity considered related to moxifloxacin). Two subjects were withdrawn from the study because of TEAEs: 1 subject because of increased ALT and increased GGT after receiving the 150 mg dose of Compound A, which were considered of Grade 1 severity and related to Compound A; and 1 subject because of hypersensitivity (Grade 3 in severity), pollakiuria (Grade 1 in severity), and nervousness (Grade 1 in severity) after receiving 400 mg moxifloxacin.

There were no clinically significant safety observations from vital sign measurements, physical examinations, safety ECG parameters, or laboratory values that were reported during the study and considered related to Compound A other than increased ALT and increased GGT in 1 subject after 150 mg Compound A.

### Compound A—Mass Balance Recovery and Metabolite Profiling Study (BIA 28-6156-106)

This was a Phase 1, single-dose, mass balance recovery study of ¹⁴C-Compound A in 1 cohort of healthy male subjects, aged 30 to 65 years. A total of 6 subjects received a single oral dose of 60 mg Compound A, containing not more than 3.7 MBq ¹⁴C, under fasted conditions. Objectives included determining the routes and rates of elimination of ¹⁴C-Compound A, identifying the structure of metabolites that account for more than 10% of circulating total radioactivity in plasma and total radioactive dose in urine and feces, exploring the PK of Compound A, evaluating the extent of distribution of TR into blood cells, and gathering additional safety and tolerability information for Compound A.

### Mass Balance and Metabolite Profiling Results

The mean (range) of radioactivity recovered in excreta over a 288-hour sampling period was approximately 93% (84.00% to 96.25%). The majority of total recovery was in the urine (mean: approximately 71%; range: 66.64% to 80.77%). Approximately 21% (range: 15.24% to 27.20%) of the total recovery was in the feces. Exposure to Compound A accounted for approximately 92% of circulating plasma total recovery based on AUC_{0-inf}, indicating that Compound A is the main circulating species in the plasma following oral administration.

Metabolite profiling and identification suggested that there are no systemic metabolites of Compound A present in excess of >10% AUC of drug-related exposure.

### Pharmacokinetic Results

The geometric mean plasma half-life of Compound A was 21.5 hours (range: 10.7 to 34.1 hours) after a single oral dose of 60 mg ¹⁴C BIA-28. Median Tₘₐₓ was 1.5 hours (range: 1.00 to 8.00 hours). The geometric mean (CV %) plasma Cₘₐₓ, AUC₀₋ₗₐₛₜ, and AUC_{0-inf} were 1750 ng/mL (38.3%), 41,700 ng·h/mL (10.4%) and 42,100 ng·h/mL (10.5%).

The geometric mean (geometric mean CV %) whole blood to plasma total radioactivity concentration ratios ranged from 0.542 (5.6%) to 0.562 (3.1%), indicating non-preferential distribution of total recovery to the cellular components of whole blood.

### Safety Results

No deaths, other SAEs, severe AEs, or discontinuations due to AEs were reported. In total, 3 (50.0%) subjects reported a total of 4 AEs. Two (33.3%) subjects reported mild headaches and 1 (16.7%) subject reported moderate tinea cruris. All AEs reported were assessed by the investigator as unrelated to the IMP and were self-limited and had resolved by the end of the study. There were no clinically significant safety observations from vital sign measurements, laboratory safety assessments, or ECG parameters.

### Drug-Drug Interaction Study (Study BIA-6156-107) (Compound A)

In a Phase 1, non-randomized, open-label, crossover, drug-drug interaction study of the effects of multiple-dose oral carbamazepine (a CYP3A4 inducer) on the single-dose PK of oral Compound A, 12 male and female subjects, aged 24 to 69 years, received 1) a single oral dose of 60 mg Compound A alone and with the 17th oral dose of carbamazepine ER, titrated to 300 mg twice daily.

Additionally, in a Phase 1, non-randomized, open-label, crossover, drug-drug interaction study of single-dose oral Compound A on the single-dose PK of oral carbidopa-levodopa, 12 male and female subjects, aged 24 to 69 years, received a single oral dose of carbidopa-levodopa 25/100 mg (25 mg of carbidopa and 100 mg of levodopa) IR alone and with a single oral dose of 60 mg Compound A.

Results of Study Compound A BIA 28-6156-107 are summarized as follows.

### Effect of Multiple-Dose Oral Carbamazepine on the Single-Dose PK of Compound A

Median Tₘₐₓ for Compound A concentrations was prolonged from 1-hour postdose for Compound A administered alone to 2 hours postdose for Compound A coadministered with carbamazepine. Reduced systemic exposure to Compound A was observed when Compound A was co-administered with the CYPP3A4 inducer, carbamazepine, relative to Compound A administered alone. Based on ratios of the geometric least squares means and associated 90% confidence intervals for the test-to-reference treatment comparisons, Compound A Cₘₐₓ was reduced by approximately 12% (point estimate: 0.877; 90% CI: 0.672 to 1.146), AUC₀₋ₗₐₛₜ was reduced by 63% (point estimate: 0.367; 90% CI 0.333 to 0.405), and AUC_{0-inf} was reduced by 64% (point estimate: 0.363; 90% CI: 0.328 to 0.402). The geometric mean t_{1/2} was 28 hours for Compound A administered alone and 12 hours for Compound A coadministered with carbamazepine.

### Effect of Single-Dose Compound A on the Single-Dose PK of Carbidopa-Levodopa

### Levodopa

Median Tₘₐₓ for levodopa concentrations was observed at 0.5-hour postdose when carbidopa-levodopa was administered alone and at 1-hour postdose when carbidopa-levodopa was coadministered with Compound A. Similar systemic exposure to levodopa was observed when carbidopa-levodopa was coadministered with Compound A and when carbidopa-levodopa was administered alone. Based on ratios of the geometric least squares means and associated 90% CIs for the test-to-reference treatment comparisons, the point estimates and 90% CIs for levodopa exposure parameters were 1.061 (0.918 to 1.227) for Cₘₐₓ, 1.048 (0.954 to 1.152) for AUC₀₋ₗₐₛₜ, and 1.035 (0.945 to 1.132) for AUC_{0-inf}. The geometric mean t_{1/2} for levodopa was similar under both treatment conditions (1.7 hours after carbidopa-levodopa alone and 1.4 hours after carbidopa-levodopa coadministered with Compound A).

### Carbidopa

Median Tₘₐₓ for carbidopa concentrations was observed at 2.5 hours postdose when carbidopa-levodopa was administered alone and at 2 hours postdose when carbidopa-levodopa was coadministered with Compound A. Mildly increased maximal exposure (by approximately 3%) and mildly decreased systemic exposure (by approximately 9%) to carbidopa were observed when carbidopa-levodopa was coadministered with Compound A relative to carbidopa-levodopa administered alone. Based on ratios of the geometric least squares means and associated 90% CIs for the test-to-reference treatment comparisons, the point estimates and 90% CIs for carbidopa exposure parameters were 1.031 (0.777 to 1.368) for Cₘₐₓ, and 0.909 (0.630 to 1.313) for AUC₀₋ₗₐₛₜ. The geometric mean t_{1/2} for carbidopa was similar under both treatment conditions (2 to 2.3 hours).

### Metabolite 3-O-methyldopa (3-OMD)

Median Tₘₐₓ for 3-OMD concentrations was observed at 6 hours postdose both when carbidopa-levodopa was administered alone and when carbidopa-levodopa was coadministered with Compound A. Similar systemic exposure to 3-OMD was observed when carbidopa-levodopa was coadministered with Compound A and when carbidopa-levodopa was administered alone. Based on ratios of the geometric least squares means and associated 90% CIS for the test-to-reference treatment comparisons, the point estimates and 90% CIs for 3-OMD exposure parameters were 1.056 (0.993 to 1.123) for Cₘₐₓ and 1.060 (1.010 to 1.112) for AUC₀₋ₗₐₛₜ. The geometric mean t_{1/2} for 3-OMD could not be calculated because most subjects did not have a terminal phase for their 12-hour PK profiles.

### Safety Results

Some TEAEs were observed. All TEAEs were transient and had resolved without sequelae by follow-up visit.

A total of 2 AEs (mild back pain, n=1; mild headache, n=1) were reported in 2 of the 12 subjects (16.7%) after administration of 60 mg of Compound A alone, and a total of 3 AEs (mild myalgia, n=1; mild headache, n=2) were reported in 3 of the 12 subjects (25.0%) after coadministration of 60 mg of Compound A with 300 mg of extended-release carbamazepine. No AEs were reported after coadministration of 60 mg of Compound A with 25 mg/100 mg of immediate-release carbidopa-levodopa. No SAEs were reported, and no TEAEs of ≥Grade 3 severity were reported. Most TEAEs were of Grade 1 (mild) severity. Only 4 TEAEs were of Grade 2 (moderate) severity: abdominal pain (considered related to Compound A or carbamazepine), hepatobiliary disease (considered related to Compound A, carbamazepine or carbidopa-levodopa), fall (considered unrelated to any of the study drugs), and constipation (considered related to carbamazepine and unrelated to Compound A or carbidopa-levodopa).

Two subjects were discontinued from the study treatment because of TEAEs. Decreased platelet count, which led to early discontinuation from the study in 1 subject, was reported as a Grade 1 (mild) TEAE and was considered related to carbamazepine. Viral infection (primary reason) and abdominal pain led to early discontinuation from the study in 1 subject. The viral infection was of Grade 1 (mild) severity and considered unrelated to any of the study drugs. Abdominal pain was of Grade 2 (moderate) severity and considered related to Compound A or carbamazepine, although the subject was receiving carbamazepine alone at the time of the event.

There were no clinically significant safety observations from vital sign measurements, physical examinations, ECG parameters, or the C-SSRS (Posner 2011). Other than the 1 subject who had a decreased platelet count and the 1 subject who had transient increases in liver enzymes, there were no other clinically significant laboratory values reported during the study.

### Drug-Drug Interaction Study (BIA-28-6156-109)

This was a Phase 1, non-randomized, open-label, 2-cohort, cross-over, DDI study in healthy male and female subjects. The study evaluated the effects of multiple doses of Compound A on the single-dose PK and safety of levodopa-carbidopa and of levodopa-benserazide.

In Cohort 1, a single oral dose 100 mg/25 mg levodopa-carbidopa (Sinemet®) IR was administered alone on Day 1. From Day 2 to Day 6, a single dose of 60 mg Compound A was administered. On Day 7, a single dose of 60 mg Compound A and a single dose of 100 mg/25 mg levodopa-carbidopa (Sinemet®) IR were administered.

In Cohort 2, a single oral dose 100 mg/25 mg levodopa-benserazide (Madopar®) IR was administered alone on Day 1. From Day 2 to Day 6, a single dose of 60 mg Compound A was administered. On Day 7, a single dose of 60 mg Compound A and a single dose of 100 mg/25 mg levodopa-benserazide (Madopar®) IR were administered.

For the Sinemet® portion, the carbidopa plasma PK samples were analyzed outside the demonstrated stability period, and the incurred sample reproducibility assessment did not meet the acceptance criteria. Therefore, the carbidopa results were not described in this CSR.

For the Madopar® portion, the benserazide plasma PK results were not described in this CSR since all benserazide concentrations (except 2) measured in this study were below the LLOQ. Statistical analysis of DDI was performed for exploratory purposes only. Hence the application of default bioequivalence ranges should be interpreted in this context. In both portions, an effect of multiple doses of Compound A on the single-dose PK of levodopa could not be excluded, as the upper limits of the 90% CIs for the AUC ratios were not contained within the default no-effect boundary of 80% to 125%. Future research may be needed to confirm a potential clinical DDI. Nevertheless, the potential effect of Compound A on the PK of levodopa seems to be limited (AUC increases of ~13% and ~24% for Cohorts 1 and 2, respectively). Per the Food and Drug Administration Guidance for Drug-drug Interactions (“Clinical Drug Interaction Studies—Cytochrome P450 Enzyme- and Transporter-Mediated Drug Interactions, Guidance for Industry”. U.S. Department of Health and Human Services, Food and Drug Administration, Center for Drug Evaluation and Research (CDER). January 2020, Clinical Pharmacology), this would be a minimal effect as AUC increases between >1.25 fold and <2 fold are considered a weak DDI effect.

### Levodopa—Sinemet® Portion: Effects of Multiple Doses of Compound a on Single-Dose

### Plasma PK of Levodopa and 3-OMD

For levodopa, the 90% CIs of the ratios of the geometric least squares mean were 1.231 (1.006 to 1.508) for Cₘₐₓ, 1.130 (1.004 to 1.272) for AUC₀₋ₜ, and 1.132 (1.007 to 1.272) for AUC_{0-inf} after coadministration of Sinemet®/Compound A compared to administration of Sinemet® alone. These results suggested an increased maximal exposure to levodopa by approximately 23% for Cₘₐₓ (based on the estimate of 1.231), and an increased systemic exposure to levodopa by approximately 13% for AUC0-t (based on the estimate of 1.130) and AUC0-inf (based on the estimate of 1.132) compared to administration of Sinemet® alone.

However, there was no effect of Compound A on Sinemet® with regard to 3-OMD PK based on the 90% CIs of the ratios of the geometric least squares mean Cₘₐₓ (estimate of 1.077; 90% CI ranging from 0.980 to 1.184) and AUC₀₋ₜ (estimate of 1.065; 90% CI ranging from 0.979 to 1.159). The geometric mean t½ values of levodopa were similar after combined administration of Compound A and Sinemet® (1.59 hours) compared to Sinemet® alone (1.69 hours), which suggests clearance of levodopa is not impacted by Compound A coadministration. It was expected that steady state concentrations of Compound A would have been reached after 6 days of dosing since t½ was 25 hours to 35 hours after multiple doses in Trial LTI-291-002. However, based upon visual inspection of the geometric mean trough plasma concentration-time curve, steady state concentrations of Compound A appeared not to have been reached after 6 days of dosing with 60 mg Compound A (from Days 2 to 7), as trough concentrations on these days were still increasing. However, the curve was flattening by Day 7 indicating that steady state Compound A concentrations would soon be reached.

Sinemet ® Portion: Summary Statistics (Geometric Mean [Range])
of Levodopa and 3-OMD Plasma PK Parameters (PK Set)

| | | |
|---|---|---|
| | Sinemet ® | Sinemet ® + |
| | Alone | Compound A |
| Parameter | (N = 12) | (N = 12) |

Levodopa

| | | | | |
|---|---|---|---|---|
| Cmax (ng/mL) | 861 | (472-1780) | 1060 | (526-2440) |
| tmax (h)^{a} | 1.00 | (0.50-1.50) | 0.50 | (0.50-3.00) |
| AUC0-t (ng · h/mL) | 1534 | (1025-2387) | 1734 | (888-2862) |
| AUC0-inf (ng · h/mL) | 1563 | (1047-2405) | 1768 | (903-2908) |
| t1/2 (h) | 1.69 | (1.44-2.10) | 1.59 | (1.28-2.15) |
| CL/F (L/h) | 64.0 | (41.6-95.5) | 56.6 | (34.4-111) |
| Vz/F (L) | 156 | (101-228) | 130 | (75.0-244) |

3-OMD

| | | | | |
|---|---|---|---|---|
| Cmax (ng/mL) | 426 | (217-675) | 459 | (201-733) |
| tmax (h)^{a} | 6.00 | (4.00-6.22) | 5.00 | (4.00-6.00) |
| AUC0-t (ng · h/mL) | 4212 | (2171-6417) | 4485 | (2043-7112) |

| | | |
|---|---|---|
| AUC0-inf (ng · h/mL) | —^{b} | —^{c} |
| t1/2 (h) | 20.3 (18.7-22.4) | 13.9 (8.37-17.1) |
| | (n = 3)^{d} | (n = 6)^{d} |

% AUCextra = percentage of estimated part for the calculation of AUC0-inf;
3-OMD = 3-O-methyldopa;
N = numberof subjects;
n = number of subjects for the parameter;
PK = pharmacokinetic
The n was 12, except when otherwise specified
^{a}For tmax the median (range) is presented instead of geometric mean (range)
^{b}For 3 subjects, the criteria of % AUCextra ≤20% were not met, and for 9 subjects, AUC0-inf could not be estimated
^{c}For 6 subjects, the criteria of % AUCextra ≤20% were not met, and for 6 subjects, AUC0-inf could not be estimated
^{d}As half-lives are much larger than the range over which they were calculated, they should be considered unreliable.

Sinemet ® Portion: Statistical Analysis of Drug-Drug Interaction Between
Compound A and Sinemet ® (PK Set)

| | Geometric Least Squares Mean | | Ratio Test/Reference | | | |
|---|---|---|---|---|---|---|
| | | Sinemet ® + | Sinemet ® | | | |
| | | Compound A | Alone | | 90% | CI |
| Analyte | PK Parameter | Test | Reference | Estimate | Lower | Upper |
| Levodopa | Cmax (ng/mL) | 1060 | 861 | 1.231 | 1.006 | 1.508 |
| | AUC0-t (ng · h/mL) | 1734 | 1534 | 1.130 | 1.004 | 1.272 |
| | AUC0-inf (ng · h/mL) | 1768 | 1563 | 1.132 | 1.007 | 1.272 |
| 3-OMD^{a} | Cmax (ng/mL) | 459 | 426 | 1.077 | 0.980 | 1.184 |
| | AUC0-t (ng · h/mL) | 4485 | 4212 | 1.065 | 0.979 | 1.159 |

3-OMD = 3-O-methyldopa;
AUC = area under the plasma concentration-time curve;
CI = confidence interval;
In = natural logarithm;
PK = pharmacokinetic
Note:
The Cmax and AUC analyses were performed on In-transformed parameters using a linear mixed-effects model with treatment as a fixed effect and subject as a random effect
^{a}For 3-OMD, AUC0-inf values could not be estimated or not estimated accurately.

Sinemet ® Portion: Summary Statistics (Geometric Mean
[Range]) of Compound A Plasma PK Parameters (PK Set)

| | Compound A | |
|---|---|---|
| | Alone | |
| Parameter | (N = 12) | |
| Cmax (ng/mL) | 1942 | (1130-2710) |
| tmax (h)^{a} | 2.00 | (1.00-4.00) |
| AUC0-t (ng · h/mL) | 22,696 | (16,586-31,082) |
| AUC0-24 (ng · h/mL) | 22,801 | (16,675-31,209) |

N = number of subjects;
PK = pharmacokinetic
^{a}For tmax the median (range) is presented instead of geometric mean (range)

### Levodopa—Madopar® Portion: Effects of Multiple Doses of Compound A on Single-Dose

### Plasma PK of Levodopa and 3-OMD

For levodopa, the 90% CIs of the ratios of the geometric least squares mean were 1.330 (1.124 to 1.575) for Cₘₐₓ, 1.238 (1.146 to 1.337) for AUC0-t, and 1.237 (1.146 to 1.335) for AUC0-inf after coadministration of Madopar®/Compound A compared to administration of Madopar® alone. These results suggested an increased maximal exposure to levodopa by approximately 33% for Cₘₐₓ (based on the estimate of 1.330), and an increased systemic exposure to levodopa by approximately 24% for AUC0-t (based on the estimate of 1.238) and AUC0-inf (based on the estimate of 1.237) compared to administration of Madopar® alone.

However, there was no effect of Compound A on Madopar® with regard to 3-OMD PK based on the 90% CIs of the ratios of the geometric least squares mean Cₘₐₓ (estimate of 1.131; 90% CI ranging from 1.086 to 1.178) and AUC0-t (estimate of 1.115; 90% CI ranging from 1.075 to 1.158). The geometric mean t½ values of levodopa were similar after combined administration of Compound A and Madopar® (2.45 hours) compared to Madopar® alone (2.26 hours), which suggests clearance of levodopa is not impacted by Compound A coadministration.

It was expected that steady state concentrations of Compound A would have been reached after 6 days of dosing since t½ was 25 hours to 35 hours after multiple doses in Trial LTI-291-002. However, based upon visual inspection of the geometric mean trough plasma concentration-time curve, steady state concentrations of Compound A appeared not to have been reached after 6 days of dosing with 60 mg Compound A (from Days 2 to 7), as trough concentrations on these days were still increasing. However, the curve was flattening by Day 7 indicating that steady state Compound A concentrations would soon be reached.

Based on statistical analysis, there was an increased maximal systemic exposure to levodopa by approximately 23% for Cₘₐₓ, and an increased systemic exposure to levodopa by approximately 13% for both AUC0-t and AUC0-inf after combined administration of Compound A and Sinemet® compared to Sinemet® alone. However, there was no effect of Compound A on Sinemet® based on the plasma exposure PK parameters of 3-OMD.

Based on statistical analysis, there was an increased maximal systemic exposure to levodopa by approximately 33% for Cₘₐₓ, and an increased systemic exposure to levodopa by approximately 24% for both AUC0-t and AUC0-inf after combined administration of Compound A and Madopar® compared to Madopar® alone. However, there was no effect of Compound A on Madopar® based on the plasma exposure PK parameters of 3-OMD.

The geometric mean t½ values of levodopa were similar after combined administration of Compound A and Sinemet® compared to Sinemet® alone, which suggests clearance of levodopa is not impacted by Compound A. The geometric mean t½ values of levodopa were similar after combined administration of Compound A and Madopar® compared to Madopar® alone, which suggests clearance of levodopa is not impacted by Compound A coadministration.

Steady state concentrations of Compound A appeared not to have been reached at Sinemet®/Compound A coadministration on Day 7 or Madopar®/Compound A coadministration on Day 7.

Madopar ® Portion: Summary Statistics (Geometric Mean [Range])
of Levodopa and 3-OMD Plasma PK Parameters (PK Set)

| | | |
|---|---|---|
| | Madopar ® | Madopar ® + |
| | Alone | Compound A |
| Parameter | (N = 12) | (N = 11) |

Levodopa

| | | | | |
|---|---|---|---|---|
| Cmax (ng/mL) | 1239 | (667-3420) | 1699 | (972-3020) |
| tmax (h)^{a} | 0.77 | (0.50-2.00) | 0.50 | (0.50-1.00) |
| AUC0-t (ng · h/mL) | 1935 | (1449-4339) | 2450 | (1607-4204) |
| AUC0-inf (ng · h/mL) | 1964 | (1475-4365) | 2485 | (1633-4239) |
| t1/2 (h) | 2.26 | (1.65-3.01) | 2.45 | (1.62-3.54) |
| CL/F (L/h) | 50.9 | (22.9-67.8) | 40.2 | (23.6-61.2) |
| Vz/F (L) | 166 | (61.4-294) | 142 | (64.8-254) |

3-OMD

| | | | | |
|---|---|---|---|---|
| Cmax (ng/mL) | 588 | (243-1280) | 672 | (289-1270) |
| tmax (h)^{a} | 4.00 | (1.00-8.00) | 4.00 | (1.00-6.07) |
| AUC0-t (ng · h/mL) | 5983 | (2534-12,942) | 6749 | (3093-13,042) |

| | | |
|---|---|---|
| AUC0-inf (ng · h/mL) | —^{b} | —^{c} |
| t1/2 (h) | 16.4 (9.08-23.2) | 18.1 (10.7-32.2) |
| | (n = 8)^{d} | (n = 9)^{d} |

% AUCextra = percentage of estimated part for the calculation of AUC0-inf;
3-OMD = 3-O-methyldopa;
N = numberof subjects;
n = number of subjects for the parameter;
PK = pharmacokinetic
For Madopar ® alone, the n was 12, except when otherwise specified
For Madopar ® + Compound A, the n was 11, except when otherwise specified
^{a}For tmax the median (range) is presented instead of geometric mean (range)
^{b}For 8 subjects, the criteria of % AUCextra ≤20% were not met, and for 4 subjects, AUC0-inf could not beestimated
^{c}For 9 subjects, the criteria of % AUCextra ≤20% were not met, and for 2 subjects, AUC0-inf could not be estimated
^{d}As half-lives are much larger than the range over which they were calculated, they should be considered unreliable

Madopar ® Portion: Statistical Analysis of Drug-Drug Interaction
Between Compound A and Madopar ® (PK Set)

| Geometric Least Squares Mean | |
|---|---|
| Madopar ® + | Madopar ® |

| | | | | |
|---|---|---|---|---|
| Analyte | PK Parameter | Compound A | Alone | Ratio Test/Reference |

Analyte PK Parameter

| | | | | | 90% | CI |
|---|---|---|---|---|---|---|
| | | Test | Reference | Estimate | Lower | Upper |
| Levodopa | Cₘₐₓ (ng/mL) | 1648 | 1239 | 1.330 | 1.124 | 1.575 |
| | AUC₀₋ₜ (ng · h/mL) | 2395 | 1935 | 1.238 | 1.146 | 1.337 |
| | AUC_{0-inf} (ng · h/mL) | 2429 | 1964 | 1.237 | 1.146 | 1.335 |
| 3-OMDª | Cₘₐₓ (ng/mL) | 665 | 588 | 1.131 | 1,086 | 1.178 |
| | AUC₀₋ₜ (ng · h/mL) | 6673 | 5983 | 1.115 | 1.075 | 1.158 |

3-OMD = 3-O-methyldopa;
AUC = area under the plasma concentration-time curve;
CI = confidence interval;
CI = confidence interval;
In = natural logarithm;
PK = pharmacokinetic
Note:
The Cₘₐₓ and AUC analyses were performed on In-transformed parameters using a linear mixed-effects model with treatment as a fixed effect and subject as a random effect
^{a}For 3-OMD, AUC_{0-inf} values could not be estimated or not estimated accurately.

Madopar ® Portion: Summary Statistics (Geometric Mean
[Range]) of Compound A Plasma PK Parameters (PK Set)

| | Compound A | |
|---|---|---|
| Parameter | Alone(N = 12) | |
| Cmax (ng/mL) | 2033 | (1520-2910) |
| tmax (h)^{a} | 2.00 | (1.00-2.02) |
| AUC0-t (ng · h/mL) | 22,786 | (12,353-28,955) |
| AUC0-24 (ng · h/mL) | 24,017 | (18,217-29,100) |

N = number of subjects;
PK = pharmacokinetic
^{a}For tmax the median (range) is presented instead of geometric mean (range)

Overall, the results of this study suggest that Compound A can be co-administered with levodopa-carbidopa or levodopa-benserazide; however, a potentially clinically meaningful DDI in PD patients cannot be excluded.

### Drug-Drug Interaction Study (BIA 28-6156-110)

This was a Phase 1, non-randomized, open-label, 2-cohort, cross-over, DDI study in healthy male and female subjects. The study evaluated the effects of multiple doses of Compound A on the single-dose PK and safety of rosuvastatin and metformin.

In Cohort 1, a single oral dose of 10 mg rosuvastatin was administered alone on Day 1. No drug was administered on Day 2. From Day 3 to Day 8, a single daily dose of 60 mg Compound A was administered. On Day 9, a single dose of 60 mg Compound A and a single dose of 10 mg rosuvastatin were administered.

In Cohort 2, a single oral dose 500 mg metformin was administered alone on Day 1. From Day 2 to Day 7, a single daily dose of 60 mg Compound A was administered. On Day 8, a single dose of 60 mg Compound A and a single dose of 500 mg metformin were administered.

Statistical analysis of DDI was performed for exploratory purposes only. Hence the application of default bioequivalence ranges below should be interpreted in this context.

In the rosuvastatin portion, an effect of multiple doses of Compound A on the single-dose PK of rosuvastatin could not be excluded as both the upper and lower limits of the 90% CIs for the AUC ratios were not contained within the default no-effect boundary of 80% to 125%. The effect of Compound A on the PK of rosuvastatin was about 3-fold increase with AUC incrementing from 304% to 344%. In the metformin portion the 90% CIs of the of metformin AUC ratios were contained within the default no-effect boundary of 80% to 125%, suggesting no effect of multiple oral doses of Compound A on metformin PK following a single oral dose of metformin.

Truncated PK profiles for rosuvastatin over 48 hours and for metformin over 24 hours are regarded efficient to allow characterisation and comparison of rate and extent of absorption in either combined administration with Compound A or alone. However, parameters related to elimination bear a higher degree of uncertainty and should be interpreted with caution.

### Rosuvastatin Portion: Effects of Multiple Doses of Compound A on Single-Dose Plasma Pharmacokinetics of Rosuvastatin

The 90% CIs of the ratios of rosuvastatin geometric least squares mean Cmax (estimate of 3.467; 90% CI ranging from 3.141 to 3.826), AUC0-t (estimate of 3.437; 90% CI ranging from 3.151 to 3.750), and AUC0-inf (estimate of 3.041; 90% CI ranging from 2.759 to 3.352) were not contained within the default no-effect boundary of 80% to 125%. Thus, for Compound A co-administration an increased maximal exposure to rosuvastatin by 347% for Cmax (based on the estimate of 3.467), an increased systemic exposure to rosuvastatin by 344% for AUC0-t (based on the estimate of 3.437), and an increased systemic exposure to rosuvastatin by 304% for AUC0-inf (based on the estimate of 3.041) compared to administration of rosuvastatin alone, was observed. It was expected that steady state concentrations of Compound A would have been reached after 6 days of dosing. However, based upon visual inspection of the geometric mean trough plasma concentration-time curve, steady state concentrations of Compound A appeared not to have been reached after 6 days of dosing with 60 mg Compound A (from Days 3 to 9), as trough concentrations on these days were still increasing.

### Metformin Portion: Effects of Multiple Doses of Compound A on Single-Dose Plasma Pharmacokinetics of Metformin

The 90% CIs of the ratios of metformin geometric least squares mean Cmax (estimate of 0.950; 90% CI ranging from 0.877 to 1.028), AUC0-t (estimate of 0.938; 90% CI ranging from 0.881 to 1.000), and AUC0-inf (estimate of 0.940; 90% CI ranging from 0.882 to 1.002) were contained within the default no-effect boundary of 80% to 125, suggesting no effect of multiple oral doses of Compound A on metformin PK following a single oral dose of metformin. It was expected that steady state concentrations of Compound A would have been reached after 6 days of dosing. However, based upon visual inspection of the geometric mean trough plasma concentration-time curve, steady state concentrations of Compound A appeared not to have been reached after 6 days of dosing with 60 mg Compound A (from Days 2 to 8), as trough concentrations on these days were still increasing.

### Example 2

The in vitro measurement of GCase activity in human brain homogenates was used to correlate the exposure and GCase activity and to estimate the increase in GCase activity in the brain of GBA-PD patients at Cₘᵢₙ at steady state (C_{b,min,ss}) across different dose levels. The relationship between concentration observed in brain and CSF in rat was used to obtain the brain concentrations of Compound A in humans, assuming the same relationship conserved among mammalian species. The range of human estimated C_{b,min,ss} were superimposed to the corresponding concentration from in vitro study with human brain homogenates to obtain the GCase activation by Compound A.

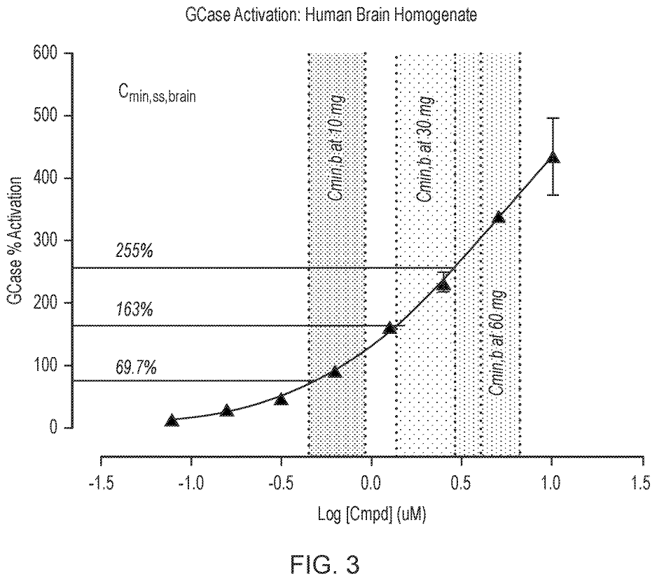

It was found that the minimum GCase activity in GBA-PD patients across dose ranges of 10, 30 and 60 mg should be between 69.7-123%, 163-306% and 255-376%, respectively (FIG. 3). Based on the GCase activity, it was concluded that since the minimum GCase activity in GBA-PD patients during dosing interval (24 h) at 10 mg (69.7%) is approximately 2 times higher than effective GCase activity, all three dose levels should be associated with effect.

In vitro GCase Brain Homogenate Activity Assays

The activity of Compound A in the GCase assay was determined using 4-MUG (1 mM) as substrate and brain homogenate (5 μg/well) as enzyme source.

A 500 μl aliquot of a 1.0 mg/ml solution of phosphatidylserine (PS, Sigma P7769) in chloroform was evaporated under a stream of nitrogen for 1 hour. The lipid film was dissolved with 4 minutes of vigorous vortexing in 41 ml of 176 mM K₂HPO₄/50 mM citric acid pH 4.7 containing 7.7 μl of triton X-100, resulting in a mixed micellar preparation with a composition of 0.32 mM triton and 4.6 mol % PS.

4-methylumbelliferyl-beta-D-glucopyranoside (4-MUG, ACROS-337025000) was dissolved in the micellar solution to a final concentration of 2 mM for use as the reaction substrate. The micellar/homogenate solution was prepared by adding 3.2 ml Triton X-100/phosphotidylserine mixed micelles containing 400 μg/mL brain homogenate. A 1.6 mL portion of this mixture received 32 μl conduritol B epoxide (CBE, 100 mM stock in DMSO) for final concentration of 2 mM CBE. The remaining 1.6 mL portion received 32 μl DMSO as solvent control. Compound A was diluted with DMSO from a 10 mM stock to the desired assay concentrations, and 0.45 μl of compound in DMSO was added to 75 μl of micellar/homogenate solution (+/−CBE).

After a 30 minute pre-incubation at room temperature, the reaction was initiated by combining 25 μl of substrate solution with 25 μl of compound/GCase/homogenate mixture. The reaction proceeded for 30 minutes at room temperature for Rat, Cyno and Dog brain homogenates, 60 minutes for Mouse and Human brain homogenate. The reaction was stopped by adding 150 μl of 1M glycine, pH 12.5. The endpoint fluorescence intensity of the reaction was measured at excitation 365 nm, emission 440 nm on a SpectraMax i3 instrument (Molecular Devices). Compound activity was expressed relative to a DMSO control. GCase activity in the CBE containing samples was subtracted from GCase activity in DMSO containing samples to obtain GBA1 activity plotted on the dose response curves.

Method for Quantification of Compound A in human CSF

To measure the amount of compound A in the CSF, CSF was diluted 1:1 (v/v) with 0.2% Bovine Serum Albumin and sample processing was performed by means of protein precipitation/dilution using a sample volume of 50.0 μL and compound A as the internal standard. Separation between metabolites and interfering endogenous compounds was achieved by HPLC using a Shim-pack XR-ODS column (3.0×50 mm, 2.2 μm particles), at a temperature of 60° C., using 0.1% formic acid in water as mobile phase A, acetonitrile as mobile phase B and operating isocratically at 55% B followed by a step gradient at 85% B, at a flow rate of 0.900 mL/min.

An API4000 mass spectrometer equipped with a turbo ion spray source was used for detection in positive ion mode. Quantification was based on multiple reaction monitoring (MRM) of the transitions of m/z 359.2-174.2 for LTI-291 and m/z 365.3-180.2 for its internal standard. A linear calibration curve with a 1/x2 weighting factor was used ranging from 0.0500 to 100 ng/mL in CSF for LTI-291 (0.0250-50.0 ng/mL in CSF: 0.2% BSA 1:1 (v/v)).

The results were plotted and analysed using Analyst version 1.6.2 (AB Sciex, Concord, Canada).

### Example 3

This study is a Phase 2, randomized, double-blind, placebo-controlled, multiple oral dose study of Compound A in subjects (aged 35 to 80 years, inclusive) with GBA-PD. The subjects have a clinical diagnosis of PD for at least 1 year and for no longer than 7 years before initiation of screening (for Part A), as confirmed by a neurologist using the MDS Criteria for Parkinson's Disease, have a modified Hoehn and Yahr score 2.5, have a score of ≥22 on the Montreal Cognitive Assessment (MoCA), and are receiving symptomatic treatment for PD. In some embodiments, the subjects do not have moderate (or severe) motor complications as assessed by a score 3 in any of the subitems of the MDS-UPDRS Part IV, and the subjects do not have clinically significant psychosis in the clinical judgment of the investigator. The objectives of this study are to investigate the efficacy, safety, tolerability, pharmacodynamics and PK of once-daily oral administration of Compound A.

Genetic screening of the subjects is performed using blood collected (preferably whole blood samples). Subjects are screened by sequencing of the full GBA1 gene, with specifications of any PD risk-associated variants that are identified. Subjects are also screened for the presence or absence of PD risk-associated variants in the LRRK2 (Leucine-rich repeat kinase 2) gene. Subjects who have an LRRK2 pathogenic variant are not eligible for Part B (Double-Blind Treatment) of the study.

Subjects that have a pathogenic variant in the glucocerebrosidase 1 (GBA1) gene (GBA-PD) (for example, see Appendix 1) and have been on a stable dose of PD medication for at least 30 days (at least 60 days for rasagiline) before commencing treatment are included in the next stage of the trial. In addition, subjects with the following are excluded from further treatment:
The subject has Gaucher's disease (GD), as defined by clinical signs and symptoms (i.e., hepatosplenomegaly, cytopenia, skeletal disease), and/or a medical history of marked deficiency of GCase plasmatic activity (preferably GCase whole blood activity) compatible with GD.
The subject is homozygous for a GBA1 pathogenic variant that is known to be associated with GD or compound heterozygous for 2 alleles that are known to be associated with GD.
The subject carries a known PD-associated LRRK2 pathogenic variant (for example, see Appendix 2).
The subject has atypical or secondary parkinsonism by medical history or in the opinion of the investigator. Atypical parkinsonism includes, but is not limited to, diagnoses of progressive supranuclear palsy, cortico-basal syndrome, and multiple system atrophy. Secondary parkinsonism includes drug-induced, toxin-induced, postinfectious, posttraumatic, or vascular parkinsonism.
The subject is using a strong CYP3A4 modulator at the time of screening for Part B.
The subject is using a breast cancer resistance protein (BCRP) substrate (e.g. pravastatin, rosuvastatin, glyburide) at the time of screening for Part B.
(Optionally) The subject has used any of the following medications within 60 days before Baseline: typical or atypical antipsychotics (including, but not limited to, clozapine, pimavanserin, olanzapine, quetiapine, risperidone, and aripiprazole); metoclopramide; prochlorperazine; methyldopa; tetrabenazine; deutetrabenazine; valbenazine; reserpine; or a prior history or continuation or initiation during the study of ambroxol at doses >120 mg/day.

Subjects are randomly assigned to three groups who receive i) Compound A 10 mg, once daily, oral administration, ii) Compound A 60 mg, once daily, oral administration, or iii) Placebo, once daily, oral administration. Subjects also receive their usual PD medications throughout the study. Randomization is stratified by major/severe (e.g., heterozygous for N370S, D409H, H255Q, D140H, G202R, L324P, I260T, L444P, A190T, R120W; homozygous for T369M or E326K) or minor/mild (e.g., heterozygous for T369M or E326K) PD risk-associated variant. Alternatively, randomization is stratified by severity as a mild or severe GBA1 variant as described in Parlar, 2023 (also see Appendix 1). Subjects who have other PD risk-associated variants are classified as either major/severe or minor/mild based on emerging data and are randomized accordingly.

Subjects receive treatment for up to 78 weeks followed by a 30-day (or 4 week) safety follow-up. Efficacy, safety, tolerability, pharmacodynamic, and PK are evaluated at study visits conducted at Baseline and at Weeks 4, 12, 26, 39, 52, 65, and 78. Study personnel contact the subjects before each scheduled study visit to remind them to take their last dose of non-investigational PD medication >10 hours before each study visit. Subjects may restart their non-investigational PD medication after the study visit assessments are completed. Study visits are rescheduled for subjects who take their non-investigational PD medication <10 hours before a scheduled study visit.

Study visits are scheduled at approximately the same time of day to ensure that the assessment scales are completed at consistent times during the study. Every effort is made to have the same rater perform the same assessments for an individual subject throughout the study. Where possible, efficacy assessments are performed first followed by safety assessments and then by blood draws.

Subjects may require unscheduled visits for any adjustment of non-investigational PD medication. In such situations the last dose of non-investigational PD medication is taken ≥10 hours before each of the unscheduled visits where assessment of MDS-UPDRS Part I-IV, CGI-C and PGI-C are performed before any adjustment of non-investigational PD medication. Subjects may restart their non-investigational PD medication after the study visit assessments are completed.

The following criteria are assessed at regular intervals throughout the study:
Movement Disorder Society-Unified Parkinson's Disease Rating Scale (MDS-UPDRS) part I, II, III and IV
Clinical Global Impression-Change (CGI-C) and Severity (CGI-S)
Patient Global Impression-Change (PGI-C) and Severity (PGI-S)
Levodopa-Equivalent Daily Dosage (LEDD)
Modified Hoehn and Yahr score.
Parkinson's Disease Cognitive Rating Scale (PD-CRS)
39-Item Parkinson's Disease Questionnaire (PDQ-39)
EuroQol 5 Dimension 5 Level scale (EQ-5D-5L)
Gait speed

In addition, the PD biomarkers and GCase activity in whole blood samples are measured at baseline and week 78 (preferably baseline and weeks 4, 26, 52 and 78). This may include biomarkers of lysosomal activity (e.g., glucosylsphingosine (GluSph) and/or glucosylceramide (GluCer)). Methods of measuring lysosomal activity (e.g., GluSph and/or GluCer) are described above. A method of measuring GCase activity is described below. Magnetic resonance imagining (MRI) scans are performed at baseline and week 78 for a cohort of GBA-PD subjects.

Efficacy, safety, tolerability, and pharmacodynamic assessments are conducted at study visits that are conducted at Baseline and at Weeks 4, 12, 26, 39, 52, 65, and 78. A telephone contact is conducted at Week 8 to inquire about the occurrence of any adverse events (AEs) and any changes in concomitant medications.

Blood for determination of plasma concentrations of Compound A for a population PK analysis is obtained from all subjects before administration of the first dose of IMP at the study site at the Baseline visit and during the Weeks 4, 12, 26, 39, 52, 65, and 78 visits. Subjects are instructed to take their dose of IMP in the morning before the Weeks 4, 12, 26, 39, 52, 65, and 78 visits. The time of collection of the PK blood sample is recorded.

A baseline magnetic resonance imaging (MRI) scan is obtained during the Screening Period from subjects who are considered eligible and who provide consent to participate in the optional MRI Sub-study. The baseline MRI is obtained within the 35-day screening window and is obtained after all other screening assessments have confirmed that the subject is eligible for the study. A posttreatment MRI is obtained at Week 78 for subjects who participate in the MRI Sub-study; a window of −2 weeks to +1 day is allowed for the Week 78 MRI.

Plasma concentrations are summarized at each time point, and no PK parameters are estimated. The PD evaluations include fMRI measures; FDG-PET to determine the metabolic rate of glucose in specific regions of the brain; and functional outcome measures (MDS-UPDRS Part III and the MMSE). GluCer, GluSph, and LacCer and other sphingolipids are measured in PBMCs and plasma as biomarkers of pharmacological effect.

The preferred method of measuring GCase activity in whole blood is described below, and also in Totorelli, 2016, which is incorporated herein by reference:

A 5-plex cocktail assay for GCase, acid a-glucosidase (GAA), galactocerebrosidase (GALC), a-galactosidase A (GLA), and a-L-iduronidase (IDUA) activity is prepared. Vials containing the substrate (S) and internal standards (IS) for GCase and GAA are reconstituted with methanol and transferred to the GALC S+IS vial followed by evaporation. The GLA S+IS vial is reconstituted with 1.8 mL of 96 g/L sodium taurocholate in water and then transferred to the dry GALC S+IS vial, which is then heated to 60° C. to dissolve all solids. To the GALC S+IS vial the following reagents are added: 0.3 mL of 0.8 mmol/L acarbose in water (GAA inhibitor) and 2.88 mL of 1 mol/L N-acetylgalactosamine (GLA inhibitor) in buffer (0.2 mol/L sodium phosphate+0.1 mol/L sodium citrate, pH=4.4). The IDUA S+IS vial is then reconstituted with 12.52 mL of buffer (0.2 mol/L sodium phosphate+0.1 mol/L sodium citrate, pH=4.4) and 0.5 mL of 3 mmol/L D-saccharic acid 1,4-lactone in water. The reconstituted IDUA S+IS vial is transferred to the GALC S+IS vial. An acid sphingomyelinase (ASM) cocktail is prepared separately by reconstituting the ASM S+IS vial with 0.15 mL of 96 g/L sodium taurocholate in water and then adding 17.85 mL of buffer (0.85 mol/L sodium acetate+0.604 mmol/L zinc chloride, pH=5.7). After mixing, 1-mL aliquots of these reagents are stored at −20° C. for up to 2 months.

Three 3-mm discs are excised from a dried blood spot (DBS) prepared by spotting blood on Whatman 903 filter paper and allowing to dry for at least 3 h at ambient conditions and placed into individual microtiter plates. The first disc is treated with 30 μL of ASM cocktail, the second one with 30 μL 5-plex cocktail containing S and IS for GCase, GAA, GLA, GALC, and IDUA. The 2 enzyme plates are sealed, centrifuged for 2 min at 493 g, and incubated for 19 h at 37° C. The third disc is extracted with 300 μL of methanol containing 12 ng/mL d₄-C₂₆ lysophosphadildicholines (LPC) as described by Turgeon et. al (Turgeon, 2015). The extract is then evaporated under heated nitrogen, reconstituted in 130 μL of mobile phase (800 mL methanol/200 mL water with 5 mmol/L ammonium formate) and stored refrigerated. Following the 19-h incubation, the 5-plex and ASM reactions are stopped by adding 200 μL of 1:1 ethyl acetate:rmethanol to each plate. The 5-plex and ASM plates are combined into a single deep-well plate and liquid-liquid extraction is performed by adding 400 μL of ethyl acetate and then 400 μL of water to each well. The plate is sealed and centrifuged at 493 g for 2 min, and 150 μL of the organic layer is transferred to a new plate, evaporated under nitrogen, and reconstituted in 150 μL of 19:1 ethyl acetate:methanol. The reconstituted extract is added to a silica-containing filter plate that had been washed previously with 200 μL of 19:1 ethyl acetate. The sample is moved through the filter plate via positive pressure and the plate is eluted with an additional 200 μL of 19:1 ethyl acetate. The 350 μL of eluent collected is evaporated under a stream of nitrogen and then reconstituted with the stored (X-adrenoleukodystrophy) ALD plate. The samples are subjected to flow injection tandem mass spectrometry (FIA-MS/MS) analysis. The total time to process a plate of 96 samples is 60 and 70 min, respectively, before and after the 19 h incubation.

MS/MS procedure. A triple-quadrupole MS/MS system operated in positive ion mode (source voltage, 5500 V) is used. Mass calibration and resolution of both resolving quadrupoles are optimized with a poly(propylene)glycol solution introduced by an infusion pump. Method optimization is performed by infusing a solution containing the measured enzyme products, LPC species and IS at 0.6 mL/h. The instrument is optimized to monitor the transitions. The single reaction monitoring experiments (100 ms dwell, each experiment) are added to the MS/MS method. Sample introduction into the atmospheric pressure ionization source is achieved by an autosampler and HPLC system. Autosampler injections of 20 μL per sample are made into the liquid chromatography (LC) mobile phase (800 mL methanol/200 mL water with 5 mmol/L ammonium formate) flow of 0.250 mL/min without the use of a chromatographic column. Between injections, analysis time is 1 min per sample. Each prepared sample with an apparently increased concentration of C₂₆-LPC was verified by a reflex analysis by use of LC-MS/MS as described by Turgeon (Turgeon, 2015) and Hubbard et al. (Hubbard, 2009) to minimize false-positive results. This is because an unidentified substance that is inconsistently present in DBS can interfere with accurate quantification of C₂₆-LPC by the flow injection tandem mass spectrometry (FIA-MS/MS) method).

Preparation of calibrators and controls. The skilled person would understand which controls are appropriate for the comparison of interest and would select these for use in the GCase activity protocol.

For example, controls for levels of enzyme activity can be prepared and supplied by the Centers for Disease Control and Prevention (CDC) in the USA. These controls include a base pool control (CDC Base Pool) deficient in enzyme activities, a low control (CDC low) with reduced enzyme activities, a medium control (CDC med) with moderate enzyme activities and a high control (CDC high) with normal enzyme activities. Liquid calibrators with product (P) to IS ratios (P/IS) of 0.00, 0.05, 0.5, 1.0, 2.0 and 5.0 for each enzyme can be supplied by the CDC, as well as liquid calibrators corresponding to 0.00, 0.58, 0.97, 1.16, 1.74 and 2.32 mg/L of C₂₀-, C₂₂-, C₂₄-, and C₂₆-LPC in a 3-mm DBS for use in linearity studies.

In some examples, controls for levels of enzyme activity may be prepared from samples taken from a patient before the start of treatment or from a patient at an earlier time point in treatment.

It may also be useful to provide controls prepared from a Base Pool deficient in enzyme activity, a Base Pool with low activity and/or a Base Pool of healthy subjects.

### Efficacy Results

Some subjects treated with Compound A exhibit an increased time to worsening compared to subjects treated with the placebo for at least one of the measures described herein. Clinical worsening can be defined as by 2-point increase in Movement Disorder Society-Unified Parkinson's Disease Rating Scale (MDS-UPDRS) Part II total score and no improvement in the Motor Examination, as assessed by 0-point increase in MDS-UPDRS Part III total score.

Alternatively or additionally, time to worsening can be defined as the time for a 5-point increase in the MDS-UPDRS Part III total score, or the time for a 3-point increase in the MDS-UPDRS Part III total score, and/or the time until a 2-point increase MDS-UPDRS Part II total score and ≥5-point increase on the Motor Examination assessed by the MDS-UPDRS Part III total score.

Alternatively or additionally, subjects treated with compound A may also exhibit an increased time from baseline to any worsening on the CGI-C, CGI-S, PGI-S, and/or PGI-C scales, and/or an increased time until First LEDD increase, compared to those subjects treated with placebo.

Alternatively or additionally, subjects treated with Compound A may exhibit a lower change from baseline to week 78 compared to those treated with placebo in MDS-UPDRS scores (I to IV), modified H&Y score, PD-CRS score, PDQ-39, EQ-5D-5L scores, CGI-C score, and/or PGI-C score.

### Pharmacokinetic Results

Mean plasma concentrations of Compound A are higher after the 60 mg dose than after the 10 mg dose at all measured time points.

### Pharmacodynamic Results

Mean change in plasma concentrations of neurofilament light chain are lower in subjects treated with Compound A compared to those treated with placebo.

Mean change in GCase activity in whole blood samples is higher in subjects treated with Compound A compared to those treated with placebo.

Mean change in lysosomal activity is higher in subjects treated with Compound A compared to those treated with placebo. Further, mean change in the level of GluSph is higher in subjects treated with Compound A compared to those treated with placebo.

Mean change of cerebral blood flow as measured by arterial spin labeling with MRI is higher in subjects treated with Compound A compared to those treated with placebo.

APPENDIX 1
List of GBA1 Mutations (for inclusion in trial)

| | Variant Name | | |
|---|---|---|---|
| Variant Name (Nucleotide)^{a, b} | (Protein)^{b} | Other Names^{c} | Severity^{d} |
| NM_001005741.3: c.1093G > A | p.Glu365Lys | E365K, E278K, E316K, E326K | Risk variant (mild) |
| NM_001005741.3: c.1223C > T | p.Thr408Met | T369M, T408M, T359M, T321M | Risk variant (mild) |
| NM_001005741.3: c.1226A > G | p.Asn409Ser | N409S, N322S, N360S, N370S | Mild |
| NM_001005741.3: c.1448T > C | p.Leu483Pro | L483P, L396P, L434P, L444P | Severe |
| NM_001005741.3: c.1604G > A | p.Arg535His | R535H, R486H, R448H, R496H | Mild |
| NM_001005741.3: c.1342G > C | p.Asp448His | D448H, D361H, D399H, D409H | Severe |
| NM_001005741.3: c.680A > G | p.Asn227Ser | N227S, N140S, N178S, N188S | Severe |
| NM_001005741.3: c.1504C > T | p.Arg502Cys | R502C, R415C, R453C, R463C | Severe |
| NM_001005741.3: c.84dup | p.Leu29Alafs*18 | L29fs, 84GG | Severe |
| NM_001005741.3: c.754T > A | p.Phe252Ile | F252I, F165I, F203I, F213I | Severe |
| NM_001005741.3: c.1297G > T | p.Val433Leu | V433L, V384L, V346L, V394L | Severe |
| NM_001005741.3: c.721G > A | p.Gly241Arg | G241R, G154R, G192R | Severe |
| NM_001005741.3: c.259C > T | p.Arg87Trp | R87W, 259C-T | Mild |
| NM_001005741.3: c.928A > G | p.Ser310Gly | S310G, S261G, S223G | Mild |
| NM_001005741.3: c.764T > A | p.Phe255Tyr | F255Y, F168Y, F206Y, F216Y | Mild |
| NM_001005741.3: c.1246G > A | p.Gly416Ser | G416S, G329S, G367S | Severe |
| NM_001005741.3: c.946C > T | p.Arg316Cys | R229C, R267C, R316C | Mild |
| NM_001005741.3: c.26_27del | p.Glu9Glyfs*8 | E9fs | Severe |
| NM_001005741.3: c.604C > T | p.Arg202* | R115, R153, R202 | Severe |
| NM_001005741.3: c.1090G > A | p.Gly364Arg | G364R, G277R, G315R, G325R | Severe |
| NM_001005741.3: c.1296G > A | p.Trp432* | W432, W345, W383 | Severe |
| NM_001005741.3: c.1192C > T | p.Arg398* | R398, R349, R311, R359 | Severe |
| NM_001005741.3: c.914del | p.Pro305Leufs*31 | P218fs, P256fs, P305fs | Severe |
| NM_001005741.3: c.256C > T | p.Arg86* | R86 | Severe |
| NM_001005741.3: c.586A > C | p.Lys196Gln | K196Q, K109Q, K147Q, K157Q | Severe |
| NM_001005741.3: c.1312G > A | p.Asp438Asn | D438N, D389N, D351N | Severe |
| NM_001005741.3: c.203dup | p.Thr69Aspfs*12 | T69fs | Severe |
| NM_001005741.3: c.475C > T | p.Arg159Trp | R159W, R110W, R72W | Severe |
| NM_001005741.3: c.476G > A | p.Arg159Gln | R159Q, R110Q, R72Q, R119Q | Severe |
| NM_001005741.3: c.887G > A | p.Arg296Gln | R296Q, R209Q, R247Q, R257E | Severe |
| NM_001005741.3: c.762-1G > C | p.? | | Severe |
| NM_001005741.3: c.115 + 1G > A | p.? | IVS2DS + 1G-A; IVS2DS, G-A, +1 | Severe |
| NM_001005741.3: c.1505 + 1G > T | p.? | | Severe |
| NM_001005741.3: c.123_217del | p.Pro42Trpfs*7, | P42Wfs*7, P42WfsTer7, | Severe |
| | p.Pro42TrpfsTer7 | P3Wfs*7, P3WfsTer7 | |
| NM_001005741.2: c.1265_1319del | p.Leu422ProfsTer4; | L422Pfs*4, L422PfsTer4, | Severe |
| | p.Leu422Profs*4; | L383Pfs*4, L383PfsTer4 | |
| NM_001005741.3: c.715C > T | p.Gln239*, | Q239*; Q239Ter, | Severe |
| | p.Gln239Ter | Q200*, Q200Ter | |
| NM_001005741.3: c.1085C > T | p.Thr362Ile | T362I, T323I | Severe |
| NM_001005741.3: c.413del | p.Pro138Leufs*62, | P138Lfs*62, P99Lfs*62 | Severe |
| | p.Pro138LeufsTer62 | | |
| NM_001005741.3: c.882T > G | p.His294Gln | H294Q, H255Q | Severe |
| NM_001005741.3: c.1193G > A | p.Arg398Gln | R398Q, R359Q | Mild |
| NM_001005741.3: c.1_2344del | p.Met1_*537del | M1_*537del, M1_Ter537del, | Severe |
| | | complete gene/alelle deletion | |

^{a}Subjects who have other GBA-PD risk-associated variants will be classified as either severe or mild based on emerging data and will be randomized accordingly.
^{b}Fulgent Genetics (Likely Pathogenic / Pathogenic).
^{c}National Library of Medicine.
^{d}See Parlar, 2023.

GBA-PD patients are enrolled when the following conditions are met:
a) heterozygous for any listed mutation, including heterozygous for “Risk variant (mild)” listed mutations, or
b) homozygous for “Risk variant (mild)” listed mutations.

GD patients (for exclusion) are:
1. homozygous for “Mild” or “Severe” listed mutations, or
2. compound heterozygous for “Mild” or “Severe” listed mutations.

APPENDIX 2
List of LRRK2 Mutations (for exclusion from trial)

| Variant Name (Nucleotide)^{a} | Variant Name (Protein)^{a} | Other Names^{b} |
|---|---|---|
| NM_198578.4: c.4309A > C | p.Asn1437His | N1437H |
| NM_198578.4: c.4321C > T | p.Arg1441Cys | R1441C |
| NM_198578.4: c.4322G > A | p.Arg1441His | R1441H |
| NM_198578.4: c.6055G > A | p.Gly2019Ser | G2019S |

^{a}Fulgent Genetics (Likely Pathogenic / Pathogenic).
^{b}National Library of Medicine.

### REFERENCES

The contents of the following documents and any documents cited elsewhere in this disclosure are incorporated herein by reference in their entirety.
ClinicalTrials.gov (2021). A Study to Evaluate the Efficacy of Prasinezumab (RO7046015/PRX002) in Participants With Early Parkinson's Disease (PASADENA). United States: ClinicalTrials.gov. Available: https://clinicaltrials.gov/ct2/show/NCT03318523
Den Heijer, J. M, Kruithof, A. C, Amerongen, G, de Kam, M. L, Thijssen E., Grievink, H. W, et al. A randomized single and multiple ascending dose study in healthy volunteers of LTI-291, a centrally penetrant glucocerebrosidase activator. British Journal of clinical Pharmacology, 2021; 87 (2); 3561-3571.
Gan-Or Z, Giladi N, Rozovski U, Shifrin C, Rosner S, Gurevich T, et al. Genotype-phenotype correlations between GBA mutations and Parkinson disease risk and onset. Neurology. 2008; 70(24):2277-83.
Gan-Or Z, Amshalom I, Kilarski L L, Bar-Shira A, Gana-Weisz M, Mirelman A, et al. Differential effects of severe vs mild GBA mutations on Parkinson disease. Neurology. 2015; 84(9):880-7.
Goetz C G, Poewe W, Rascol O, et al. Movement Disorder Society Task Force report on the Hoehn and Yahr staging scale: status and recommendations. Mov Disord. 2004; 19(9):1020-8.
Goetz C G, Tilley B C, Shaftman S R, et al. Movement Disorder Society-sponsored revision of the Unified Parkinson's Disease Rating Scale (MDS-UPDRS): scale presentation and clinimetric testing results. Mov Disord. 2008; 15; 23(15):2129-70.
Grabowski G A. Phenotype, diagnosis, and treatment of Gaucher's disease. Lancet. 2008; 372(9645):1263-71.
Guimarães Bde C, Pereira A C, Rodrigues Fda C, Vaz dos Santos A, Campos Jr M, Mendonca dos Santos J, et al. Glucocerebrosidase N370S and L444P mutations as risk factors for Parkinson's disease in Brazilian patients. Parkinsonism Relat Disord. 2012; 18(5):688-9.
Guy W, editor. ECDEU Assessment Manual for Psychopharmacology. Rockville, MD: US Department of Health, Education, and Welfare Public Health Service Alcohol, Drug Abuse, and Mental Health Administration; 1976.
Herdman M, Gudex C, Lloyd A, et al. Development and preliminary testing of the new five-level version of EQ-5D (EQ-5D-5L). Qual Life Res. 2011; 20(10):1727-36.
Hubbard W C, Moser A B, Liu A C, Jones R O, Steinberg S J, Lorey F, et al. Newborn screening for X-linked adrenoleukodystrophy (X-ALD): validation of a combined liquid chromatography-tandem mass spectrometric (LC-MS/MS) method. Mol Genet Metab 2009; 97: 212-20.
Jesús S, Huertas I, Bernal-Bernal I, Bonilla-Toribio M, Caceres-Redondo M T, Vargas-Gonzalez L, et al. GBA variants influence motor and non-motor features of Parkinson's disease. PLoS One. 2016; 11(12):e0167749.
Leyns C, et al. Glucocerebrosidase activity and lipid levels are related to protein pathologies in Parkinson's disease. NPJ Parkinsons Dis. 2023 May 11; 9(1):74.
Liu G, Boot B, Locascio J J, Jansen I E, Winder-Rhodes S, Eberly S, et al. Specifically neuropathic Gaucher's mutations accelerate cognitive decline in Parkinson's. Ann Neurol. 2016; 80(5):674-85.
Mata I F, Leverenz J B, Weintraub D, Trojanowski J Q, Chen-Plotkin A, Van Deerlin V M, et al. GBA Variants are associated with a distinct pattern of cognitive deficits in Parkinson's disease. Mov Disord. 2016; 31(1):95-102.
Moraitou M, Hadjigeorgiou G, Monopolis I, Dardiotis E, Bozi M, Vassilatis D, et al. β-Glucocerebrosidase gene mutations in two cohorts of Greek patients with sporadic Parkinson's disease. Mol Genet Metab. 2011; 104(1 2):149-52.
Neudorfer O, Giladi N, Elstein D, Abrahamov A, Turezkite T, Aghai E, et al. Occurrence of Parkinson's syndrome in type 1 Gaucher disease. QJM. 1996; 89(9):691-4.
Neumann J, Bras J, Deas E, O'Sullivan S S, Parkkinen L, Lachmann R H, et al. Glucocerebrosidase mutations in clinical and pathologically proven Parkinson's disease. Brain. 2009; 132(Pt 7):1783-94.
Pagonabarraga J, Kulisevsky J, Llebaria G, et al. Parkinson's disease-cognitive rating scale: A new cognitive scale specific for Parkinson's disease. Mov Disord. 2008; 23(7):998-1005.
Parlar S C, Grenn F P, Kim J J, et al. Classification of GBA1 variants in Parkinson's disease: the GBA1-PD Browser. Mov Disord. 2023 Jan. 4. doi: 10.1002/mds.29314.
Peterschmitt M. J., Giladi N., Alcalay R. N., Simuni T., Marek K., Investigators M.-P. (2021a). “Ven-glustat In Parkinson's Disease Patients with a GBA Mutation: results From Part 2 of the Phase 2 MOVES-PD Trial” in 15th International Conference on Alzheimer's & Parkinson's Diseases (Virtual;
Peto V, Jenkinson C, Fitzpatrick R. PDQ-39: a review of the development, validation and application of a Parkinson's disease quality of life questionnaire and its associated measures. J Neurol. 1998; 245(Suppl 1):S10-4.
Pires N et al. (2023) Profile Of Glucocerebrosidase 1 (GCase) Activity And Glucosylsphingosine Levels In GBA-PD, PD Patients And Healthy Volunteers. International Conference on Alzheimer's and Parkinson's Diseases and related neurological disorders. AD/PD 2024 Posters. P1124/#1065.
Posner K, Brown G K, Stanley B, et al. The Columbia-Suicide Severity Rating Scale: Initial validity and internal consistency findings from three multisite studies with adolescents and adults. Am J Psych. 2011; 168(12):1266-77.
Tilley B. C., Galpern W. R. (2007). Screening potential therapies: lessons learned from new paradigms used in Parkinson disease. Stroke 38 800-803.
Rascol O., Hauser R. A., Stocchi F., Fitzer-Attas C. J., Sidi Y., Abler V., et al. (2016). Long-term effects of rasagiline and the natural history of treated Parkinson's disease. Mov. Disord. 31 1489-1496.
Rosenbloom B, Balwani M, Bronstein J M, Kolodny E, Sathe S, Gwosdow A R, et al. The incidence of Parkinsonism in patients with type 1 Gaucher disease: data from the ICGG Gaucher Registry. Blood Cells Mol Dis. 2011; 46(1):95-102.
Rosenbloom B E, Weinreb N J. Gaucher disease: a comprehensive review. Crit Rev Oncog. 2013; 18(3):163-75.
Surface M et al. (2022) Plasma Glucosylsphingosine in GBA1 Mutation Carriers with and without Parkinson's Disease. Mov Disord, 37: 416-421.
Tortorelli S, Turgeon C T, Gavrilov D K, Oglesbee D, Raymond K M, Rinaldo P, Matern D. Simultaneous Testing for 6 Lysosomal Storage Disorders and X-Adrenoleukodystrophy in Dried Blood Spots by Tandem Mass Spectrometry. Clin Chem. 2016 September; 62(9):1248-54. doi: 10.1373/clinchem.2016.256255. Epub 2016 Jul. 20. PMID: 27440509.
Turgeon C T, Moser A B, Morkrid L, Magera M J, Gavrilov D K, Oglesbee D, et al. Streamlined determination of lysophosphatidylcholines in dried blood spots for new-born screening of X-linked adrenoleukodystrophy. Mol Genet Metab 2015; 114:46-50.
US Food and Drug Administration (FDA). Guidance for Industry: E10 choice of control group and related issues in clinical trials; May 2001.
US Food and Drug Administration (FDA). Guidance for Industry: Multiple endpoints in clinical trials; January 2017.
Ysselstein D, Young T J, Nguyen M, Padmanabhan S, Hirst W D, Dzamko N, Krainc D, Evaluation of Strategies for Measuring Lysosomal Glucocerebrosidase Activity, Movement Disorders, 2021; 36(12):2719-2730

Abbreviations

| Abbreviation | Definition |
|---|---|
| 4-MUG | 4-methylumbelliferyl-beta-D-glucopyranoside |
| 5-HT_{1A} | Serotonin 1A receptor |
| 5-HT₆ | Serotonin 6 receptor |
| ADME | Absorption, distribution, metabolism, and elimination |
| AE | Adverse event |
| A/G | Albumin and globulin ratio |
| ALD | X-adrenoleukodystrophy |
| ALT | Alanine aminotransferase |
| ANCOVA | Analysis of covariance |
| ASM | Acid sphingomyelinase |
| AST | Aspartate aminotransferase |
| AUC | Area under the concentration-time curve |
| AUC₀₋ₜ or | Area under the concentration-time curve from time |
| AUC₀₋ₗₐₛₜ | zero to the last quantifiable concentration |
| AUC₀₋₂₄ | Area under the concentration-time curve from time |
| | zero to 24 hours postdose |
| AUC_{0-inf} | Area under the concentration-time curve from time |
| | zero extrapolated to infinity |
| BCRP | Breast cancer resistance protein |
| Caco-2 | Colorectal adenocarcinoma |
| CBE | Conduritol B epoxide |
| CDC | Centers for Disease Control and Prevention |
| cDNA | Complementary DNA |
| CL | Clearance |
| CL/F | Apparent oral clearance, calculated as dose/AUC_{0-inf} |
| Cₘₐₓ | Maximum concentration |
| Cₘᵢₙ | Minimum concentration |
| CNS | Central nervous system |
| CSF | Cerebrospinal fluid |
| C-SSRS | Columbia-Suicide Severity Rating Scale |
| CYP | Cytochrome P450 |
| DDI | Drug drug interaction |
| DBS | Dried blood spot |
| DNA | Deoxyribonucleic acid |
| ECG | Electrocardiogram |
| EEG | Pharmaco-electroencephalogram |
| ER | Extended release |
| FGD-PET | Fluorodeoxyglucose-positron emission tomography |
| FIH | First-in-human |
| FIA-MS/MS | Flow injection tandem mass spectrometry |
| fMRI | Functional magnetic resonance imaging |
| GAA | Acid α-glucosidase |
| GBA1 | Parkinson's disease due to a pathogenic variant in the |
| | GBA1 gene |
| GBA-PD | Parkinson's disease due to a pathogenic variant in the |
| | GBA1 gene |
| GCase | Beta-glucocerebrosidase |
| GD | Gaucher disease |
| GGT | Gamma-glutamyl transferase |
| GI | Gastrointestinal |
| GLA | α-galactosidase A |
| GLP | Good Laboratory Practice |
| GluCer | Glucosylceramide |
| GluSph | Glucosylsphingosine |
| GSL | Glycosphingolipid |
| HEK293 | Human embryonic kidney 293 |
| hERG | Human ether-à-go-go-related gene |
| HPLC | High-performance liquid chromatography |
| IC₅₀ | 50% maximal inhibition |
| IMP | Investigational medicinal product |
| iPD | Idiopathic Parkinson's disease |
| iPSC | Induced pluripotent stem cell |
| IR | Immediate release |
| IS | Internal Standard |
| IDUA | α-L-iduronidase |
| IV | Intravenous |
| LacCer | Lactosylceramide |
| LC | Liquid chromatography |
| LC-MS/MS | Liquid chromatography quadrupole tandem mass |
| | spectrometry. |
| LDH | Lactate dehydrogenase |
| LPC | Lysophosphatidylcholines |
| LSD | Lysosomal storage disorders |
| MATE1 | Multidrug and toxic extrusion protein 1 |
| MCI | Mild Cognitive Impairment |
| MDR1 | Multidrug resistance protein 1 |
| MDS-UPDRS | Movement Disorder Society-Unified Parkinson's |
| | Disease Rating Scale |
| MMSE | Min-Mental State Examination |
| mRNA | Messenger ribonucleic acid |
| MTD | Maximum tolerated dose |
| NOAEL | No observed adverse effect level |
| OAT1, | Organic anion transporter 1 |
| OAT3 | Organic anion transporter 3 |
| OATP1B1 | Organic anion-transporting polypeptide 1B1 |
| OATP1B3 | Organic anion-transporting polypeptide 1B3 |
| OCT2 | Organic cation transporter 2 |
| P | Product |
| Pₐₚₚ | Apparent permeability |
| PBMC | Peripheral blood mononuclear cells |
| PD | Parkinson's disease |
| P-gp | Permeability-glycoprotein |
| PK | Pharmacokinetic(s) |
| PS | Phosphatidylserine |
| QTc | QT interval corrected for heart rate |
| QTcF | QT interval corrected for heart rate based on the |
| | Fridericia correction |
| RBC | Red blood cell |
| S | Substrate |
| SAE | Serious adverse event |
| SS | Steady state |
| SUSAR | Suspected unexpected serious adverse reaction |
| SynTg | Transgenic mice over-expressing human wild type |
| | alpha-synuclein under the control of the human |
| | Thy1 promoter [TNWT61 or also Line 61] |
| t_{1/2} | Terminal half-life |
| TEAE | Treatment-emergent adverse event |
| Tₘₐₓ or tₘₐₓ | Time of maximum observed concentration |
| Vₘₐₓ | Maximal velocity |
| Vₛₛ | Volume of distribution at steady state |
| Vz/F | Apparent volume of distribution at terminal phase |
| VVLT | Visual Verbal Learning Test |
| WBC | White blood cell |

1. 5,7-dimethyl-N-((1R,4R)-4-(pentyloxy)cyclohexyl)pyrazolol[1,5-a]pyrimidine-3-carboxamide (Compound A), or a pharmaceutically acceptable salt thereof, for use in preventing or limiting clinical motor progression in a subject with Parkinson's disease who has a pathogenic variant in the glucocerebrosidase 1 (GBA1) gene (GBA-PD).

2. A method for preventing or limiting clinical motor progression in a subject with Parkinson's disease who has a pathogenic variant in the glucocerebrosidase 1 (GBA1) gene (GBA-PD), said method comprising administering a therapeutically effective amount of 5,7-dimethyl-N-((1R,4R)-4-(pentyloxy)cyclohexyl)pyrazolol[1,5-a]pyrimidine-3-carboxamide (Compound A), or a pharmaceutically acceptable salt thereof, to said subject.

3. 5,7-dimethyl-N-((1R,4R)-4-(pentyloxy)cyclohexyl)pyrazolol[1,5-a]pyrimidine-3-carboxamide (Compound A), or a pharmaceutically acceptable salt thereof, for use in the treatment or prevention of Parkinson's Disease in a subject who has a pathogenic variant in the glucocerebrosidase 1 (GBA1) gene (GBA-PD) and who does not have a PD associated pathogenic variant in LRRK2.

4. A method for treating or preventing Parkinson's Disease in a subject with a pathogenic variant in the glucocerebrosidase 1 (GBA1) gene (GBA-PD) and who does not have a PD associated pathogenic variant in LRRK2, said method comprising administering a therapeutically effective amount of 5,7-dimethyl-N-((1R,4R)-4-(pentyloxy)cyclohexyl)pyrazolol[1,5-a]pyrimidine-3-carboxamide (Compound A), or a pharmaceutically acceptable salt thereof, to said subject.

5. 5,7-dimethyl-N-((1R,4R)-4-(pentyloxy)cyclohexyl)pyrazolol[1,5-a]pyrimidine-3-carboxamide (Compound A), or a pharmaceutically acceptable salt thereof, for use in the treatment or prevention of Parkinson's Disease in a subject who has a pathogenic variant in the glucocerebrosidase 1 (GBA1) gene (GBA-PD) and who does not suffer from Gaucher's disease.

6. A method for treating or preventing Parkinson's Disease in a subject who has a pathogenic variant in the glucocerebrosidase 1 (GBA1) gene (GBA-PD) and who does not suffer from Gaucher's Disease, said method comprising administering a therapeutically effective amount of 5,7-dimethyl-N-((1R,4R)-4-(pentyloxy)cyclohexyl)pyrazolol[1,5-a]pyrimidine-3-carboxamide (Compound A), or a pharmaceutically acceptable salt thereof, to said subject.

7. 5,7-dimethyl-N-((1R,4R)-4-(pentyloxy)cyclohexyl)pyrazolol[1,5-a]pyrimidine-3-carboxamide (Compound A), or a pharmaceutically acceptable salt thereof, for use in the treatment or prevention of Parkinson's Disease in a subject who has a pathogenic variant in the glucocerebrosidase 1 (GBA1) gene (GBA-PD) and who is not homozygous for a GBA1 pathogenic variant that is known to be associated with Gaucher's Disease or compound heterozygous for two alleles of GBA1 that are known to be associated with Gaucher's Disease.

8. A method for treating or preventing Parkinson's Disease in a subject with a pathogenic variant in the glucocerebrosidase 1 (GBA1) gene (GBA-PD) and who is not homozygous for a GBA1 pathogenic variant that is known to be associated with Gaucher's Disease or compound heterozygous for two alleles of GBA1 that are known to be associated with Gaucher's Disease, said method comprising administering a therapeutically effective amount of 5,7-dimethyl-N-((1R,4R)-4-(pentyloxy)cyclohexyl)pyrazolol[1,5-a]pyrimidine-3-carboxamide (Compound A), or a pharmaceutically acceptable salt thereof, to said subject.

9. A method of preventing or delaying cognitive impairment in a subject with Parkinson's disease who has a pathogenic variant in the glucocerebrosidase 1 (GBA1) gene (GBA-PD), said method comprising administering a therapeutically effective amount of 5,7-dimethyl-N-((1R,4R)-4-(pentyloxy)cyclohexyl)pyrazolol[1,5-a]pyrimidine-3-carboxamide (Compound A), or a pharmaceutically acceptable salt thereof, to said subject.

10. 5,7-dimethyl-N-((1R,4R)-4-(pentyloxy)cyclohexyl)pyrazolol[1,5-a]pyrimidine-3-carboxamide (Compound A), or a pharmaceutically acceptable salt thereof, for use in preventing or delaying cognitive impairment in a subject with Parkinson's disease who has a pathogenic variant in the glucocerebrosidase 1 (GBA1) gene (GBA-PD).

11. A method of treating Parkinson's disease in a subject having decreased, reduced or low GCase activity, said method comprising administering a therapeutically effective amount of Compound A, or a pharmaceutically acceptable salt thereof, to said subject.

12. 5,7-dimethyl-N-((1R,4R)-4-(pentyloxy)cyclohexyl)pyrazolol[1,5-a]pyrimidine-3-carboxamide (Compound A), or a pharmaceutically acceptable salt thereof, for use in the treatment of Parkinson's disease in a subject having decreased, reduced, or low GCase activity.

13. The method of treatment or use according to any one of the preceding claims, wherein treatment or clinical motor progression is assessed using the Movement Disorder Society-Unified Parkinson's Disease Rating Scale (MDS-UPDRS), the Clinical Global Impression-Change (CGI-C) scale, and/or the Patient Global Impression-Change (PGI-C) scale.

14. The method of treatment or use according to any one of the preceding claims, wherein said use or treatment results in increased time for a ≥2-point increase in the MDS-UPDRS Part II score and no improvement (i.e., score of zero or higher) in the Part III score compared to a subject treated with a placebo.

15. The method of treatment or use according to any one of the preceding claims, wherein said use or treatment results in increased time for a ≥2-point increase in the MDS-UPDRS Part II score and no improvement (i.e., difference from baseline of zero or higher) in the Part III score compared to a subject treated with a placebo.

16. The method of treatment or use according to any one of the preceding claims, wherein said use or treatment results in increased time for a ≥5-point increase in the MDS-UPDRS Part III total score compared to a subject treated with a placebo.

17. The method of treatment or use according to any one of the preceding claims, wherein said use or treatment results in increased time for a ≥3-point increase in the MDS-UPDRS Part III total score compared to a subject treated with a placebo.

18. The method of treatment or use according to any one of the preceding claims, wherein said use or treatment results in increased time for a ≥2-point increase in the MDS-UPDRS Part II score and confirmed by ≥5-point increase in the MDS-UPDRS Part III score compared to a subject treated with a placebo.

19. The method of treatment or use according to any one of the preceding claims, wherein said use or treatment results in increased time compared to a subject treated with a placebo until:
i) first levodopa-equivalent daily dosage increase (LEDD);
ii) any worsening on the Clinical Global Impression-Corrections (CGI-C) scale;
iii) any worsening on the Patient Global Impression-Corrections (PGI-C) scale;
iv) any worsening on the Clinical Global Impression-Severity (CGI-S) scale; or
v) any worsening on the Patient Global Impression-Corrections (PGI-S) scale.

20. The method of treatment or use according to any one of the preceding claims, wherein said use or treatment results in an improved gait speed or said use or treatment prevents, limits or delays a decline in gait speed.

21. The method of treatment or use according to any one of the preceding claims, wherein said use or treatment results in improved cerebral blood flow as measured using arterial spin labelling and/or MRI free-water imaging.

22. The method of treatment or use according to any one of the preceding claims, wherein said use or treatment results in reduced neurofilament light chain concentrations.

23. The method of treatment or use according to any one of the preceding claims, wherein said use or treatment results in preventing or limiting quality of life deterioration.

24. The method of treatment or use according to any one of the preceding claims, wherein said use or treatment results in an improvement in quality of life.

25. The method of treatment or use according to any one of the preceding claims, whereby the GCase activity in the cerebrospinal fluid (CSF) of the subject at least doubles.

26. The method of treatment or use according to any one of the preceding claims, wherein the subject has been clinically diagnosed as having Parkinson's disease, for example GBA-PD.

27. The method of treatment or use according to any one of the preceding claims, wherein the subject has a clinical diagnosis of PD for at least 1 year and for no longer than 7 years before initiation of treatment, the subject has a modified Hoehn and Yahr score 2.5 and the subject is receiving symptomatic treatment for PD.

28. The method of treatment or use according to any one of the preceding claims, wherein said subject has been diagnosed as having Parkinson's disease and having decreased, reduced, or low GCase activity.

29. The method of treatment or use according to any one of the preceding claims, wherein Compound A is administered in combination with a therapeutically effective amount of one or more of the following concomitant drugs: Dopaminergic agents, Dopamine receptor agonists, Monoamine oxidase B inhibitors, Catechol-O-methyltransferase inhibitors, N methyl-D-Aspartate Receptor antagonists, Adenosine receptor antagonists, and Anticholinergic agents.

30. The method of treatment or use according to any one of the preceding claims, wherein Compound A, or a pharmaceutically acceptable salt thereof, is administered in combination with a therapeutically effective amount of a Dopaminergic agent.

31. The method of treatment or use according to claim 30, wherein Compound A, or a pharmaceutically acceptable salt thereof, is administered in combination with a therapeutically effective amount of levodopa or a levodopa/DDCI preparation.

32. The method of treatment or use according to claim 31, wherein Compound A, or a pharmaceutically acceptable salt thereof, and levodopa or a levodopa/DDCI preparation are administered simultaneously or sequentially within less than 30 minutes.

33. The method of treatment or use according to claim 32, wherein Compound A, or a pharmaceutically acceptable salt thereof, and levodopa or a levodopa/DDCI preparation are administered separately, at least 30 minutes apart.

34. 5,7-dimethyl-N-((1R,4R)-4-(pentyloxy)cyclohexyl)pyrazolol[1,5-a]pyrimidine-3-carboxamide (Compound A), or a pharmaceutically acceptable salt thereof, for use in preventing or reducing the risk of Parkinson's disease in a subject determined as being at risk of Parkinson's disease, for example, a subject not diagnosed as having Parkinson's disease and determined as carrying a GBA1 pathogenic variant for PD.

35. A method for preventing or reducing the risk of Parkinson's disease in a subject determined as being at risk of Parkinson's disease, for example, a subject not diagnosed as having Parkinson's disease and determined as carrying a GBA1 pathogenic variant for PD, said method comprising administering a therapeutically effective amount of Compound A, or a pharmaceutically acceptable salt thereof, to said subject.

36. The method of treatment or use according to claim 34 or 35, wherein said subject has been determined as having decreased, reduced, or low GCase activity.

37. The method of treatment or use according to any one of claims 34 to 36, whereby the GCase activity in the cerebrospinal fluid (CSF) of the subject at least doubles.

38. The method of treatment or use according to any one of claims 34 to 37, wherein the subject has been determined as carrying a GBA1 pathogenic variant for PD.

39. The method of treatment or use according to any one of claims 34 to 38, wherein said use or treatment results in preventing or delaying the onset of Parkinson's disease.

40. The method of treatment or use according to any one of claims 34 to 39, wherein said use or treatment results in preventing, limiting or delaying quality of life deterioration.

41. The method of treatment or use according to any one of the preceding claims, wherein said use or treatment comprises administering to the subject a dose of about 10 mg to about 60 mg Compound A per day.

42. The method of treatment or use according to any one of the preceding claims, wherein said use or treatment comprises administering to the subject a dose of about 10 mg of Compound A per day.

43. The method of treatment or use according to any one of the preceding claims, wherein said use or treatment comprises administering to the subject a dose of about 60 mg of Compound A per day.

44. The method of treatment or use according to any one of the preceding claims, wherein Compound A, or a pharmaceutically acceptable salt thereof, is used or administered to a subject for a prolonged period of time, for example, for 52 weeks or more.

45. The method of treatment or use according to any one of the preceding claims, wherein Compound A, or a pharmaceutically acceptable salt thereof, is used or administered to a subject once-daily.

46. The method of treatment or use according to any one of claims 1-10 and 13-45, wherein the pathogenic variant in the GBA1 gene is selected from one of the following nucleotide variations: NM_001005741.3:c.1093G>A, NM_001005741.3:c.1223C>T, NM_001005741.3:c.1226A>G, NM_001005741.3:c.1448T>C, NM_001005741.3:c.1604G>A, NM_001005741.3:c.1342G>C, NM_001005741.3:c.680A>G, NM_001005741.3:c.1504C>T, NM_001005741.3:c.84dup, NM_001005741.3: c.754T>A, NM_001005741.3: c.1297G>T, NM_001005741.3: c.721G>A, NM_001005741.3: c.259C>T, NM_001005741.3: c.928A>G, NM_001005741.3: c.764T>A, NM_001005741.3:c.1246G>A, NM_001005741.3:c.946C>T, NM_001005741.3:c.26_27del, NM_001005741.3:c.604C>T, NM_001005741.3:c.1090G>A, NM_001005741.3:c.1296G>A, NM_001005741.3: c.1192C>T, NM_001005741.3: c.914del, NM_001005741.3:c.256C>T, NM_001005741.3:c.586A>C, NM_001005741.3:c.1312G>A, NM_001005741.3:c.203dup, NM_001005741.3: c.475C>T, NM_001005741.3: c.476G>A, NM_001005741.3:c.887G>A, NM_001005741.3:c.762-1G>C, NM_001005741.3:c.115+1G>A, NM_001005741.3:c.1505+1G>T, NM_001005741.3:c.123_217del, NM_001005741.2:c.1265_1319del, NM_001005741.3:c.715C>T, NM_001005741.3:c.1085C>T, NM_001005741.3:c.413del, NM_001005741.3:c.882T>G, NM_001005741.3:c.1193G>A and NM_001005741.3:c.1_2344del.
